(19) United States
(12) Patent Application Publication
(10) Pub. No.: US 2024/0241444 A1
(43) Pub. Date: Jul. 18, 2024

(54) ACTINIC RAY-SENSITIVE OR RADIATION-SENSITIVE RESIN COMPOSITION, ACTINIC RAY-SENSITIVE OR RADIATION-SENSITIVE FILM, PATTERN FORMING METHOD, AND METHOD FOR MANUFACTURING ELECTRONIC DEVICE

(71) Applicant: FUJIFILM Corporation, Tokyo (JP)

(72) Inventor: Shuhei YAMAGUCHI, Shizuoka (JP)

(73) Assignee: FUJIFILM Corporation, Tokyo (JP)

(21) Appl. No.: 18/426,343

(22) Filed: Jan. 30, 2024

Related U.S. Application Data
(63) Continuation of application No. PCT/JP2022/027571, filed on Jul. 13, 2022.

(30) Foreign Application Priority Data
Aug. 12, 2021 (JP) 2021-131755

(51) Int. Cl. G03F 7/039; G03F 7/004

(52) U.S. Cl. CPC G03F 7/0392; G03F 7/0045

(57) ABSTRACT

An actinic ray-sensitive or radiation-sensitive resin composition includes: a resin (A) that has a specific repeating unit and whose polarity increases by the action of an acid; and an acidic compound (F) having an iodine atom. The resin (A) is a different compound from the acidic compound (F), and the acidic compound (F) is a nonionic compound.

### CROSS REFERENCE TO RELATED APPLICATION

This is a continuation of International Application No. PCT/JP2022/027571 filed on Jul. 13, 2022, and claims priority from Japanese Patent Application No. 2021-131755 filed on Aug. 12, 2021, the entire disclosures of which are incorporated herein by reference.

### BACKGROUND OF THE INVENTION

### 1. Field of the Invention

The present invention relates to an actinic ray-sensitive or radiation-sensitive resin composition, an actinic ray-sensitive or radiation-sensitive film, a pattern forming method, and a method for manufacturing an electronic device. More particularly, the invention relates to an actinic ray-sensitive or radiation-sensitive resin composition that is preferably used for ultramicrolithography processes applicable to processes for manufacturing VLSI (very large scale integration) circuits and high-capacity microchips, to processes for producing molds for nanoimprinting, to processes for manufacturing high-density information recording mediums, and to other processes and also applicable to other photofabrication processes and also relates to an actinic ray-sensitive or radiation-sensitive film, a pattern forming method, and a method for manufacturing an electronic device.

### 2. Description of the Related Art

Conventional manufacturing processes for semiconductor devices such as ICs (Integrated Circuits) or LSI circuits involve lithographic microfabrication using a photoresist composition. In recent years, the degree of integration of integrated circuits has increased, and there is a growing demand for ultra-fine pattern formation in the submicron or quarter-micron range. Accordingly, the wavelength of exposure light tends to be shortened. Specifically, the g-line is replaced by the i-line and then by KrF excimer laser light. At present, exposure devices using an ArF excimer laser with a wavelength of 193 nm as the light source are being developed. In addition, techniques for further increasing resolving power are under development. Specifically, the so-called immersion method is under development, in which the space between a projection lens and a specimen is filled with a high-refractive index liquid (hereinafter referred to as “immersion liquid”).

At present, in addition to the use of excimer laser light, lithography using electron beams (EB), X-rays, extreme ultraviolet rays (EUV), etc. is being developed. This leads to the development of chemical amplification resist compositions that are effectively sensitive to various types of radiation and exhibit high sensitivity and resolution.

For example, WO2018/180049A and JP2021-67934A disclose resist compositions each including a compound having an iodine atom.

### SUMMARY OF THE INVENTION

It is stated in WO2018/180049A and JP2021-67934A that the use of the compound having an iodine atom can improve the sensitivity of the resist composition.

However, in recent years, the demands on the performance of resist compositions have continued to increase due to the reduction in the size of patterns to be formed etc. In particular, there is a need to further improve resolution and bridge margin.

It is an object of the invention to provide an actinic ray-sensitive or radiation-sensitive resin composition capable of providing good resolution and good bridge margin. It is another object of the invention to provide an actinic ray-sensitive or radiation-sensitive film using the actinic ray-sensitive or radiation-sensitive resin composition, a pattern forming method, and a method for manufacturing an electronic device.

The inventors have found that the above objects can be achieved by the following aspects.

[1] An actinic ray-sensitive or radiation-sensitive resin composition including: a resin (A) whose polarity increases by the action of an acid; and an acidic compound (F) having an iodine atom,
wherein the resin (A) is a different compound from the acidic compound (F),
wherein the acidic compound (F) is a nonionic compound, and
wherein the resin (A) has at least one selected from the group consisting of a repeating unit represented by general formula (3) below, a repeating unit represented by general formula (6) below, and a repeating unit represented by general formula (7) below:

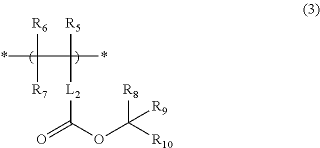

In general formula (3),

R₅ to R₇ each independently represent a hydrogen atom, an alkyl group, a cycloalkyl group, a halogen atom, a cyano group, or an alkoxycarbonyl group.

L₂ represents a divalent linking group.

R₈ to R₁₀ each independently represent an alkyl group, a cycloalkyl group, an aryl group, an aralkyl group, or an alkenyl group. Two selected from the group consisting of R₈ to R₁₀ may be bonded together to form a ring.

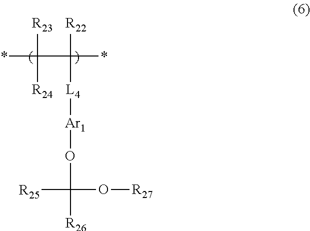

In general formula (6),

R₂₂ to R₂₄ each independently represent a hydrogen atom, an alkyl group, a cycloalkyl group, a halogen atom, a cyano group, or an alkoxycarbonyl group.

L₄ represents a single bond or a divalent linking group.

Ar₁ represents an aromatic group.

R₂₅ to R₂₇ each independently represent a hydrogen atom, an alkyl group, a cycloalkyl group, an aryl group, an aralkyl group, or an alkenyl group.

R₂₆ and R₂₇ may be bonded together to form a ring.

R₂₄ or R₂₅ may be bonded to Ar₁.

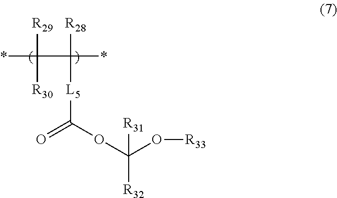

In general formula (7),

R₂₈ to R₃₀ each independently represent a hydrogen atom, an alkyl group, a cycloalkyl group, a halogen atom, a cyano group, or an alkoxycarbonyl group.

L₅ represents a single bond or a divalent linking group.

R₃₁ and R₃₂ each independently represent a hydrogen atom, an alkyl group, a cycloalkyl group, an aryl group, an aralkyl group, or an alkenyl group.

R₃₃ represents an alkyl group, a cycloalkyl group, an aryl group, an aralkyl group, or an alkenyl group.

R₃₂ and R₃₃ may be bonded together to form a ring.

[2] The actinic ray-sensitive or radiation-sensitive resin composition according to [1], wherein the acidic compound (F) is a compound having an aromatic group substituted with an iodine atom.

[3] The actinic ray-sensitive or radiation-sensitive resin composition according to [1] or [2], wherein the acidic compound (F) is a compound represented by the following general formula (FA1):

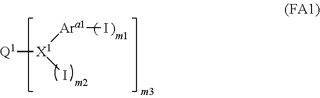

In general formula (FA1),

Ar^{a1} represents an aromatic group.

X¹ represents a single bond or a linking group.

Q¹ represents an acidic group.

Q¹ and Ar^{a1} may be bonded together to form a ring.

m1 and m2 each independently represent an integer of 0 to 5, provided that m1+m2 is 1 to 6.

When X¹ represents a single bond, m2 represents 0.

m3 represents 1 or 2.

When m3 is 2, two Ar^{a1}'s may be the same or different, and two X's may be the same or different.

[4] The actinic ray-sensitive or radiation-sensitive resin composition according to [1] or [2], wherein the acidic compound (F) is a compound represented by the following general formula (FA2):

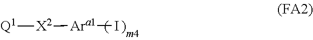

In general formula (FA2),

Arₐ₁ represents an aromatic group.

X² represents a single bond or a divalent linking group.

Q¹ represents an acidic group.

m4 represents an integer of 1 to 5.

[5] The actinic ray-sensitive or radiation-sensitive resin composition according to [1] or [2], wherein the acidic compound (F) is a compound represented by the following general formula (FA3):

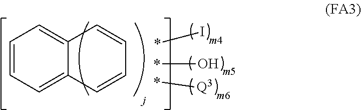

In general formula (FA3).

j represents 0 or 1.

Q³ represents a substituent.

m4 represents an integer of 1 to 5.

m5 represents an integer of 1 or more and (6+2j−m4) or less.

m6 represents an integer of 0 or more and (6+2j−m4−m5) or less.

Each * represents a direct bond bonded to an aromatic hydrocarbon shown in general formula (FA3).

[6] The actinic ray-sensitive or radiation-sensitive resin composition according to [1] or [2], wherein the acidic compound (F) is a compound represented by the following general formula (FA4):

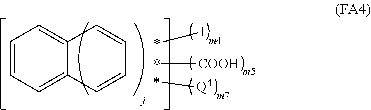

In general formula (FA4),

j represents 0 or 1.

Q⁴ represents a substituent.

m4 represents an integer of 1 to 5.

m5 represents an integer of 1 or more and (6+2j−m4) or less.

m7 represents an integer of 0 or more and (6+2j−m4−m5) or less.

Each * represents a direct bond bonded to an aromatic hydrocarbon shown in general formula (FA4).

[7] The actinic ray-sensitive or radiation-sensitive resin composition according to [1] or [2], wherein the acidic compound (F) is a compound represented by the following general formula (FA5):

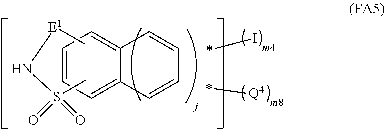

In general formula (FA5),

j represents 0 or 1.

Q⁴ represents a substituent.

E¹ represents a single bond or a divalent linking group.

m4 represents an integer of 1 to 5.

m8 represents an integer of 0 or more and (4+2j−m4) or less.

Each * represents a direct bond bonded to an aromatic hydrocarbon shown in general formula (FA5).

[8] The actinic ray-sensitive or radiation-sensitive resin composition according to any one of [1] to [7], wherein the acidic compound (F) has a pKa of 6 or less.

[9] The actinic ray-sensitive or radiation-sensitive resin composition according to any one of [1] to [8], wherein the acidic compound (F) has a molecular weight of 1000 or less.

[10] The actinic ray-sensitive or radiation-sensitive resin composition according to any one of [1] to [9], wherein the resin (A) has a repeating unit represented by the following general formula (A2):

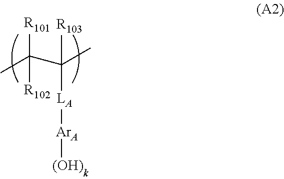

In general formula (A2),

R₁₀₁, R₁₀₂, and R₁₀₃ each independently represent a hydrogen atom, an alkyl group, a cycloalkyl group, a halogen atom, a cyano group, or an alkoxycarbonyl group.

L_{A} represents a single bond or a divalent linking group.

Ar_{A} represents an aromatic group.

k represents an integer of 1 to 5.

R₁₀₂ and Ar_{A} may be bonded together. When R₁₀₂ and Ar_{A} are bonded together, R₁₀₂ represents a single bond or an alkylene group.

[11] The actinic ray-sensitive or radiation-sensitive resin composition according to any one of [1] to [10], further including a compound that generates an acid upon irradiation with actinic rays or radiation.

[12] An actinic ray-sensitive or radiation-sensitive film formed from the actinic ray-sensitive or radiation-sensitive resin composition according to any one of [1] to [11].

[13] A pattern forming method including the steps of:

forming a resist film using the actinic ray-sensitive or radiation-sensitive resin composition according to any one of [1] to [11];

exposing the resist film to light; and

developing the exposed resist film using a developer.

[14] A method for manufacturing an electronic device, the method including the pattern forming method according to [13].

The present invention can provide an actinic ray-sensitive or radiation-sensitive resin composition capable of providing good resolution and good bridge margin. The present invention can also provide an actinic ray-sensitive or radiation-sensitive film using the actinic ray-sensitive or radiation-sensitive resin composition, a pattern forming method, and a method for manufacturing an electronic device.

### DESCRIPTION OF THE PREFERRED EMBODIMENTS

The present invention will next be described in detail.

Description of structural requirements described below may be made on the basis of representative embodiments of the present invention. However, the invention is not limited to theses embodiments.

In the present specification, “actinic rays” or “radiation” means, for example, an emission line spectrum of a mercury lamp, far-ultraviolet rays typified by excimer laser light, extreme ultraviolet light (EUV light), X-rays, soft X rays, an electron beam (EB), etc. In the present specification, “light” means actinic rays or radiation. In the present specification, “exposure to light” is intended to encompass not only exposure to an emission line spectrum of a mercury lamp, far-ultraviolet rays typified by excimer laser light, X-rays, EUV, etc. but also image drawing using an electron beam or a particle beam such as an ion beam.

In the present specification, “to” is used to mean that numerical values before and after the “to” are used as the lower limit and the upper limit.

In the present specification, no limitation is imposed on the bonding direction of a divalent group, unless otherwise specified. For example, when Y in a compound represented by a general formula “X—Y—Z” is —COO—, Y may be —CO—O— or may be —O—CO—. This compound may be “X—CO—O—Z” or may be “X—O—CO—Z.”

In the present specification, (meth)acrylate represents at least one of acrylate or methacrylate, and (meth)acrylic acid represents at least one of acrylic acid or methacrylic acid.

In the present specification, the weight average molecular weight (Mw), number average molecular weight (Mn), and dispersity (which may be referred to also as the “molecular weight distribution”) (Mw/Mn) of a resin are defined as polystyrene-equivalent values determined by GPC (Gel Permeation Chromatography) measurement (solvent: tetrahydrofuran, flow rate (injection amount of a sample): 10 μL, columns: TSK gel Multipore HXL-M manufactured by TOSOH Corporation, column temperature: 40° C., flow velocity: 1.0 mL/minute, detector: differential refractive index detector) using a GPC apparatus (HLC-8120GPC manufactured by TOSOH Corporation).

As for notations of groups (atomic groups) in the present specification, a notation that is not specified as substituted and unsubstituted is intended to encompass groups having no substituent and groups having a substituent. For example, an “alkyl group” is intended to encompass not only an alkyl group having no substituent (an unsubstituted alkyl group) but also an alkyl group having a substituent (a substituted alkyl group).

In the present specification, the term “organic group” means a group including at least one carbon atom.

In the present specification, when the phrase “optionally having a substituent” or the phrase “may have a substituent” appears, no particular limitation is imposed on the type of substituent, the position of the substituent, and the number of substituents. The number of substituents may be, for example, 1, 2, 3, or more. Examples of the substituent include monovalent nonmetallic atomic groups except for a hydrogen atom, and the substituent can be selected from, for example, the following substituents T.

### (Substituents T)

Examples of the substituents T include: halogen atoms such as a fluorine atom, a chlorine atom, a bromine atom, and an iodine atom; alkoxy groups such as a methoxy group, an ethoxy group, and a tert-butoxy group; aryloxy groups such as a phenoxy group and a p-tolyloxy group; alkoxycarbonyl groups such as a methoxycarbonyl group, a butoxycarbonyl group, and a phenoxycarbonyl group; acyloxy groups such as an acetoxy group, a propionyloxy group, and a benzoyloxy group; acyl groups such as an acetyl group, a benzoyl group, an isobutyryl group, an acryloyl group, a methacryloyl group, and a methoxalyl group; alkylsulfanyl groups such as a methylsulfanyl group and a tert-butylsulfanyl group; arylsulfanyl groups such as a phenylsulfanyl group and a p-tolylsulfanyl group; alkyl groups (having, for example, 1 to 10 carbon atoms); cycloalkyl groups (having, for example, 3 to 20 carbon atoms); aryl groups (having, for example, 6 to 20 carbon atoms); heteroaryl groups; a hydroxy group; a carboxy group; a formyl group; a sulfo group; a cyano group; alkylaminocarbonyl groups; arylaminocarbonyl groups; sulfonamido groups; silyl groups; an amino group; monoalkylamino groups; dialkylamino groups; arylamino groups; a nitro group; and combinations thereof.

In the present specification, the acid dissociation constant (pKa) is the pKa in an aqueous solution and is specifically a value determined by computation using the following software package 1 based on a Hammett substituent constant and a database of known literature values. All pKa values in the present specification are values determined by computation using this software package.

Software package 1: Advanced Chemistry Development (ACD/Labs) Software V 8.14 for Solaris (1994-2007 ACD/Labs).

The pKa can also be determined by a molecular orbital calculation method. In one specific example of this method, H⁺ dissociation free energy in a solvent is computed based on a thermodynamic cycle to compute the pKa. In the present specification, the solvent used is generally water. When the pKa cannot be determined using water, DMSO (dimethyl sulfoxide) is used.

As for the method for computing the H⁺ dissociation free energy, the density functional theory (DFT), for example, can be used for the computation. Various other methods have been reported in literature etc., but the computation method is not limited thereto. There are a plurality of software applications capable of performing the DFT, and one example is Gaussian 16.

In the present specification, the pKa is a value determined by computation using the software package 1 based on the Hammett substituent constant and the database of known literature values as described above. When the pKa cannot be computed using this method, a value obtained using Gaussian 16 based on the DFT (density functional theory) is used.

### (Actinic Ray-Sensitive or Radiation-Sensitive Resin Composition)

The actinic ray-sensitive or radiation-sensitive resin composition of the invention (which may be referred to also as the “composition of the invention”) is an actinic ray-sensitive or radiation-sensitive resin composition including: a resin (A) whose polarity increases by the action of an acid; and an acidic compound (F) having an iodine atom,
wherein the resin (A) is a different compound from the acidic compound (F),
wherein the acidic compound (F) is a nonionic compound, and
wherein the resin (A) has at least one selected from the group consisting of a repeating unit represented by general formula (3) below, a repeating unit represented by general formula (6) below, and a repeating unit represented by general formula (7) below:

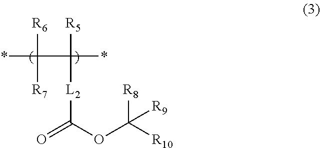

In general formula (3),

R₅ to R₇ each independently represent a hydrogen atom, an alkyl group, a cycloalkyl group, a halogen atom, a cyano group, or an alkoxycarbonyl group.

L₂ represents a divalent linking group.

R₈ to R₁₀ each independently represent an alkyl group, a cycloalkyl group, an aryl group, an aralkyl group, or an alkenyl group. Two selected from the group consisting of R₈ to R₁₀ may be bonded together to form a ring.

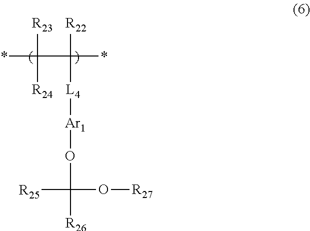

In general formula (6),

R₂₂ to R₂₄ each independently represent a hydrogen atom, an alkyl group, a cycloalkyl group, a halogen atom, a cyano group, or an alkoxycarbonyl group.

L₄ represents a single bond or a divalent linking group.

Ar₁ represents an aromatic group.

R₂₅ to R₂₇ each independently represent a hydrogen atom, an alkyl group, a cycloalkyl group, an aryl group, an aralkyl group, or an alkenyl group.

R₂₆ and R₂₇ may be bonded together to form a ring.

R₂₄ or R₂₅ may be bonded to Ar₁.

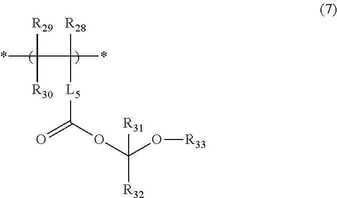

In general formula (7),

R₂₈ to R₃₀ each independently represent a hydrogen atom, an alkyl group, a cycloalkyl group, a halogen atom, a cyano group, or an alkoxycarbonyl group.

L₅ represents a single bond or a divalent linking group.

R₃₁ and R₃₂ each independently represent a hydrogen atom, an alkyl group, a cycloalkyl group, an aryl group, an aralkyl group, or an alkenyl group.

R₃₃ represents an alkyl group, a cycloalkyl group, an aryl group, an aralkyl group, or an alkenyl group.

R₃₂ and R₃₃ may be bonded together to form a ring.

The composition of the invention is preferably a resist composition and may be a positive-type resist composition or may be a negative-type resist composition. The composition of the invention may be a resist composition for alkali development or may be a resist composition for organic solvent development.

The composition of the invention is preferably a positive-type resist composition.

The composition of the invention is preferably a resist composition for alkali development.

The composition of the invention is preferably a chemical amplification-type resist composition and more preferably a chemical amplification positive-type resist composition.

Conventionally, when a resist composition is exposed to an electron beam or EUV, deterioration in bridge margin occurs in a high exposure dose region. This may be because of a local reduction in solubility caused by a side reaction due to secondary electron emission from a resin whose polarity increases by the action of an acid upon irradiation with an electron beam or EUV and is an essential problem.

The inventors have tried to improve the bridge margin by using an acidic compound (F) having an iodine atom and found that, when a repeating unit selected from the group consisting of the highly reactive repeating unit represented by general formula (3), the highly reactive repeating unit represented by general formula (6), and the highly reactive repeating unit represented by general formula (7) is used, both good resolution and good bridge margin can be achieved.

The reason that the composition of the invention can provide good resolution and good bridge margin is not completely clear, but the inventors have inferred that the reason is as follows.

The acidic compound (F) has an iodine atom and can easily absorb an electron beam or EUV. When added to the composition, the acidic compound (F) may efficiently emit secondary electrons instead of the resin whose polarity increases by the action of an acid. Therefore, the side reaction that occurs conventionally in the resin whose polarity increases by the action of an acid can be prevented, and the bridge margin may thereby be improved.

Since the acidic compound (F) is acidic, its solubility in a developer is high. This may also be a factor in preventing a reduction in local solubility due to the side reaction.

Moreover, when the acidic compound (F) is used in combination with a highly reactive protective group, the effect of improving resolution is obtained. This may be because, although the highly reactive protective group has the potential to provide high resolution, its performance is not fully demonstrated because of the side reaction of the resin whose polarity increases by the action of an acid.

### [Resin (A) Whose Polarity Increases by Action of Acid]

The resin whose polarity increases by the action of an acid (which is referred to also as the “resin (A)”) will be described.

### <Repeating Unit Having Acid-Decomposable Group>

The resin (A) is a resin that is decomposed by the action of an acid and thereby increased in polarity.

The resin (A) has a repeating unit that is decomposed by the action of an acid and thereby increased in polarity (which is referred to also as an “acid-decomposable group”).

The resin (A) is increased in polarity by the action of an acid. In this case, the degree of solubility of the resin (A) in an alkali developer increases, and the degree of solubility in an organic solvent decreases.

When a pattern is formed using the composition of the invention including the resin (A), a positive-type pattern is typically formed when the developer used is an alkali developer, and a negative-type pattern is formed when the developer used is an organic-based developer.

Preferably, the acid-decomposable group is a group that is decomposed by the action of an acid to generate a polar group. Preferably, the acid-decomposable group has a structure in which the polar group is protected by a leaving group that leaves by the action of an acid. Specifically, it is preferable that the resin (A) has a repeating unit having a group that is decomposed by the action of an acid to generate a polar group.

The polar group is preferably an alkali-soluble group, and examples thereof include: acidic groups such as a carboxy group, phenolic hydroxy groups, fluorinated alcohol groups, sulfonic acid groups, phosphoric acid groups, sulfonamido groups, sulfonylimido groups, (alkylsulfonyl)(alkylcarbonyl)methylene groups, (alkylsulfonyl)(alkylcarbonyl)imido groups, bis(alkylcarbonyl)methylene groups, bis(alkylcarbonyl)imido groups, bis(alkylsulfonyl)methylene groups, bis(alkylsulfonyl)imido groups, tris(alkylcarbonyl)methylene groups, and tris(alkylsulfonyl)methylene groups; and alcoholic hydroxyl groups.

The polar group is preferably a carboxy group, a phenolic hydroxy group, a fluorinated alcohol group (preferably a hexafluoroisopropanol group), or a sulfonic acid group and more preferably a carboxy group or a phenolic hydroxy group. Specifically, the acid-decomposable group is preferably a group that is decomposed by the action of an acid to thereby generate a carboxy group or a group that is decomposed by the action of an acid to thereby generate a phenolic hydroxy group.

Preferably, the resin (A) has a repeating unit having at least one acid-decomposable group selected from the group consisting of a group that is decomposed by the action of an acid to generate a carboxy group and a group that is decomposed by the action of an acid to generate a phenolic hydroxy group.

Examples of the leaving group that leaves by the action of an acid include groups represented by formulas (Y1) to (Y4).

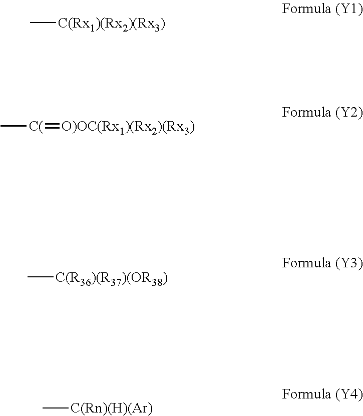

In formulas (Y1) and (Y2), Rx₁ to Rx₃ each independently represent an alkyl group (linear or branched alkyl group), a cycloalkyl group (monocyclic or polycyclic cycloalkyl group), an aryl group (monocyclic or polycyclic aryl group), an aralkyl group (linear or branched aralkyl group), or an alkenyl group (linear or branched alkenyl group). When all of Rx₁ to Rx₃ are alkyl groups (linear or branched alkyl groups), it is preferable that at least two selected from the group consisting of Rx₁ to Rx₃ are each a methyl group.

In particular, it is preferable that Rx₁ to Rx₃ each independently represent a linear or branched alkyl group, and it is more preferable that Rx₁ to Rx₃ each independently represent a linear alkyl group.

Two selected from the group consisting of Rx₁ to Rx₃ may be bonded together to form a ring (which may be a monocyclic ring or a polycyclic ring).

The alkyl group represented by each of Rx₁ to Rx₃ is preferably an alkyl group having 1 to 5 carbon atoms such as a methyl group, an ethyl group, a n-propyl group, an isopropyl group, a n-butyl group, an isobutyl group, or a t-butyl group.

The cycloalkyl group represented by each of Rx₁ to Rx₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

The aryl group represented by each of Rx₁ to Rx₃ is preferably an aryl group having 6 to 10 carbon atoms, and examples thereof include a phenyl group, a naphthyl group, and an anthryl group.

The aralkyl group represented by each of Rx₁ to Rx₃ is preferably a group formed by replacing one hydrogen atom in the alkyl group represented by any of Rx₁ to Rx₃ with an aryl group having 6 to 10 carbon atoms (preferably a phenyl group), and examples thereof include a benzyl group.

The alkenyl group represented by each of Rx₁ to Rx₃ is preferably a vinyl group.

The ring formed by bonding two selected from the group consisting of Rx₁ to Rx₃ is preferably a cycloalkyl group. The cycloalkyl group formed by bonding two selected from the group consisting of Rx₁ to Rx₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group and is more preferably a monocyclic cycloalkyl group having 5 to 6 carbon atoms.

For example, one methylene group included in the ring in the cycloalkyl group formed by bonding two selected from the group consisting of Rx₁ to Rx₃ may be replaced with a heteroatom such as an oxygen atom, a group having a heteroatom such as a carbonyl group, or a vinylidene group. In any of these cycloalkyl groups, at least one ethylene group included in the cycloalkane ring may be replaced with a vinylene group.

In the group represented by formula (Y1) or formula (Y2), it is preferable that, for example, Rx₁ is a methyl group or an ethyl group and that Rx₂ and Rx₃ are bonded together to form the cycloalkyl group described above.

In formula (Y3), R₃₆ to R₃₈ each independently represent a hydrogen atom or a monovalent organic group. R₃₇ and R₃₈ may be bonded together to form a ring. Examples of the monovalent organic group include alkyl groups, cycloalkyl groups, aryl groups, aralkyl groups, and alkenyl groups. It is also preferable that R₃₆ is a hydrogen atom.

The alkyl, cycloalkyl, aryl, and aralkyl groups described above may each include a heteroatom such as an oxygen atom and/or a group having a heteroatom such as a carbonyl group. For example, in the alkyl, cycloalkyl, aryl, and aralkyl groups described above, at least one methylene group may be replaced with a heteroatom such as an oxygen atom and/or a group having a heteroatom such as a carbonyl group.

R₃₈ may be bonded to another substituent included in the main chain of the repeating unit to form a ring. The group formed by bonding R₃₈ and another substituent included in the main chain of the repeating unit is preferably an alkylene group such as a methylene group.

Formula (Y3) is preferably a group represented by the following formula (Y3-1).

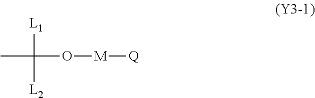

L₁ and L₂ each independently represent a hydrogen atom, an alkyl group, a cycloalkyl group, an aryl group, or a group formed by combining any of them (for example, a group formed by combining an alkyl group and an aryl group).

M represents a single bond or a divalent linking group.

Q represents an alkyl group optionally including a heteroatom, a cycloalkyl group optionally including a heteroatom, an aryl group optionally including a heteroatom, an amino group, an ammonium group, a mercapto group, a cyano group, an aldehyde group, or a group formed by combining any of them (for example, a group formed by combining an alkyl group and a cycloalkyl group).

In the alkyl and cycloalkyl groups, for example, one methylene group may be replaced with a heteroatom such as an oxygen atom or a group having a heteroatom such as a carbonyl group.

It is preferable that one of L₁ or L₂ is a hydrogen atom and that the other is an alkyl group, a cycloalkyl group, an aryl group, or a group formed by combining an alkylene group and an aryl group.

At least two selected from the group consisting of Q, M, and L₁ may be bonded together to form a ring (preferably a 5-membered or 6-membered ring).

From the viewpoint of obtaining a finer pattern, L₂ is preferably a secondary or tertiary alkyl group and more preferably a tertiary alkyl group. Examples of the secondary alkyl group include an isopropyl group, a cyclohexyl group, and a norbornyl group, and examples of the tertiary alkyl group include a tert-butyl group and an adamantane group. In these modes, since Tg (glass transition temperature) and activation energy are high, high film hardness is obtained, and the occurrence of fogging can be reduced.

In formula (Y4), Ar represents an aromatic ring group. Rn represents an alkyl group, a cycloalkyl group, or an aryl group. Rn and Ar may be bonded together to form a non-aromatic ring. Ar is more preferably an aryl group.

When, in the leaving group protecting the polar group, a non-aromatic ring is bonded directly to the polar group (or its residue), it is also preferable that a ring member atom adjacent to the ring member atom bonded directly to the polar group (or its residue) in the non-aromatic ring does not have a halogen atom such as a fluorine atom as a substituent, because the repeating unit can have good acid-decomposability.

The leaving group that leaves by the action of an acid may also be a 2-cyclopentenyl group having a substituent (e.g., an alkyl group) such as a 3-methyl-2-cyclopentenyl group or a cyclohexyl group having a substituent (e.g., an alkyl group) such as a 1,1,4,4-tetramethylcyclohexyl group.

The resin (A) has at least one selected from the group consisting of a repeating unit represented by general formula (3) below, a repeating unit represented by general formula (6) below, and a repeating unit represented by general formula (7) below.

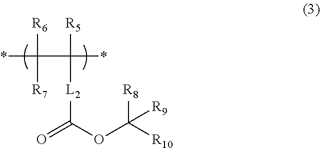

In general formula (3),

R₅ to R₇ each independently represent a hydrogen atom, an alkyl group, a cycloalkyl group, a halogen atom, a cyano group, or an alkoxycarbonyl group.

L₂ represents a divalent linking group.

R₈ to R₁₀ each independently represent an alkyl group, a cycloalkyl group, an aryl group, an aralkyl group, or an alkenyl group. Two selected from the group consisting of R₈ to R₁₀ may be bonded together to form a ring.

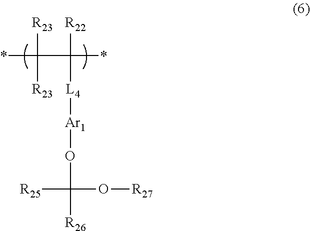

In general formula (6),

R₂₂ to R₂₄ each independently represent a hydrogen atom, an alkyl group, a cycloalkyl group, a halogen atom, a cyano group, or an alkoxycarbonyl group.

L₄ represents a single bond or a divalent linking group.

Ar₁ represents an aromatic group.

R₂₅ to R₂₇ each independently represent a hydrogen atom, an alkyl group, a cycloalkyl group, an aryl group, an aralkyl group, or an alkenyl group.

R₂₆ and R₂₇ may be bonded together to form a ring.

R₂₄ or R₂₅ may be bonded to Ar₁.

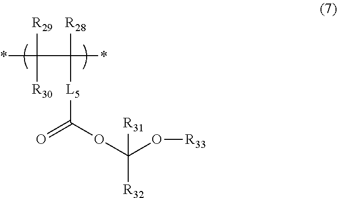

In general formula (7),

R₂₈ to R₃₀ each independently represent a hydrogen atom, an alkyl group, a cycloalkyl group, a halogen atom, a cyano group, or an alkoxycarbonyl group.

L₅ represents a single bond or a divalent linking group.

R₃₁ and R₃₂ each independently represent a hydrogen atom, an alkyl group, a cycloalkyl group, an aryl group, an aralkyl group, or an alkenyl group.

R₃₃ represents an alkyl group, a cycloalkyl group, an aryl group, an aralkyl group, or an alkenyl group.

R₃₂ and R₃₃ may be bonded together to form a ring.

The repeating unit represented by general formula (3) will be described.

The repeating unit represented by general formula (3) is a repeating unit having an acid-decomposable group.

The alkyl group represented by each of R₅, R₆, and R₇ may be linear or branched. No particular limitation is imposed on the number of carbon atoms in the alkyl group, but the number of carbon atoms is preferably 1 to 5 and more preferably 1 to 3.

The cycloalkyl group represented by each of R₅, R₆, and R₇ is preferably a monocyclic cycloalkyl group such as a cyclopentyl group or cyclohexyl group or a polycyclic cycloalkyl group such as a norbornyl group, a tetracyclodecanyl group, a tetracyclododecanyl group, or an adamantyl group.

Examples of the halogen atom represented by each of R₅, R₆, and R₇ include a fluorine atom, a chlorine atom, a bromine atom, and an iodine atom, and the halogen atom is preferably a fluorine atom or an iodine atom.

The alkyl group included in the alkoxycarbonyl group represented by each of R₅, R₆, and R₇ may be linear or branched. No particular limitation is imposed on the number of carbon atoms in the alkyl group included in the alkoxycarbonyl group, but the number of carbon atoms is preferably 1 to 5 and more preferably 1 to 3.

Examples of the divalent linking group represented by L₂ include —CO—, —O—, —S—, —SO—, —SO₂—, hydrocarbon groups (such as alkylene groups, cycloalkylene groups, alkenylene groups, and arylene groups), and linking groups formed by linking any of the above groups.

The alkyl group represented by each of R₈ to R₁₀ may be linear or branched. No particular limitation is imposed on the number of carbon atoms in the alkyl group, but the number of carbon atoms is preferably 1 to 5 and more preferably 1 to 3. In the alkyl group represented by each of R₈ to R₁₀, a methylene group may be replaced with at least one of —CO— or —O—.

The cycloalkyl group represented by each of R₈ to R₁₀ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

The aryl group represented by each of R₈ to R₁₀ is preferably a phenyl group.

The aralkyl group represented by each of R₈ to R₁₀ is preferably a group formed by replacing one hydrogen atom in the alkyl group represented by any of R₈ to R₁₀ with an aryl group having 6 to 10 carbon atoms (preferably a phenyl group), and examples thereof include a benzyl group.

The alkenyl group represented by each of R₈ to R₁₀ is preferably a vinyl group.

The group formed by bonding two selected from the group consisting of R₈ to R₁₀ together is preferably a cycloalkyl group.

The cycloalkyl group formed by bonding two selected from the group consisting of R₈ to R₁₀ together is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group and is more preferably a monocyclic cycloalkyl group having 5 to 6 carbon atoms.

In the cycloalkyl group formed by bonding two selected from the group consisting of R₈ to R₁₀ together, for example, one methylene group included in the ring may be replaced with a heteroatom such as an oxygen atom, a group having a heteroatom such as a carbonyl group, or a vinylidene group. In the cycloalkyl group, at least one ethylene group included in the cycloalkane ring may be replaced with a vinylene group.

Each group in general formula (3) may have a substituent, and examples of the substituent include the substituents T described above.

The repeating unit represented by general formula (6) will be described.

The repeating unit represented by general formula (6) is a repeating unit having an acid-decomposable group.

R₂₂, R₂₃, and R₂₄ have the same meanings as R₅, R₆, and R₇ in general formula (3), and their preferred ranges are also the same as those of R₅, R₆, and R₇.

When L₄ represents a divalent linking group, examples of the divalent linking group include —CO—, —O—, —S—, —SO—, —SO₂—, hydrocarbon groups (such as alkylene groups, cycloalkylene groups, alkenylene groups, and arylene groups), and linking groups formed by linking any of these groups.

No particular limitation is imposed on the aromatic group represented by Ar₁, but examples thereof include a phenylene group and a naphthylene group.

Examples of the alkyl, cycloalkyl, aryl, aralkyl, and alkenyl groups represented by R₂₅ to R₂₇ include the same groups as those for the alkyl, cycloalkyl, aryl, aralkyl, and alkenyl groups represented by R₈ to R₁₀ in general formula (3) described above.

The alkyl, cycloalkyl, aryl, aralkyl, and alkenyl groups represented by R₂₅ to R₂₇ may each have a substituent, and examples of the substituent include the substituents T described above.

The ring formed by bonding R₂₆ and R₂₇, the ring formed by bonding Ar and R₂₄, and the ring formed by bonding R₂₅ and Ar are each preferably a cycloalkyl group. The cycloalkyl group formed by bonding R₂₆ and R₂₇, the cycloalkyl group formed by bonding Ar and R₂₄, and the cycloalkyl group formed by bonding R₂₅ and Ar₁ are each preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group and more preferably a monocyclic cycloalkyl group having 5 to 6 carbon atoms.

In the cycloalkyl group formed by bonding R₂₆ and R₂₇, the cycloalkyl group formed by bonding Ar and R₂₄, and the cycloalkyl group formed by bonding R₂₅ and Ar₁, for example, one methylene group included in the ring may be replaced with a heteroatom such as an oxygen atom, a group having a heteroatom such as a carbonyl group, or a vinylidene group. In these cycloalkyl groups, at least one ethylene group included in the cycloalkane ring may be replaced with a vinylene group.

The repeating unit represented by general formula (7) will be described.

The repeating unit represented by general formula (7) is a repeating unit having an acid-decomposable group.

R₂₈, R₂₉, R₃₀, and L₅ have the same meanings as R₂₂, R₂₃, R₂₄, and L₄ in general formula (6), and their preferred ranges are also the same as those of R₂₂, R₂₃, R₂₄, and L₄.

Examples of the alkyl, cycloalkyl, aryl, aralkyl, and alkenyl groups represented by R₃₁, R₃₂, and R₃₃ include the same groups as those for the alkyl, cycloalkyl, aryl, aralkyl, and alkenyl groups represented by R₈ to R₁₀ in general formula (3).

The alkyl, cycloalkyl, aryl, aralkyl, and alkenyl groups represented by R₃₁, R₃₂, and R₃₃ may each have a substituent, and examples thereof include the substituents T described above.

The ring formed by bonding R₃₂ and R₃₃ is preferably a cycloalkyl group. The cycloalkyl group formed by bonding R₃₂ and R₃₃ is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group and is more preferably a monocyclic cycloalkyl group having 5 to 6 carbon atoms.

In the cycloalkyl group formed by bonding R₃₂ and R₃₃, for example, one methylene group included in the ring may be replaced with a heteroatom such as an oxygen atom, a group having a heteroatom such as a carbonyl group, or a vinylidene group. In the cycloalkyl group, at least one ethylene group included in the cycloalkane ring may be replaced with a vinylene group.

The repeating unit selected from the group consisting of the repeating unit represented by general formula (3), the repeating unit represented by general formula (6), and the repeating unit represented by general formula (7) may or may not include a halogen atom but preferably includes no halogen atom.

The content of the repeating unit selected from the group consisting of the repeating unit represented by general formula (3), the repeating unit represented by general formula (6), and the repeating unit represented by general formula (7) (the total content when a plurality of repeating units are included) with respect to the total amount of the repeating units in the resin (A) is preferably 5% by mole or more, more preferably 10% by mole or more, still more preferably 15% by mole or more, particularly preferably 20% by mole or more, and most preferably 25% by mole or more.

The content of the repeating unit selected from the group consisting of the repeating unit represented by general formula (3), the repeating unit represented by general formula (6), and the repeating unit represented by general formula (7) (the total content when a plurality of repeating units are included) with respect to the total amount of the repeating units in the resin (A) is preferably 95% by mole or less, more preferably 90% by mole or less, still more preferably 85% by mole or less, particularly preferably 80% by mole or less, and most preferably 75% by mole or less.

Specific examples of the repeating unit represented by general formula (3) are shown below, but the repeating unit is not limited thereto.

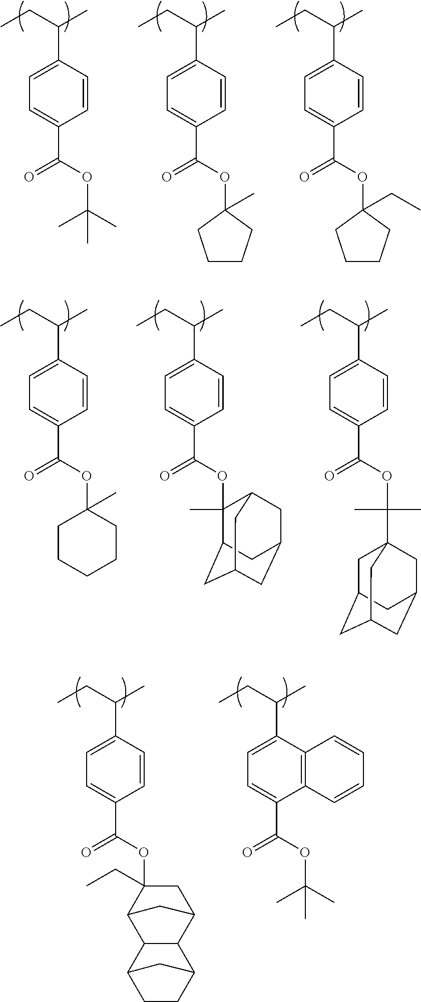

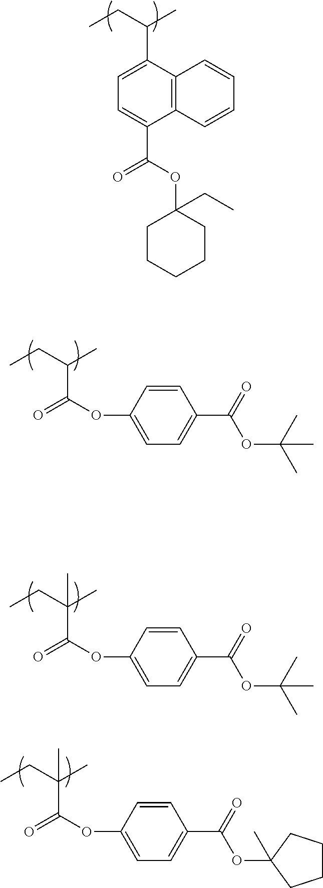

Specific examples of the repeating unit represented by general formula (6) are shown below, but the repeating unit is not limited thereto.

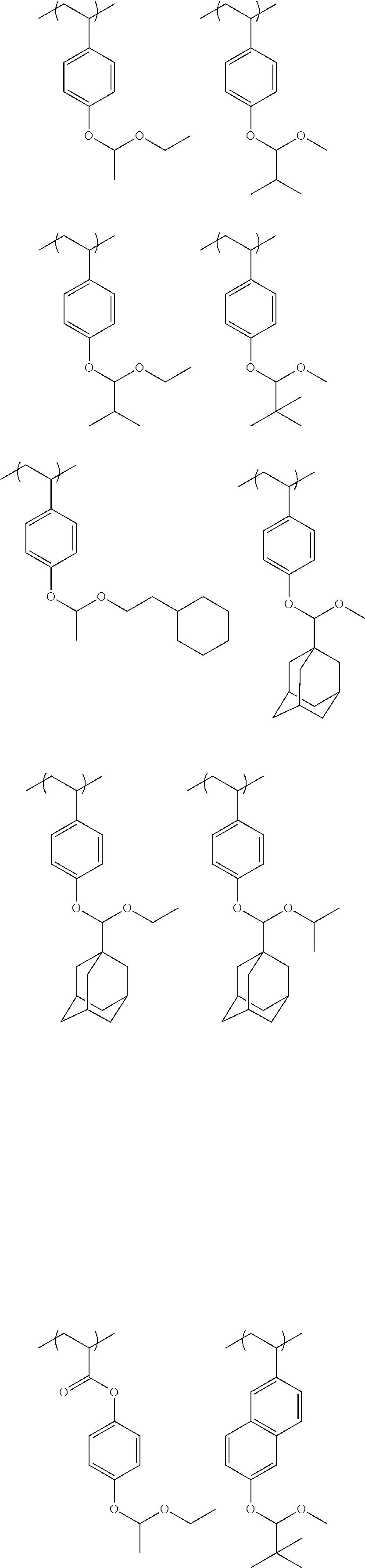

Specific examples of the repeating unit represented by general formula (7) are shown below, but the repeating unit is not limited thereto. In the following structural formulas, Xa₁ represents any of H, CH₃, CF₃, and CH₂OH, and Rxa represents a linear or branched alkyl group having 1 to 5 carbon atoms.

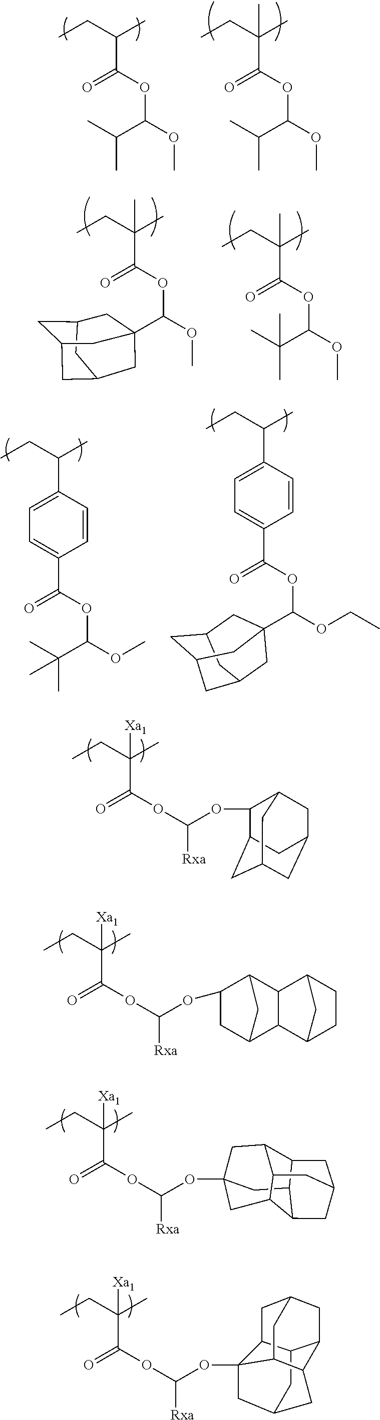

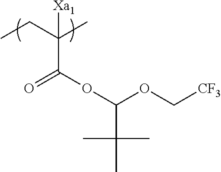

The resin (A) may include an additional repeating unit having an acid-decomposable group in addition to the repeating unit selected from the group consisting of the repeating unit represented by general formula (3), the repeating unit represented by general formula (6), and the repeating unit represented by general formula (7). When the resin (A) has the additional repeating unit having an acid-decomposable group, the content of the additional repeating unit having an acid-decomposable group (the total content when a plurality of additional repeating units each having an acid-decomposable group are included) with respect to the total amount of the repeating units in the resin (A) is preferably 1% by mole or more and 50% by mole or less and more preferably 3% by mole or more and 40% by mole or less.

Specific examples of the additional repeating unit having an acid-decomposable group other than the repeating unit selected from the group consisting of the repeating unit represented by general formula (3), the repeating unit represented by general formula (6), and the repeating unit represented by general formula (7) are shown below, but the repeating unit is not limited thereto. In the following structural formulas, Xa₁ represents any of H, CH₃, CF₃, and CH₂OH, and Rxa represents a linear or branched alkyl group having 1 to 5 carbon atoms.

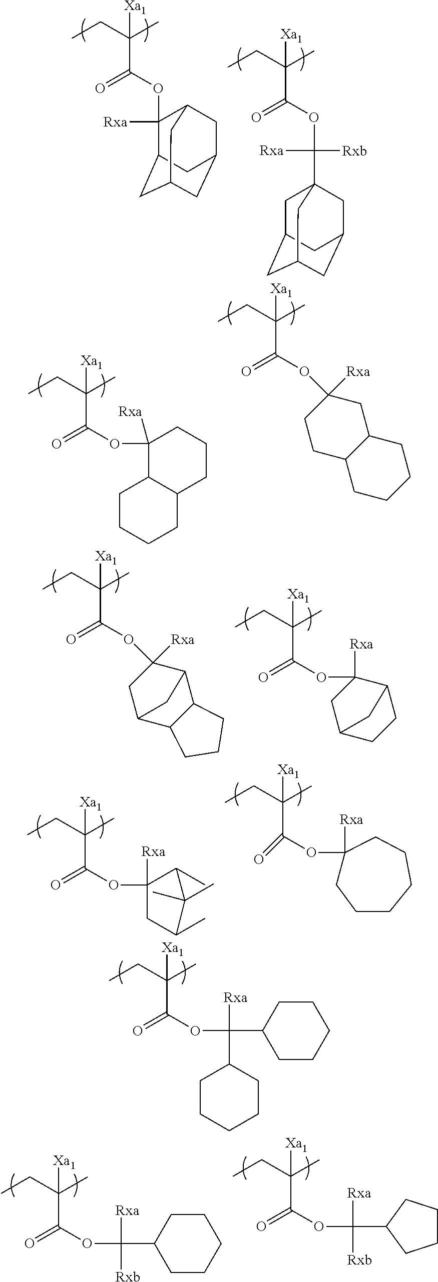

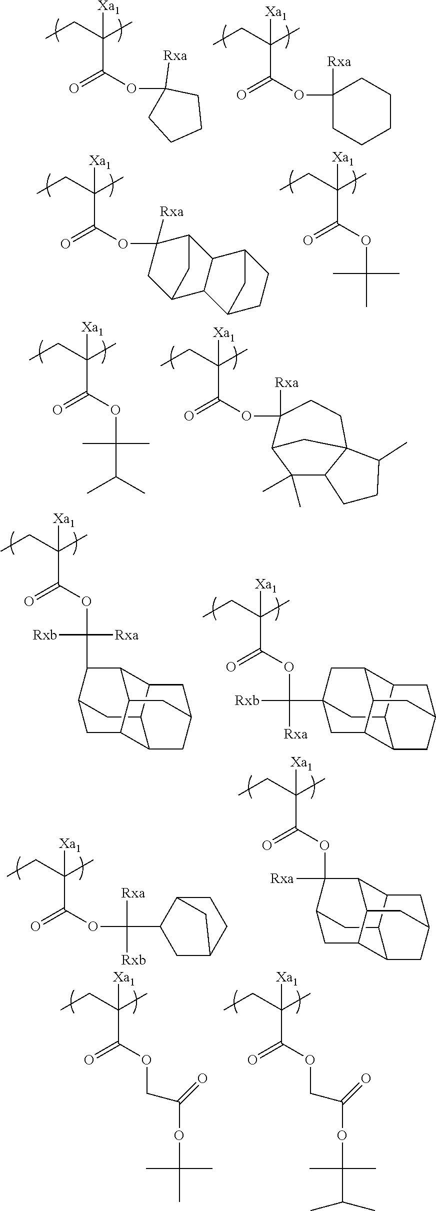

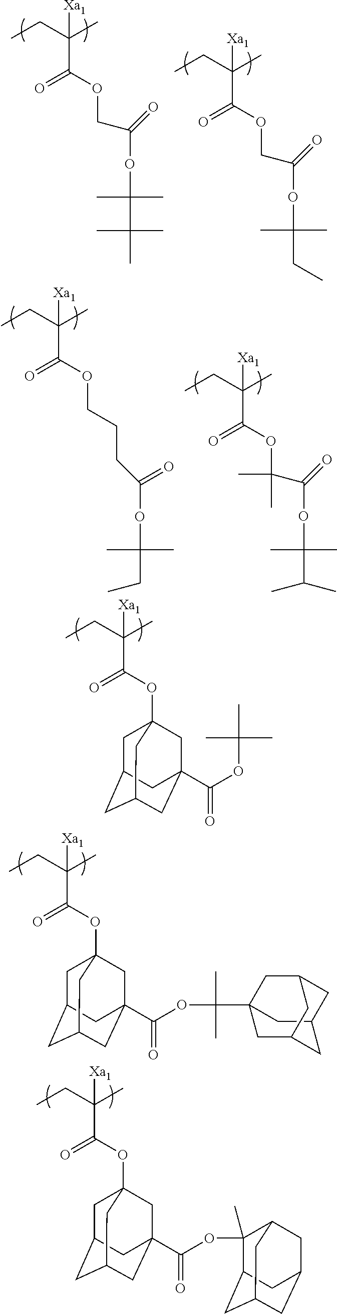

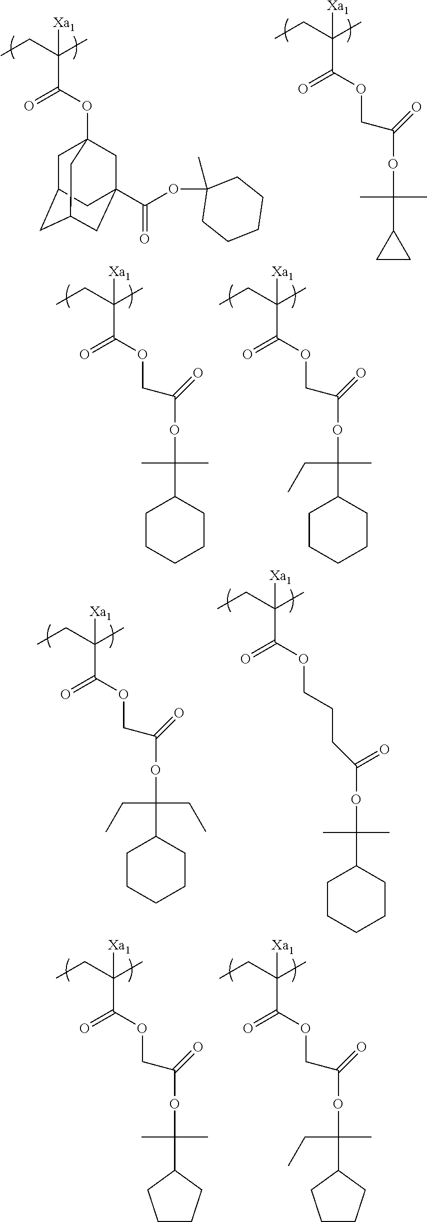

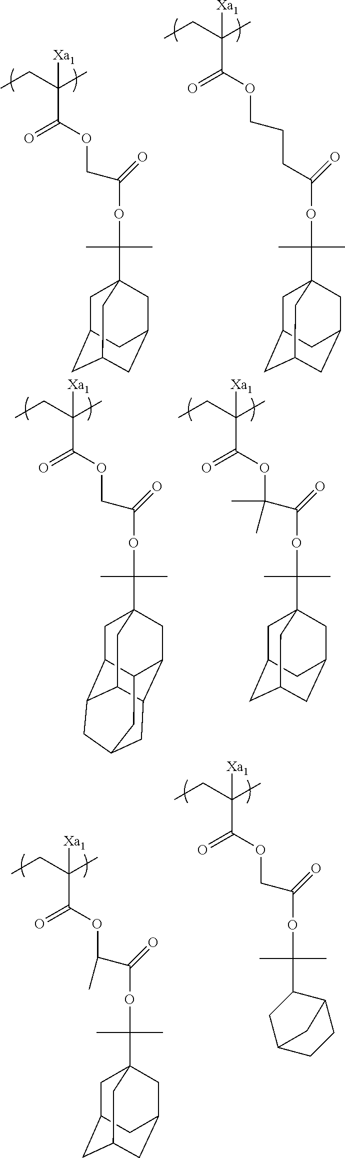

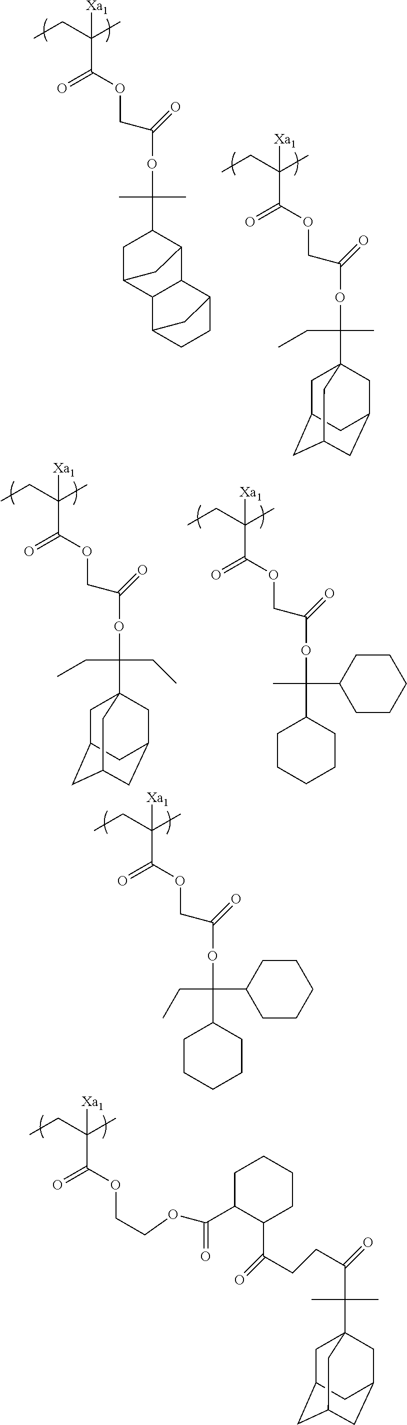

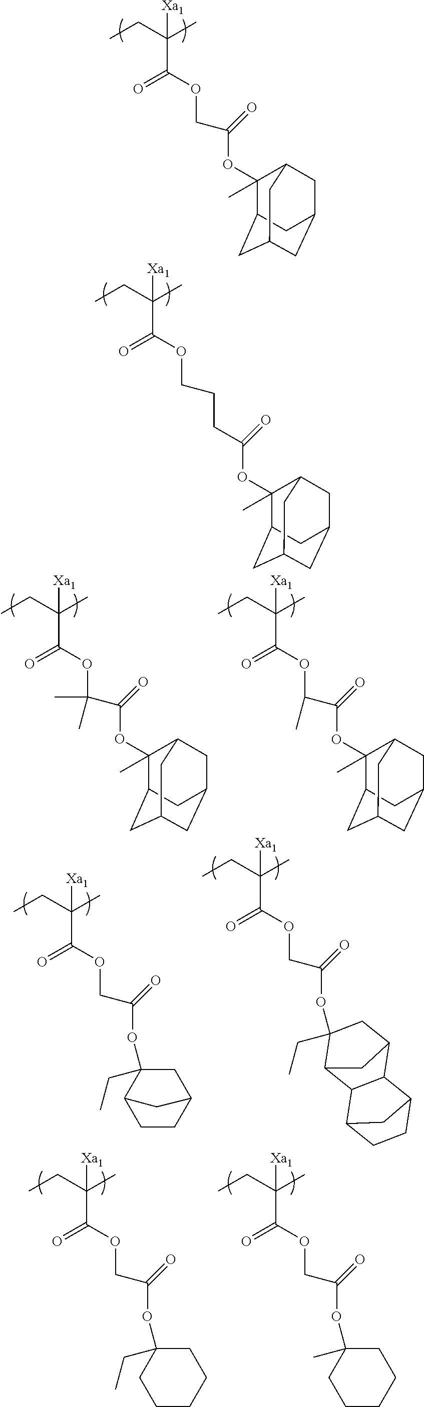

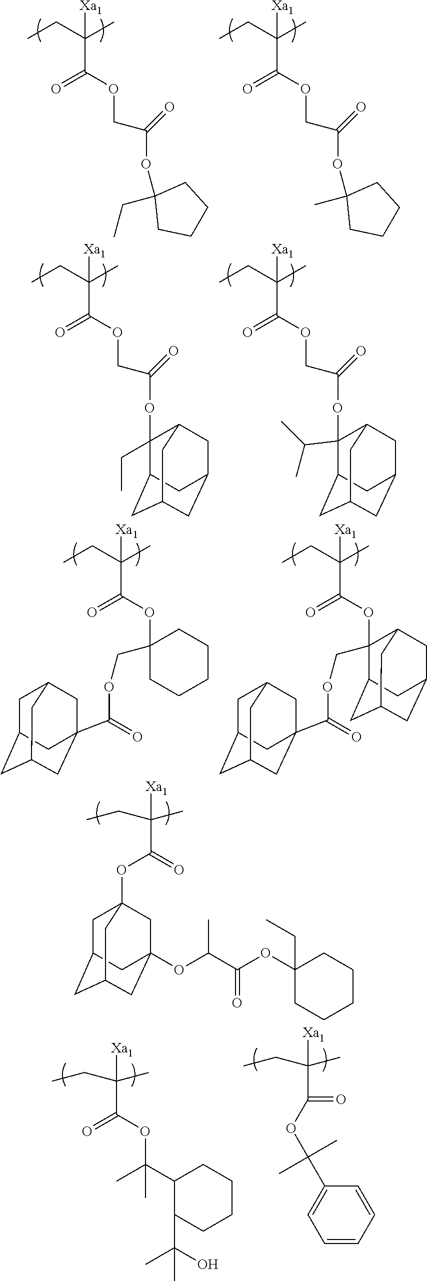

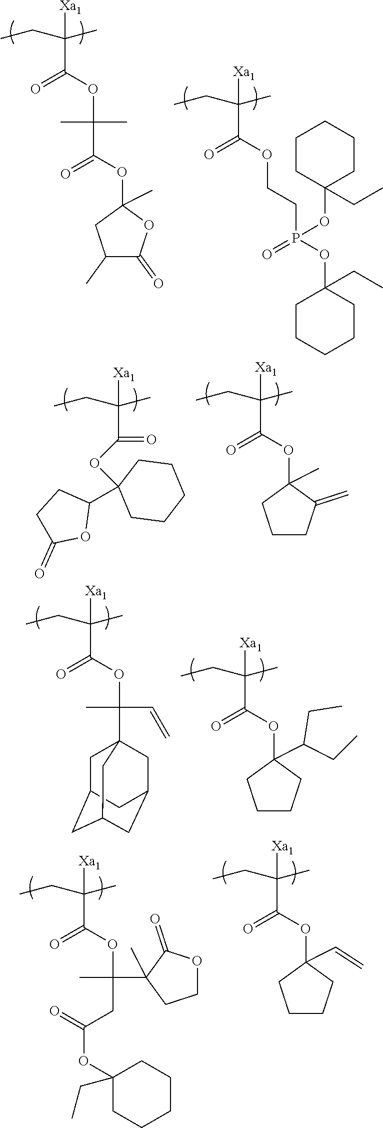

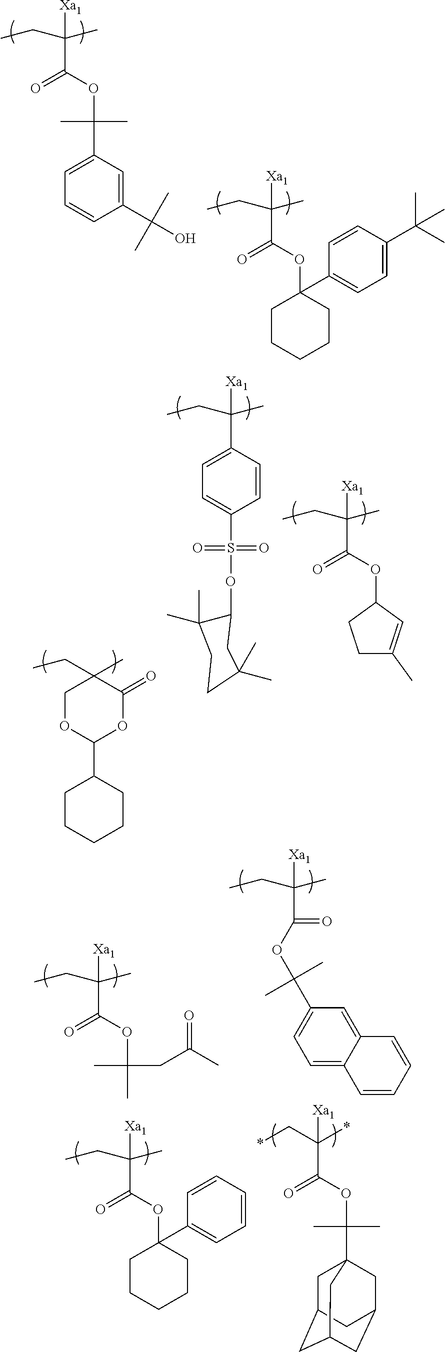

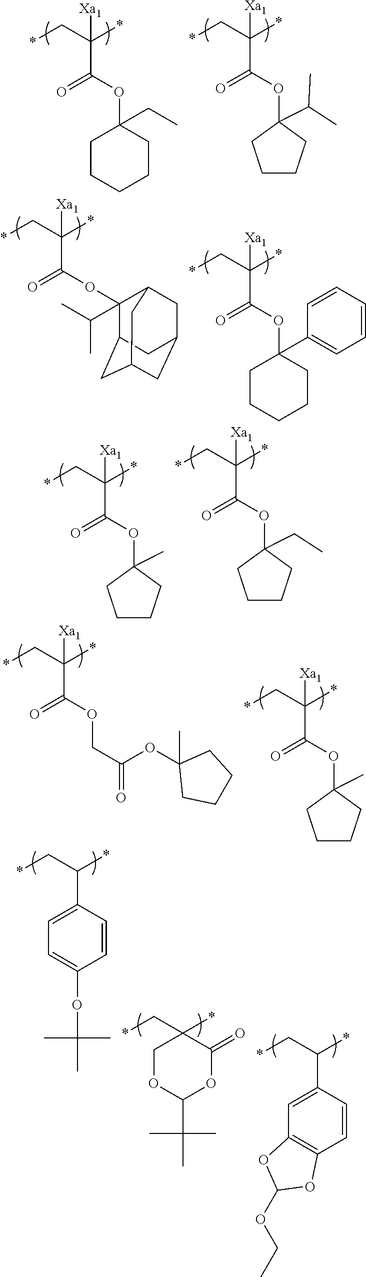

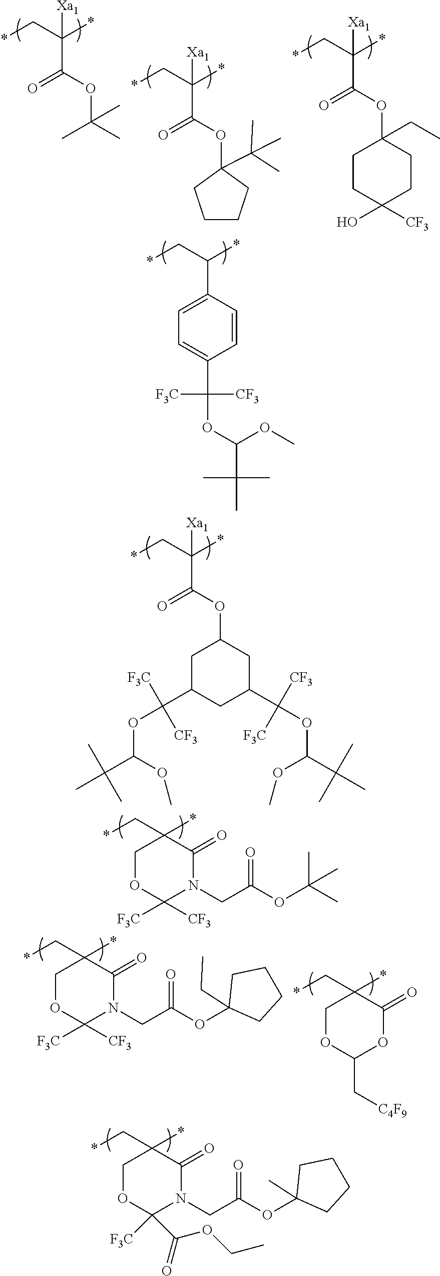

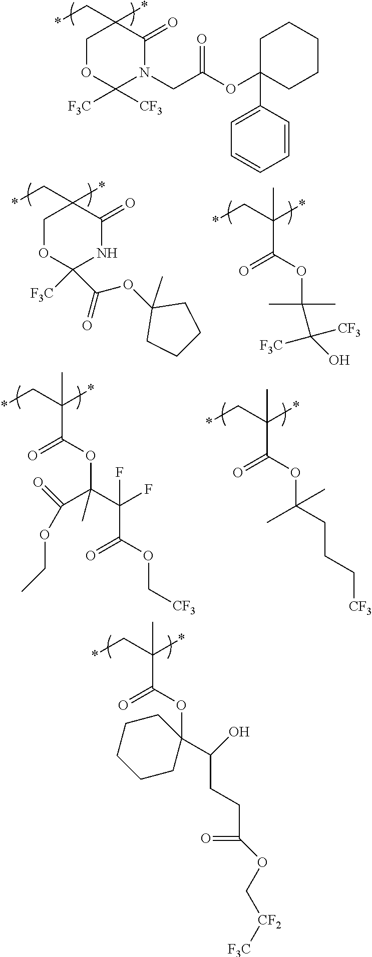

### <Repeating Unit Represented by General Formula (A2)>

Preferably, the resin (A) includes a repeating unit represented by the following general formula (A2).

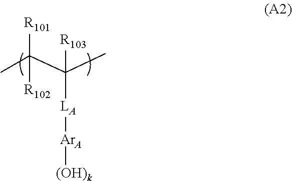

In general formula (A2),

R₁₀₁, R₁₀₂, and R₁₀₃ each independently represent a hydrogen atom, an alkyl group, a cycloalkyl group, a halogen atom, a cyano group, or an alkoxycarbonyl group.

L_{A} represents a single bond or a divalent linking group.

Ar_{A} represents an aromatic group.

k represents an integer of 1 to 5.

R₁₀₂ and Ar_{A} may be bonded together. When R₁₀₂ and Ar_{A} are bonded together, R₁₀₂ represents a single bond or an alkylene group.

When any of R₁₀₁, R₁₀₂, and R₁₀₃ in general formula (A2) represents an alkyl group, no particular limitation is imposed on the alkyl group. The alkyl group is preferably an alkyl group having 1 to 20 carbon atoms, more preferably an alkyl group having 1 to 8 carbon atoms, and still more preferably an alkyl group having 1 to 3 carbon atoms. Specific examples of the alkyl group include a methyl group, an ethyl group, a propyl group, an isopropyl group, a n-butyl group, a sec-butyl group, a hexyl group, a 2-ethylhexyl group, an octyl group, and a dodecyl group.

When any of R₁₀₁, R₁₀₂, and R₁₀₃ in general formula (A2) represents a cycloalkyl group, the cycloalkyl group may be monocyclic or may be polycyclic. The cycloalkyl group is preferably a monocyclic cycloalkyl group having 3 to 8 carbon atoms such as a cyclopropyl group, a cyclopentyl group, or a cyclohexyl group.

When any of R₁₀₁, R₁₀₂, and R₁₀₃ in general formula (A2) represents a halogen atom, examples of the halogen atom include a fluorine atom, a chlorine atom, a bromine atom, and an iodine atom, and the halogen atom is preferably a fluorine atom.

When any of R₁₀₁, R₁₀₂, and R₁₀₃ in general formula (A2) represents an alkoxycarbonyl group, specific examples of the alkyl group included in the alkoxycarbonyl group and its preferred range are the same as those of the alkyl group when any of R₁, R₂, and R₃ described above represents an alkyl group.

When the groups described above can each have at least one additional substituent, they may each have at least one additional substituent. No particular limitation is imposed on the additional substituent. Example of the additional substituent include alkyl groups, cycloalkyl groups, aryl groups, an amino group, amido groups, ureide groups, urethane groups, a hydroxy group, a carboxy group, halogen atoms, alkoxy groups, thioether groups, acyl groups, acyloxy groups, alkoxycarbonyl groups, a cyano group, and a nitro group. The number of carbon atoms in the additional substituent is preferably 8 or less.

R₁₀₁ and R₁₀₂ in general formula (A2) are each preferably a hydrogen atom.

R₁₀₃ in general formula (A2) is preferably a hydrogen atom or a methyl group and is more preferably a hydrogen atom.

Ar_{A} in general formula (A2) represents an aromatic group and is specifically a (k+1) valent aromatic group. The divalent aromatic group when k is 1 is, for example, preferably an arylene groups having 6 to 18 carbon atoms such as a phenylene group, a tolylene group, a naphthylene group, or an anthracenylene group or a heteroring-containing divalent aromatic group such as a thiophene ring, a furan ring, a pyrrole ring, a benzothiophene ring, a benzofuran ring, a benzopyrrole ring, a triazine ring, an imidazole ring, a benzimidazole ring, a triazole ring, a thiadiazole ring, or a thiazole ring. The aromatic group may have a substituent.

Specific examples of the (k+1) valent aromatic group when k is an integer of 2 or more include groups obtaining by removing (k−1) hydrogen atom(s) from the above-described specific examples of the aromatic group.

The (k+1) valent aromatic group may further have a substituent.

No particular limitation is imposed on the substituent that the (k+1) valent aromatic group can have. Examples of the substituent include: alkyl groups such as a methyl group, an ethyl group, a propyl group, an isopropyl group, a n-butyl group, a sec-butyl group, a hexyl group, a 2-ethylhexyl group, an octyl group, and a dodecyl group; alkoxy groups such as a methoxy group, an ethoxy group, a hydroxyethoxy group, a propoxy group, a hydroxypropoxy group, and a butoxy group; and aryl groups such as a phenyl group.

Ar_{A} represents preferably an aromatic group having 6 to 18 carbon atoms and represents more preferably a benzene ring group, a naphthalene ring group, or a biphenylene ring group.

L_{A} in general formula (A2) represents a single bond or a divalent linking group.

When L_{A} represents a divalent linking group, no particular limitation is imposed on the divalent linking group. Examples of the divalent linking group include —COO—, —CONR₆₄—, alkylene groups, and groups formed by combining two or more of them. R₆₄ represents a hydrogen atom or an alkyl group.

No particular limitation is imposed on the alkylene group, but the alkylene group is preferably an alkylene group having 1 to 8 carbon atoms such as a methylene group, an ethylene group, a propylene group, a butylene group, a hexylene group, or an octylene group.

When R₆₄ represents an alkyl group, examples of the alkyl group include alkyl groups having 20 or less carbon atoms such as a methyl group, an ethyl group, a propyl group, an isopropyl group, a n-butyl group, a sec-butyl group, a hexyl group, a 2-ethylhexyl group, an octyl group, and a dodecyl group. The alkyl group is preferably an alkyl group having 8 or less carbon atoms.

Preferably, the repeating unit represented by general formula (A2) has a hydroxy styrene structure. Specifically, it is preferable that Ar_{A} represents a benzene ring group.

k represents preferably an integer of 1 to 3 and represents more preferably 1 or 2.

Specific examples of the repeating unit represented by general formula (A2) are shown below. In the structural formulas in the specific examples, a represents 1, 2, or 3. As for specific examples of the repeating unit represented by general formula (A2), reference can be made to the description in paragraphs [0068] to [0072] of WO2018/193954A, the contents of which are incorporated herein.

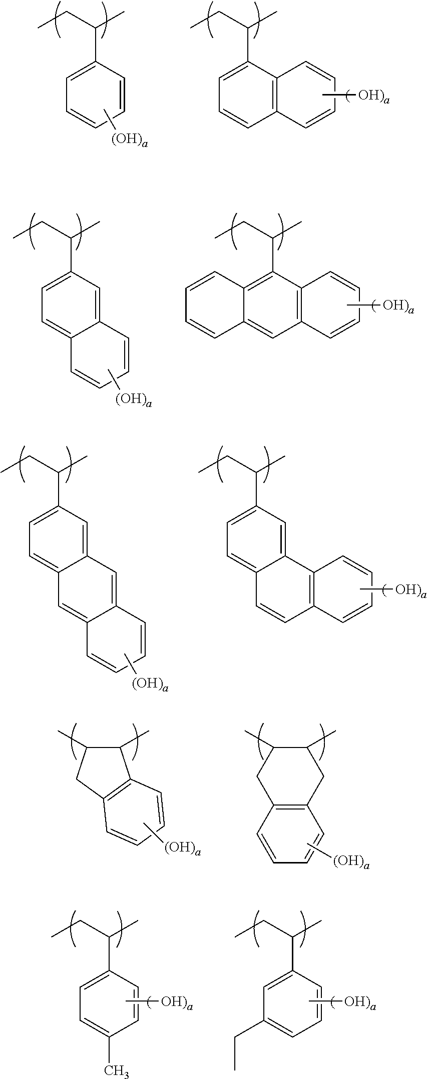

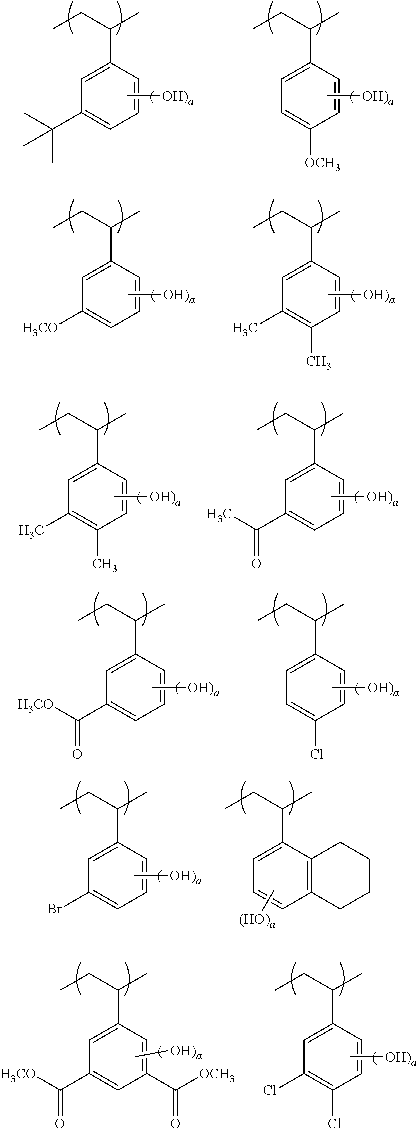

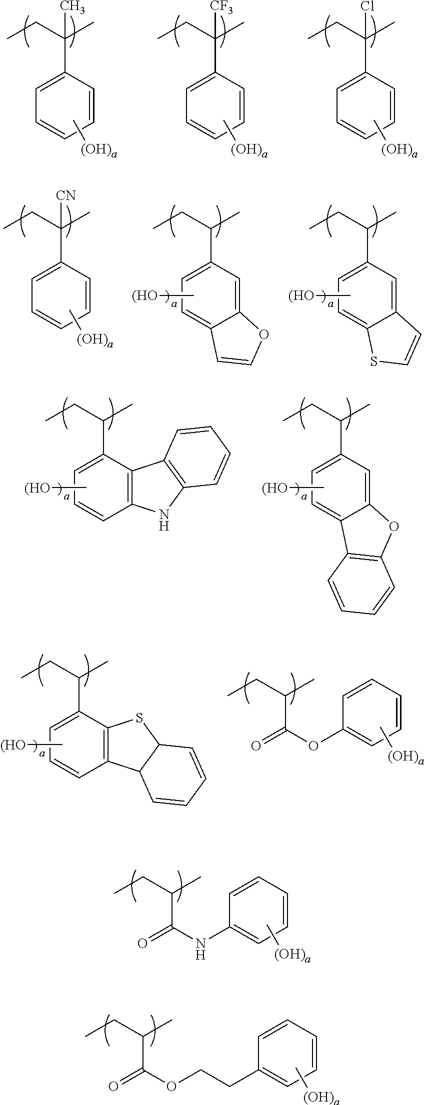

When the resin (A) includes the repeating unit represented by general formula (A2), no particular limitation is imposed on the content of the repeating unit represented by general formula (A2). The content of the repeating unit represented by general formula (A2) with respect to the total amount of the repeating units in the resin (A) is preferably 5% by mole or more, more preferably 10% by mole or more, and still more preferably 20% by mole or more. The content of the repeating unit represented by general formula (A2) with respect to the total amount of the repeating units in the resin (A) is preferably 90% by mole or less, more preferably 85% by mole or less, and still more preferably 80% by mole or less.

### <Additional Repeating Unit>

The resin (A) may include an additional repeating unit other than the above-described repeating units.

When the resin (A) includes an additional repeating unit other than the above-described repeating units, no particular limitation is imposed on the content of the additional repeating unit. The content of the additional repeating unit with respect to the total amount of the repeating units in the resin (A) is preferably 1% by mole or more and 60% by mole or less, more preferably 3% by mole or more and 50% by mole or less, and still more preferably 5% by mole or more and 40% by mole or less.

### (Repeating Unit Having Acidic Group)

The resin (A) may further have, in addition to the above-described repeating units, a repeating unit having an acidic group.

The acidic group is, for example, preferably a carboxy group, a fluorinated alcohol group (preferably a hexafluoroisopropanol group), a sulfonic acid group, a sulfonamido group, or an isopropanol group.

In the hexafluoroisopropanol group, at least one fluorine atom (preferably 1 to 2 fluorine atoms) may be substituted with a group other than a fluorine atom (for example, an alkyloxycarbonyl group). The acidic group is also preferably —C(CF₃)(OH)—CF₂— formed in the manner described above. At least one fluorine atom may be replaced with a group other than a fluorine atom to form a ring including —C(CF₃)(OH)—CF₂—.

As for specific examples of the repeating unit having the acidic group, reference can be made to, for example, the description in paragraph [0205] of WO2019/054282A, the contents of which are incorporated herein. However, the repeating unit having the acidic group is not limited thereto.

### (Repeating Unit Having Fluorine Atom or Iodine Atom and Exhibiting No Acid Decomposability)

The resin (A) may further have, in addition to the above-described repeating units, a repeating unit having a fluorine atom or an iodine atom and exhibiting no acid decomposability.

Examples of the repeating unit having a fluorine atom or an iodine atom and exhibiting no acid decomposability are shown below, but the repeating unit is not limited thereto.

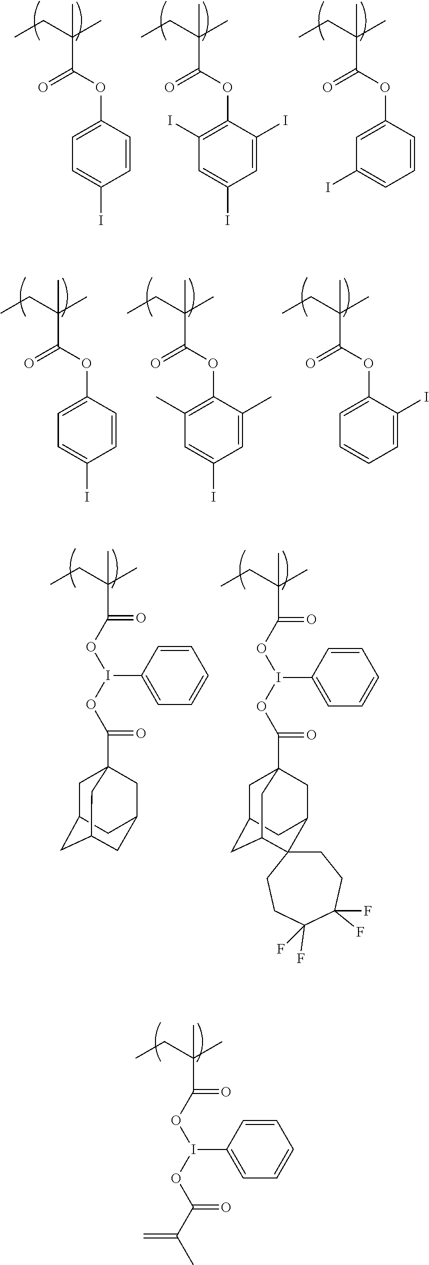

### (Repeating Unit Having Lactone Group, Sultone Group, or Carbonate Group)

The resin (A) may further have, in addition to the above-described repeating units, a repeating unit having a lactone group, a sultone group, or a carbonate group.

The lactone or sultone group may be any lactone or sultone group so long as it has a lactone or sultone structure. The lactone or sultone structure is preferably a 5- to 7-membered lactone or sultone structure. In particular, a 5- to 7-membered lactone structure with another ring structure fused thereto to form a bicyclo or spiro structure or a 5- to 7-membered sultone structure with another ring structure fused thereto to form a bicyclo or spiro structure is more preferred.

Preferably, the resin (A) has a repeating unit having a lactone or sultone group formed by removing at least one hydrogen atom from a ring member atom of a lactone structure represented by any of the following general formulas (LC1-1) to (LC1-21) or a sultone structure represented by any of the following general formulas (SL1-1) to (SL1-3).

The lactone or sultone group may be bonded directly to the main chain. For example, a ring member atom of the lactone or sultone group may be included in the main chain of the resin (A).

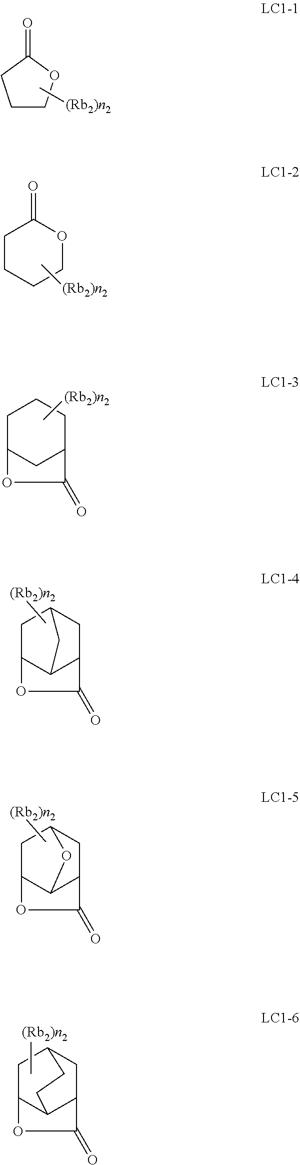

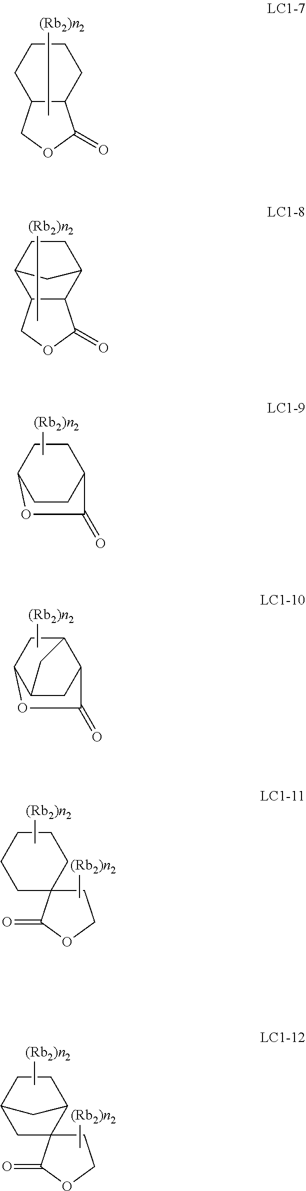

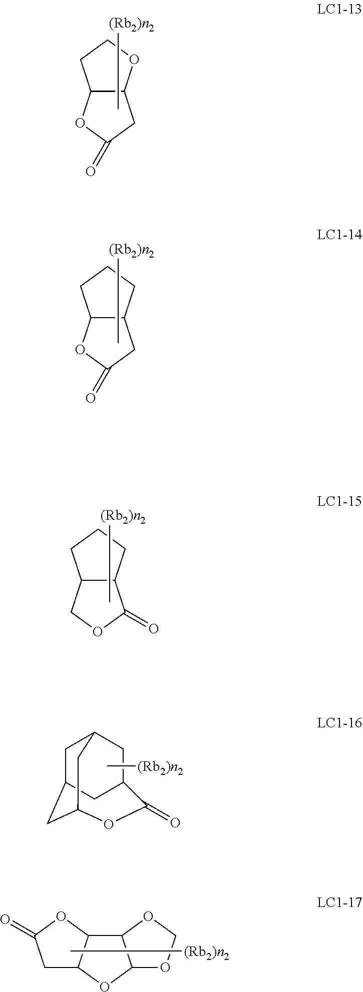

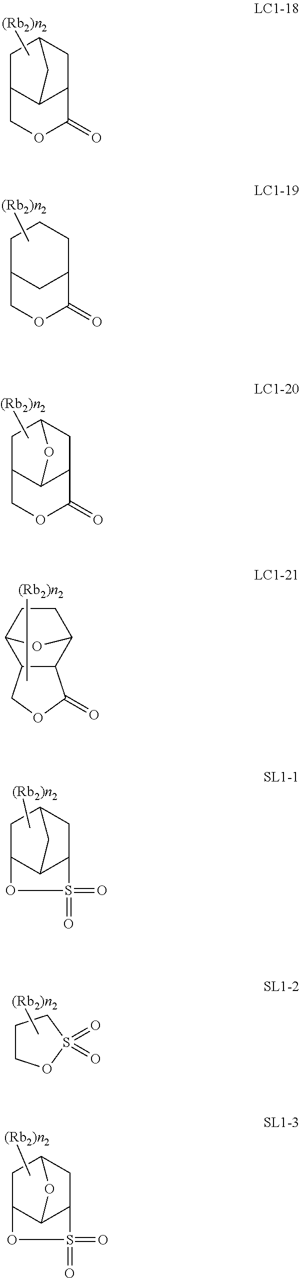

Each of the lactone and sultone structural moieties may have a substituent (Rb₂). Preferred examples of the substituent (Rb2) include alkyl groups having 1 to 8 carbon atoms, cycloalkyl groups having 4 to 7 carbon atoms, alkoxy groups having 1 to 8 carbon atoms, alkoxycarbonyl groups having 1 to 8 carbon atoms, a carboxy group, halogen atoms, a hydroxy group, a cyano group, and acid-decomposable groups. n₂ represents an integer of from 0 to 4. A plurality of Rb₂'s present when n₂ is 2 or more may be different from each other, and the plurality of Rb₂'s present may be bonded together to form a ring.

As for specific examples of the repeating unit having the lactone structure, reference can be made to, for example, the description in paragraph [0088] of WO2018/193954A, the contents of which are incorporated herein. However, the repeating unit having the lactone structure in not limited thereto.

The carbonate group is preferably a cyclic carbonate group.

### (Repeating Unit Having Photoacid Generating Group>

The resin (A) may have a repeating unit having a photoacid generating group. As for the repeating unit having a photoacid generating group, reference can be made to the description in paragraphs [0090] to [0096] of WO2018/193954A, the contents of which are incorporated herein.

### (Additional Repeating Units)

The resin (A) may have, in addition to the repeating units described above, various repeating units for the purpose of, for example, controlling dry etching resistance, suitability for a standard developer, adhesiveness to a substrate, a resist profile, resolving power, heat resistance, sensitivity, etc.

As for the additional repeating units other than the repeating units described above, reference can be made to the description in paragraphs [0097] to [0100] and [0102] to [0133] of WO2018/193954A, the contents of which are incorporated herein.

The resin (A) can be synthesized by a routine method (for example, radical polymerization).

No particular limitation is imposed on the weight average molecular weight of the resin (A). The weight average molecular weight is preferably 1000 to 200000, more preferably 2000 to 30000, and still more preferably 3000 to 20000.

The dispersity (molecular weight distribution) of the resin (A) is generally 1.0 to 5.0, preferably 1.0 to 3.0, more preferably 1.0 to 2.5, and still more preferably 1.0 to 2.0.

No particular limitation is imposed on the content of the resin (A) in the composition of the invention. The content of the resin (A) with respect to the total amount of solids in the composition of the invention is preferably 50 to 99.9% by mass, more preferably 60 to 99.0% by mass, and still more preferably 70 to 95.0% by mass.

The solids mean components other than a solvent in the composition, and any components other than the solvent are regarded as solids even when they are liquid components.

One resin (A) may be included in the composition of the invention, or two or more resins (A) may be included.

### [Acidic Compound (F) Having Iodine Atom]

The composition of the invention includes the acidic compound (F) having an iodine atom (hereinafter referred to simply as the “acidic compound (F)”).

The acidic compound (F) is a compound different from the resin (A) described above. Specifically, the acidic compound (F) and the resin (A) are different components.

The acidic compound (F) is a nonionic compound.

The pKa of the acidic compound (F) is preferably 10 or less, more preferably 7 or less, still more preferably 6 or less, particularly preferably 4 or less, and most preferably 3 or less.

The pKa of the acidic compound (F) is preferably −1 or more, more preferably 0 or more, still more preferably 1 or more, and particularly preferably 2 or more.

The number of iodine atoms included in the acidic compound (F) is preferably 1 or more and 10 or less, more preferably 1 or more and 6 or less, still more preferably 1 or more and 5 or less, and particularly preferably 1 or more and 4 or less.

The molecular weight of the acidic compound (F) is preferably 2000 or less, more preferably 1000 or less, and still more preferably 800 or less.

The Molecular Weight of the Acidic Compound (F) is Preferably 200 or More.

The acidic compound (F) is preferably a compound having an aromatic group substituted with an iodine atom. It is preferable in terms of stability that the aromatic group is substituted with an iodine atom.

When the acidic compound (F) has an aromatic group substituted with an iodine atom, the aromatic group may be an aromatic hydrocarbon group or an aromatic heterocyclic group and is preferably an aromatic hydrocarbon group.

The aromatic group may be a monocyclic aromatic group or may be a polycyclic aromatic group.

The aromatic group may have a structure formed by fusing an aromatic ring and a non-aromatic ring together.

The aromatic group may further have a substituent other than an iodine atom.

The aromatic hydrocarbon group is preferably an aromatic hydrocarbon group having 6 to 20 carbon atoms, more preferably an aromatic hydrocarbon group having 6 to 15 carbon atoms, and still more preferably an aromatic hydrocarbon group having 6 to 10 carbon atoms.

Specific examples of the aromatic hydrocarbon group include groups formed by removing at least one hydrogen atom from benzene, naphthalene, and anthracene.

The aromatic hydrocarbon group is preferably a group formed by removing at least one hydrogen atom from benzene or naphthalene and more preferably a group formed by removing at least one hydrogen atom from benzene.

The aromatic heterocyclic group is preferably an aromatic heterocyclic group including at least one heteroatom selected from the group consisting of a nitrogen atom, a sulfur atom, and an oxygen atom and more preferably an aromatic heterocyclic group including at least one nitrogen atom.

Examples of the aromatic heterocyclic group include: a group formed by removing at least one hydrogen atom from a 5-membered aromatic heterocyclic compound including at least one nitrogen atom such as pyrrole, imidazole, pyrazole, oxazole, isoxazole, thiazole, isothiazole, or triazole; and a group formed by removing at least one hydrogen atom from a 6-membered aromatic heterocyclic compound including at least one nitrogen atom such as pyridine, pyrazine, pyrimidine, pyridazine, triazine, thiazine, or oxazine.

Any carbon atom included in the aromatic heterocyclic group as a ring member may be substituted with an oxo group (═O).

Preferably, the acidic compound (F) is a compound represented by the following general formula (FA1).

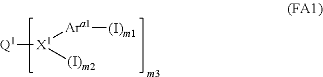

In general formula (FA1),

Ar^{a1} represents an aromatic group.

X¹ represents a single bond or a linking group.

Q¹ represents an acidic group.

Q¹ and Ar^{a1} may be bonded together to form a ring.

m1 and m2 each independently represent an integer of 0 to 5, provided that m1+m2 is 1 to 6.

When X¹ represents a single bond, m2 represents 0.

m3 represents 1 or 2.

When m3 represents 2, two Ar^{a1}'s may be the same or different, and two X¹'s may be the same or different.

Ar^{a1} in general formula (FA1) represents an aromatic group.

The description, specific examples, and preferred range of the aromatic group represented by Ar^{a1} are the same as those described above.

X¹ in general formula (FA1) represents a single bond or a linking group.

When X¹ represents a linking group, the linking group is preferably a divalent linking group. However, the divalent linking group may be substituted with m2 iodine atom(s).

When X¹ represents a divalent linking group, examples of the divalent linking group include —O—, —CO—, —COO—, —S—, —SO—, —SO₂—, —NQ²-, —NQ²CO—, hydrocarbon groups, and divalent linking groups formed by linking any of these groups. Q² represents a hydrogen atom or a substituent.

When X¹ represents a divalent linking group, the divalent linking group is preferably —O—, —CO—, —COO—, —NQ²-, —NQ²CO—, a hydrocarbon group, or a divalent linking group formed by linking any of these groups.

When X¹ represents a divalent linking group including a hydrocarbon group, the number of carbon atoms in the hydrocarbon group is preferably 1 to 20 and more preferably 1 to 10.

Examples of the hydrocarbon group include alkylene groups, cycloalkylene groups, alkenylene groups, and arylene groups.

The alkylene group may be linear or may be branched. The alkylene group may have a substituent. The alkylene group is preferably an alkylene group having 1 to 10 carbon atoms such as a methylene group, an ethylene group, a n-propylene group, an isopropylene group, or a n-butylene group and more preferably an alkylene group having 1 to 6 carbon atoms.

The cycloalkylene group may be a monocyclic cycloalkylene group or may be a polycyclic cycloalkylene group. The number of carbon atoms in the cycloalkylene group is preferably 3 to 20, more preferably 4 to 15, and still more preferably 5 to 10. The cycloalkylene group may have a substituent. Examples of the cycloalkylene group include a cyclopentylene group and a cyclohexylene group.

The arylene group may be a monocyclic arylene group or may be a polycyclic arylene group. The arylene group may have a substituent. The arylene group is preferably an arylene group having 6 to 20 carbon atoms and more preferably an arylene group having 6 to 10 carbon atoms, and examples thereof include a phenylene group and a naphthylene group.

The alkenylene group may be linear or may be branched. The alkenylene group may have a substituent. The alkenylene group is preferably an alkenylene group having 2 to 10 carbon atoms such as a vinylene group and more preferably an alkenylene group having 2 to 6 carbon atoms.

Q² represents a hydrogen atom or a substituent.

When Q² represent a substituent, no particular limitation is imposed on the substituent. The substituent is preferably an organic group having 1 to 20 carbon atoms and more preferably an organic group having 1 to 10 carbon atoms.

When Q² represents an organic group, the organic group is preferably an alkyl group, a cycloalkyl group, an alkenyl group, an aryl group, or a heterocyclic group.

When Q² represents an alkyl group, the alkyl group may be linear or may be branched. The alkyl group may have a substituent. The alkyl group is preferably an alkyl group having 1 to 10 carbon atoms such as a methyl group, an ethyl group, a n-propyl group, an isopropyl group, a n-butyl group, an isobutyl group, or t-butyl group and more preferably an alkyl group having 1 to 6 carbon atoms.

When Q² represents a cycloalkyl group, the cycloalkyl group may be a monocyclic cycloalkyl group or may be a polycyclic cycloalkyl group. The number of carbon atoms in the cycloalkyl group is preferably 3 to 20, more preferably 4 to 15, and still more preferably 5 to 10. The cycloalkyl group may have a substituent. The cycloalkyl group is preferably a monocyclic cycloalkyl group such as a cyclopentyl group or a cyclohexyl group or a polycyclic cycloalkyl group such as a norbornyl group, a tetracyclodecanyl group, a tetracyclododecanyl group, or an adamantyl group.

When Q² represents an aryl group, the aryl group may be a monocyclic aryl group or may be a polycyclic aryl group. The aryl group may have a substituent. The aryl group is preferably an aryl group having 6 to 20 carbon atoms and more preferably an aryl group having 6 to 10 carbon atoms, and examples thereof include a phenyl group, a naphthyl group, and an anthryl group.

When Q² represents an alkenyl group, the alkenyl group may be linear or may be branched. The alkenyl group may have a substituent. The alkenyl group is preferably an alkenyl group having 2 to 10 carbon atoms such as a vinyl group and more preferably an alkenyl group having 2 to 6 carbon atoms.

When Q² represents a heterocyclic group, the heterocyclic group is preferably an aromatic heterocyclic group or a non-aromatic heterocyclic group.

When Q² represents an aromatic heterocyclic group (heteroaryl group), the aromatic heterocyclic group is preferably an aromatic heterocyclic group including at least one heteroatom selected from the group consisting of a nitrogen atom, a sulfur atom, and an oxygen atom and more preferably an aromatic heterocyclic group including at least one nitrogen atom.

Examples of the aromatic heterocyclic group include: a group formed by removing at least one hydrogen atom from a 5-membered aromatic heterocyclic compound including at least one nitrogen atom such as pyrrole, imidazole, pyrazole, oxazole, isoxazole, thiazole, isothiazole, or triazole; and a group formed by removing at least one hydrogen atom from a 6-membered aromatic heterocyclic compound including at least one nitrogen atom such as pyridine, pyrazine, pyrimidine, pyridazine, triazine, thiazine, or oxazine.

The aromatic heterocyclic group may be a group formed by removing one hydrogen atom from a compound (such as indole, quinoline, or isoquinoline) obtained by fusing the above-described 5-membered aromatic heterocyclic compound or the above-described 6-membered aromatic heterocyclic compound with at least one selected from the group consisting of the above-described 5-membered aromatic heterocyclic compound, the above-described 6-membered aromatic heterocyclic compound, aromatic hydrocarbons (such as benzene and naphthalene), cycloalkanes (such as cyclopentane and cyclohexane), and non-aromatic heterocyclic compounds (such as 5-membered non-aromatic heterocyclic compounds and 6-membered non-aromatic heterocyclic compounds described later).

The aromatic heterocyclic group may have a substituent.

Any carbon atom included in the aromatic heterocyclic group as a ring member may be substituted with an oxo group (═O).

When Q² represents a non-aromatic heterocyclic group (aliphatic heterocyclic group), the non-aromatic heterocyclic group is preferably a non-aromatic heterocyclic group including at least one heteroatom selected from the group consisting of a nitrogen atom, a sulfur atom, and an oxygen atom and more preferably a non-aromatic heterocyclic group including at least one nitrogen atom.

Examples of the non-aromatic heterocyclic group include: a group formed by removing one hydrogen atom from a 5-membered non-aromatic heterocyclic compound including at least one nitrogen atom such as pyrrolidine, pyrroline, or 2-oxazolidone; and a group formed by removing one hydrogen atom from a 6-membered non-aromatic heterocyclic compound including at least one nitrogen atom such as morpholine, piperidine, or piperazine.

The non-aromatic heterocyclic group may be a group formed by removing one hydrogen atom from a group obtained by fusing the above-described 5-membered non-aromatic heterocyclic compound or the above-described 6-membered non-aromatic heterocyclic compound with at least one selected from the group consisting of the above-described 5-membered non-aromatic heterocyclic compound, the above-described 6-membered non-aromatic heterocyclic compound, and cycloalkanes (such as cyclopentane and cyclohexane).

The non-aromatic heterocyclic group may have a substituent.

Any carbon atom included in the non-aromatic heterocyclic group as a ring member may be substituted with an oxo group (═O).

Preferably, X¹ represents a single bond.

Q¹ in general formula (FA1) represents an acidic group.

No particular limitation is imposed on the acidic group, but the acidic group is preferably a phenolic hydroxy group, a carboxy group, a thiol group, a fluorinated alkyl alcohol group (such as a hexafluoroisopropanol group)), a sulfonamido group, or a sulfonamido group and more preferably a phenolic hydroxy group or a carboxy group.

Q¹ and Ar^{a1} in general formula (FA1) may be bonded together to form a ring.

m1 and m2 in general formula (FA1) each independently represent an integer of 0 to 5. However, m1+m2 is 1 to 6. When X¹ represents a single bond, m2 represents 0.

m1 represents preferably an integer of 1 to 5 and represents more preferably an integer of 1 to 4.

m2 represents preferably an integer of 0 to 3, more preferably an integer of 0 to 2, still more preferably 0 or 1, and particularly preferably 0.

m1+m2 represents preferably 1 to 4.

m3 in general formula (FA1) represents 1 or 2.

When m3 represents 2, two Ar^{a1}'s may be the same or different, and two X¹'s may be the same or different.

Preferably, m3 represents 1.

More preferably, the acidic compound (F) is a compound represented by the following general formula (FA2).

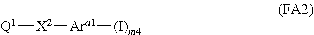

In general formula (FA2),

Ar^{a1} represents an aromatic group.

X² represents a single bond or a divalent linking group.

Q¹ represents an acidic group.

m4 represents an integer of 1 to 5.

Ar^{a1} and Q¹ in general formula (FA2) have the same meanings as Ar^{a1} and Q¹ in general formula (FA1), and their description, specific examples, and preferred range are also the same as those of Ar^{a1} and Q¹ in general formula (FA1).

X² in general formula (FA2) represents a single bond or a divalent linking group.

When X² represents a divalent linking group, the description, specific examples, and preferred range of the divalent linking group are the same as those described when X¹ represents a divalent linking group.

Preferably, X² represents a single bond.

m4 in general formula (FA2) represents an integer of 1 to 5 and represents preferably an integer of 1 to 4.

It is also preferable that the acidic compound (F) is a compound represented by the following general formula (FA3).

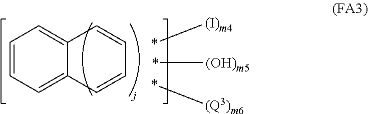

In general formula (FA3),

j represents 0 or 1.

Q³ represents a substituent.

m4 represents an integer of 1 to 5.

m5 represents an integer of 1 or more and (6+2j−m4) or less.

m6 represents an integer of 0 or more and (6+2j−m4−m5) or less.

Each * represents a direct bond bonded to an aromatic hydrocarbon shown in general formula (FA3).

j in general formula (FA3) represents 0 or 1 and represents preferably 0.

When j represents 0, the aromatic hydrocarbon shown in general formula (FA3) represents benzene.

When j represents 1, the aromatic hydrocarbon shown in general formula (FA3) represents naphthalene.

Q³ in general formula (FA3) represents a substituent.

No particular limitation is imposed on the substituent represented by Q³, but the substituent is preferably an alkyl group, a cycloalkyl group, an alkenyl group, an aryl group, a heterocyclic group, a cyano group, a nitro group, a fluorinated alkyl group, an acyl group, or an alkoxycarbonyl group.

When Q³ represents an alkyl group, a cycloalkyl group, an alkenyl group, an aryl group, or a heterocyclic group, the description, specific examples, and preferred range of the group represented by Q³ are the same as those described when Q² represents an alkyl group, a cycloalkyl group, an alkenyl group, an aryl group, or a heterocyclic group.

When Q³ represents a fluorinated alkyl group, the fluorinated alkyl group may be linear or may be branched. The fluorinated alkyl group is preferably a fluorinated alkyl group having 1 to 10 carbon atoms and more preferably a fluorinated alkyl having 1 to 6 carbon atoms. The fluorinated alkyl group may have a substituent. The fluorinated alkyl group is preferably a perfluoroalkyl group.

When Q³ represents an acyl group, the acyl group is preferably an alkylcarbonyl group and may be linear or may be branched. The alkyl group included in the alkylcarbonyl group is preferably an alkyl group having 1 to 10 carbon atoms such as a methyl group, an ethyl group, a n-propyl group, an isopropyl group, a n-butyl group, an isobutyl group, or a t-butyl group and more preferably an alkyl group having 1 to 6 carbon atoms. The acyl group may have a substituent.

When Q³ represents an alkoxycarbonyl group, the alkoxycarbonyl group may be linear or may be branched. The alkyl group included in the alkoxycarbonyl group is preferably an alkyl group having 1 to 10 carbon atoms such as a methyl group, an ethyl group, a n-propyl group, an isopropyl group, a n-butyl group, an isobutyl group, or a t-butyl group and more preferably an alkyl group having 1 to 6 carbon atoms. The alkoxycarbonyl group may have a substituent.

Q³ represents preferably a cyano group, a nitro group, a fluorinated alkyl group, an acyl group, or an alkoxycarbonyl group and represents more preferably a nitro group.

m4 in general formula (FA3) represents an integer of 1 to 5 and represents preferably an integer of 1 to 4.

m5 represents an integer of 1 or more and (6+2j−m4) or less, represents preferably 1 or 2, and represents more preferably 1.

m6 represents an integer of 0 or more and (6+2j−m4−m5) or less, represents preferably an integer of 0 to 2, and represents more preferably 1.

It is also preferable that the acidic compound (F) is a compound represented by the following general formula (FA4).

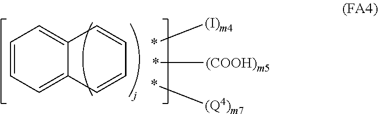

In general formula (FA4),

j represents 0 or 1.

Q⁴ represents a substituent.

m4 represents an integer of 1 to 5.

m5 represents an integer of 1 or more and (6+2j−m4) or less.

m7 represents an integer of 0 or more and (6+2j−m4−m5) or less.

Each * represents a direct bond bonded to an aromatic hydrocarbon shown in general formula (FA4).

j in general formula (FA4) represents 0 or 1 and represents preferably 0.

When j represents 0, the aromatic hydrocarbon shown in general formula (FA4) represents benzene.

When j represents 1, the aromatic hydrocarbon shown in general formula (FA4) represents naphthalene.

Q⁴ in general formula (FA4) represents a substituent.

No particular limitation is imposed on the substituent represented by Q⁴, but the substituent is preferably an alkyl group, a cycloalkyl group, an alkenyl group, an aryl group, a heterocyclic group, a hydroxy group, a cyano group, a nitro group, a fluorinated alkyl group, an acyl group, or an alkoxycarbonyl group.

When Q⁴ represents an alkyl group, a cycloalkyl group, an alkenyl group, an aryl group, or a heterocyclic group, the description, specific examples, and preferred range of the group represented by Q⁴ are the same as those described when Q² represents an alkyl group, a cycloalkyl group, an alkenyl group, an aryl group, or a heterocyclic group.

When Q⁴ represents a fluorinated alkyl group, an acyl group, or an alkoxycarbonyl group, the description, specific examples, and preferred range of the group represented by Q⁴ are the same as those described when Q³ represents a fluorinated alkyl group, an acyl group, or an alkoxycarbonyl group.

Q⁴ represents preferably an alkyl group, a cycloalkyl group, an alkenyl group, an aryl group, or a hydroxy group and represents more preferably an alkyl group or a hydroxy group.

m4 in general formula (FA4) represents an integer of 1 to 5 and represents preferably an integer of 1 to 4.

m5 represents an integer of 1 or more and (6+2j−m4) or less, represents preferably 1 or 2, and represents more preferably 1.

m7 represents an integer of 0 or more and (6+2j−m4−m5) or less, represents preferably an integer of 0 to 3, and represents more preferably an integer of 0 to 2.

It is also preferable that the acidic compound (F) is a compound represented by the following general formula (FA5).

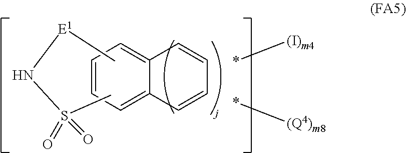

In general formula (FA5),

j represents 0 or 1.

Q⁴ represents a substituent.

E¹ represents a single bond or a divalent linking group.

m4 represents an integer of 1 to 5.

m8 represents an integer of 0 or more and (4+2j−m4) or less.

Each * represents a direct bond bonded to an aromatic hydrocarbon shown in general formula (FA5).

j in general formula (FA5) represents 0 or 1 and represents preferably 0.

When j represents 0, the aromatic hydrocarbon shown in general formula (FA5) represents benzene.

When j represents 1, the aromatic hydrocarbon shown in general formula (FA5) represents naphthalene.

Q⁴ in general formula (FA5) represents a substituent.

The description, specific examples, and preferred range of the substituent represented by Q⁴ are the same as those described for Q⁴ in general formula (FA4).

E¹ in general formula (FA5) represents a single bond or a divalent linking group and represents preferably a divalent linking group.

When E¹ represents a divalent linking group, examples of the divalent linking group include —CO—, —COO—, —S—, —SO—, —SO₂—, —NQ²-, —NQ²CO—, hydrocarbon groups, and divalent linking groups formed by linking any of these groups. Q² represents a hydrogen atom or a substituent.

The descriptions, specific examples, and preferred ranges of the hydrocarbon group and Q² are the same as those described for the hydrocarbon group and Q² in X¹ in general formula (FA1).

When E¹ represents a divalent linking group, the divalent linking group is preferably —CO— or —SO₂—.

m4 in general formula (FA5) represents an integer of 1 to 5 and represents preferably an integer of 1 to 4.

m8 represents an integer of 0 or more and (4+2j−m4) or less and represents preferably an integer of 0 to 2.

Specific examples of the acidic compound (F) are shown below, but the present invention is not limited thereto.

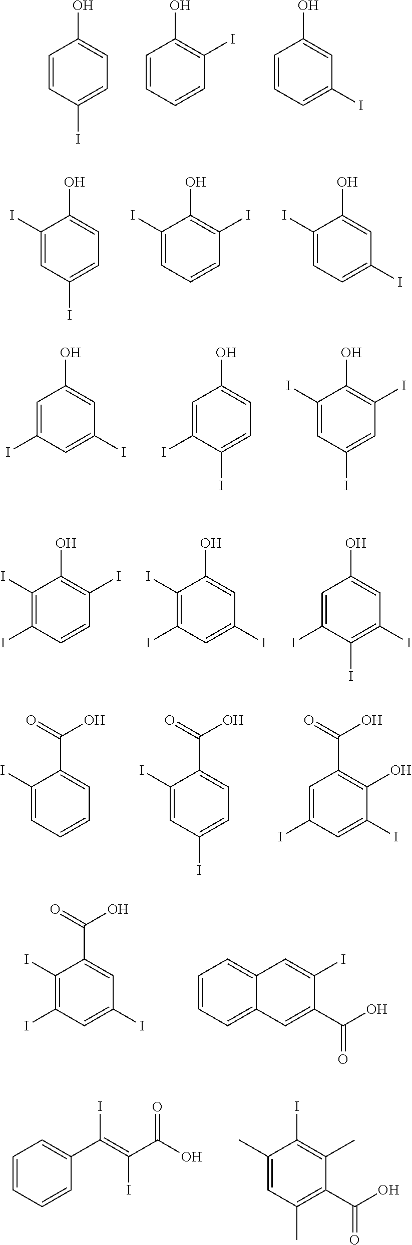

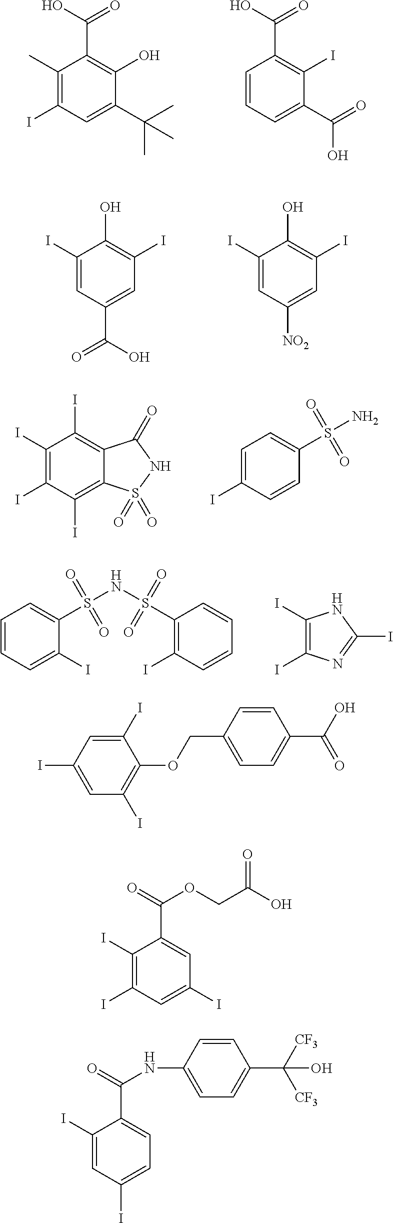

In the composition of the invention, one acidic compound (F) may be used alone, or two or more acidic compounds (F) may be used.

In the composition of the invention, the content of the acidic compound (F) (the total content when a plurality of acidic compounds (F) are present) with respect to the total amount of the solids in the composition of the invention is preferably 0.001 to 20% by mass, more preferably 0.01 to 15% by mass, and still more preferably 0.1 to 10% by mass.

The solids mean components other than a solvent in the composition, and any components other than the solvent are regarded as solids even when they are liquid components.

The acidic compound (F) may be synthesized according to a well-known method (for example, “Organic Synthesis of Bromine & Iodine compounds—Reagents and Synthetic Methods—, edited by Hitomi Suzuki, MANAC Incorporated, (2017), Maruzen Publishing,” or a commercial product may be used.

[Compound that Generates Acid Upon Irradiation with Actinic Rays or Radiation (Photoacid Generator)]

Preferably, the actinic ray-sensitive or radiation-sensitive resin composition of the invention includes a compound that generates an acid upon irradiation with actinic rays or radiation (a photoacid generator).

The compound that generates an acid upon irradiation with actinic rays or radiation is referred to also as a “compound (B)” or a “photoacid generator (B).”

The compound (B) may be in the form of a low-molecular weight compound or may be in the form in which the compound (B) is incorporated into part of a polymer (e.g., the resin (A)). A combination of the form of a low-molecular-weight compound and the form in which the compound (B) is incorporated into part of a polymer may also be used.

When the compound (B) is in the form of a low-molecular weight compound, the molecular weight of the compound (B) is preferably 3000 or less, more preferably 2000 or less, and still more preferably 1000 or less. No particular limitation is imposed on the lower limit of the molecular weight, but the molecular weight is 100 or more.

When the compound (B) is in the form in which the compound (B) is incorporated into part of a polymer, the compound (B) may be incorporated into part of the resin (A) or into a resin different from the resin (A).

In the present invention, it is preferable that the compound (B) is in the form of a low-molecular weight compound.

The compound (B) is, for example, a compound (onium salt) represented by “M⁺X⁻” and is preferably a compound that generates an organic acid upon exposure to light.

Examples of the organic acid include sulfonic acids (such as aliphatic sulfonic acids, aromatic sulfonic acids, and camphorsulfonic acid), carboxylic acids (such as aliphatic carboxylic acids, aromatic carboxylic acids, and aralkyl carboxylic acids), carbonylsulfonylimidic acid, bis(alkylsulfonyl)imidic acids, and tris(alkylsulfonyl)methide acids.

The molecular weight of the acid generated from the compound (B) is preferably 240 or more, more preferably 250 or more, still more preferably 260 or more, particularly preferably 270 or more, and most preferably 280 or more.

### <Organic Cation>

In the compound represented by “M⁺X⁻,” M⁺ represents an organic cation.

No particular limitation is imposed on the structure of the organic cation, and the valence of the organic cation may be 1 or two or more.

The organic cation is preferably a cation represented by general formula (ZaI) below (hereinafter referred to also as a “cation (ZaI)” or a cation represented by general formula (ZaII) below (hereinafter referred to also as a “cation (ZaII)”).

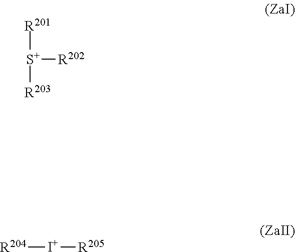

In general formula (ZaI), R²⁰¹, R²⁰², and R²⁰³ each independently represent an organic group.

In general formula (ZaII), R²⁰⁴ and R²⁰⁵ each independently represent an organic group.

General formulas (ZaI) and (ZaII) will be described later in detail. It is preferable that at least one of R²⁰¹, R²⁰², or R²⁰³ in general formula (ZaI) is an aryl group or at least one of R²⁰⁴ or R²⁰⁵ in general formula (ZaII) is an aryl group. The aryl group may have a substituent. The substituent is preferably halogen atom (preferably a fluorine atom or an iodine atom) or an organic group.

It is also preferable that at least one of R²⁰¹, R²⁰², or R²⁰³ in general formula (ZaI) has an acid-decomposable group or at least one of R²⁰⁴ or R²⁰⁵ in general formula (ZaII) has an acid-decomposable group. The acid-decomposable group is the same as the acid-decomposable group in the resin (A).

The mode in which at least one of R²⁰¹, R²⁰², or R²⁰³ in general formula (ZaI) has an acid-decomposable group is preferably a mode in which at least one of R²⁰¹, R²⁰², or R²⁰³ is an aryl group substituted with an organic group including the acid-decomposable group. The mode in which at least one of R²⁰⁴ or R²⁰⁵ in general formula (ZaII) has an acid-decomposable group is preferably a mode in which at least one of R²⁰⁴ or R²⁰⁵ is an aryl group substituted with an organic group including the acid-decomposable group.

The cation (ZaI) will be described.

The number of carbon atoms in each of the organic groups represented by R²⁰¹, R²⁰², and R²⁰³ is generally 1 to 30 and preferably 1 to 20. Two selected from the group consisting of R²⁰¹ to R²⁰³ may be bonded together to form a ring structure, and the ring may include an oxygen atom, a sulfur atom, an ester group, an amido group, or a carbonyl group. Examples of the group formed from two selected from the group consisting of R²⁰¹ to R²⁰³ that are bonded together include alkylene groups (such as a butylene group and a pentylene group) and —CH₂—CH₂—O—CH₂—CH₂—.

Preferred examples of the form of the organic cation in formula (ZaI) include a cation (ZaI-1), a cation (ZaI-2), an organic cation (cation (ZaI-3b)) represented by formula (ZaI-3b), and an organic cation (cation (ZaI-4b)) represented by formula (ZaI-4b) that will be described later.

First, the cation (ZaI-1) will be described.

The cation (ZaI-1) is an arylsulfonium cation in which at least one of R²⁰¹, R²⁰², or R²⁰³ in formula (ZaI) is an aryl group.

In the arylsulfonium cation, each of R²⁰¹ to R²⁰³ may be an aryl group. Alternatively, some of R²⁰¹ to R²⁰³ may be an aryl group, and the rest may be an alkyl group or a cycloalkyl group.

Alternatively, one of R²⁰¹, R²⁰², or R²⁰³ may be an aryl group, and the remaining two of R²⁰¹ to R²⁰³ may be bonded together to form a ring structure. The ring may include an oxygen atom, a sulfur atom, an ester group, an amido group, or a carbonyl group. Examples of the group formed by bonding two selected from the group consisting of R²⁰¹ to R²⁰³ together include alkylene groups in which at least one methylene group is replaced by an oxygen atom, a sulfur atom, an ester group, an amido group, and/or a carbonyl group (such as a butylene group, a pentylene group, and a —CH₂—CH₂—O—CH₂—CH₂—).

Examples of the arylsulfonium cation include a triarylsulfonium cation, diarylalkylsulfonium cations, aryldialkylsulfonium cations, diarylcycloalkylsulfonium cations, and aryldicycloalkylsulfonium cations.

Each aryl group included in the arylsulfonium cation is preferably a phenyl group or a naphthyl group and is more preferably a phenyl group. The aryl group may have a heterocyclic structure having an oxygen atom, a nitrogen atom, a sulfur atom, etc. Examples of the heterocyclic structure include a pyrrole residue, a furan residue, a thiophene residue, an indole residue, a benzofuran residue, and a benzothiophene residue. When the arylsulfonium cation has two or more aryl groups, the two or more aryl groups may be the same or different.

The alkyl group or the cycloalkyl group optionally included in the arylsulfonium cation is preferably a linear alkyl group having 1 to 15 carbon atoms, a branched alkyl group having 3 to 15 carbon atoms, or a cycloalkyl group having 3 to 15 carbon atoms and more preferably a methyl group, an ethyl group, a propyl group, a n-butyl group, a sec-butyl group, a t-butyl group, a cyclopropyl group, a cyclobutyl group, or a cyclohexyl group.

The aryl, alkyl, and cycloalkyl groups in R²⁰¹ to R²⁰³ may each independently have a substituent, and the substituent is preferably an alkyl group (having, for example, 1 to 15 carbon atoms), a cycloalkyl group (having, for example, 3 to 15 carbon atoms), an aryl group (having, for example, 6 to 14 carbon atoms), an alkoxy group (having, for example, 1 to 15 carbon atoms), a cycloalkylalkoxy group (having, for example, 1 to 15 carbon atoms), a halogen atom (for example, fluorine or iodine), a hydroxy group, a carboxy group, an ester group, a sulfinyl group, a sulfonyl group, an alkylthio group, or a phenylthio group.

Each substituent may have a substituent if possible. It is also preferable that the alkyl group has a halogen atom as a substituent and is therefore a halogenated alkyl group such as a trifluoromethyl group.

It is also preferable that any of these substituents are combined together to form an acid-decomposable group.

The acid-decomposable group means a group that is decomposed by the action of an acid to generate a polar group and preferably has a structure in which the polar group is protected by a leaving group that leaves by the action of an acid. The polar group and the leaving group are as described above.

Next, the cation (ZaI-2) will be described.

The cation (ZaI-2) is a cation in which R²⁰¹ to R²⁰³ in formula (ZaI) each independently represent an organic group having no aromatic ring. The aromatic ring is intended to encompass an aromatic ring including a heteroatom.

The number of carbon atoms in each of the organic groups having no aromatic ring and represented by R²⁰¹ to R²⁰³ is generally 1 to 30 and preferably 1 to 20.

R²⁰¹ to R²⁰³ each independently represent preferably an alkyl group, a cycloalkyl group, an allyl group, or a vinyl group, more preferably a linear or branched 2-oxoalkyl group, a 2-oxocycloalkyl group, or an alkoxycarbonylmethyl group, and still more preferably a linear or branched 2-oxoalkyl group.

Examples of the alkyl and cycloalkyl groups in R²⁰¹ to R²⁰³ include: linear alkyl groups having 1 to 10 carbon atoms and branched alkyl groups having 3 to 10 carbon atoms (such as a methyl group, an ethyl group, a propyl group, a butyl group, and a pentyl group); and cycloalkyl groups having 3 to 10 carbon atoms (such as a cyclopentyl group, a cyclohexyl group, and a norbornyl group).

R²⁰¹ to R²⁰³ may each be further substituted with a halogen atom, an alkoxy group (having, for example, 1 to 5 carbon atoms), a hydroxy group, a cyano group, or a nitro group.

It is also preferable that the substituents in R²⁰¹ to R²⁰³ are each independently combined with another substituent to form an acid-decomposable group.

Next, the cation (ZaI-3b) will be described.

The cation (ZaI-3b) is a cation represented by the following formula (ZaI-3b).

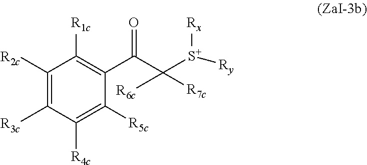

In formula (ZaI-3b),

R_{1c} to R_{5c} each independently represent a hydrogen atom, an alkyl group, a cycloalkyl group, an aryl group, an alkoxy group, an aryloxy group, an alkoxycarbonyl group, an alkylcarbonyloxy group, a cycloalkylcarbonyloxy group, a halogen atom, a hydroxy group, a nitro group, an alkylthio group, or an arylthio group.

R_{6c} and R_{7c} each independently represent a hydrogen atom, an alkyl group (such as a t-butyl group), a cycloalkyl group, a halogen atom, a cyano group, or an aryl group.

Rₓ and R_{y} each independently represent an alkyl group, a cycloalkyl group, a 2-oxoalkyl group, a 2-oxocycloalkyl group, an alkoxycarbonylalkyl group, an allyl group, or a vinyl group.

R_{1c} to R_{7c}, Rₓ, and R_{y} may each have a substituent, and it is also preferable that these substituents are each independently combined with another substituent to form an acid-decomposable group.

A combination of two or more selected from the group consisting of R_{1c} to R_{5c}, a pair of R_{5c} and R_{6c}, a pair of R_{6c} and R_{7c}, a pair of R_{5c} and Rₓ, and a pair of Rₓ and R_{y} may each be bonded together to form a ring. These rings may each independently include an oxygen atom, a sulfur atom, a ketone group, an ester bond, or an amide bond.

Each ring may be an aromatic or non-aromatic hydrocarbon ring, an aromatic or non-aromatic heterocyclic ring, or a polycyclic condensed ring formed by combining two or more of the above rings. The ring may be a 3- to 10-membered ring and is preferably a 4- to 8-membered ring and more preferably a 5- or 6-membered ring.

Examples of the groups formed by bonding two or more selected from the group consisting of R_{1c} to R_{5c}, bonding R_{6c} and R_{7c}, and bonding Rₓ and R_{y} include alkylene groups such as a butylene group and a pentylene group. A methylene group in the alkylene group may be replaced with a heteroatom such as an oxygen atom.

The group formed by bonding R_{5c} and R_{6c} and the group formed by bonding R_{5c} and Rₓ are each preferably a single bond or an alkylene group. Examples of the alkylene group include a methylene group and an ethylene group.

R_{1c} to R_{5c}, R_{6c}, R_{7c}, Rₓ, R_{y}, the ring formed by bonding together a combination of two or more selected from the group consisting of R_{1c} to R_{5c}, the ring formed by bonding together a pair of R_{5c} and R_{6c}, the ring formed by bonding together a pair of R_{6c} and R_{7c}, the ring formed by bonding together a pair of R_{5c} and Rₓ, and the ring formed by bonding together a pair of Rₓ and R_{y} may each have a substituent.

Next, the cation (ZaI-4b) will be described.

The cation (ZaI-4b) is a cation represented by the following formula (ZaI-4b).

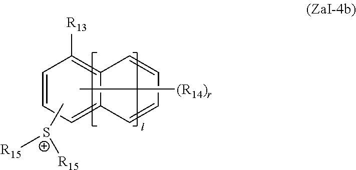

In formula (ZaI-4b),

l represents an integer of from 0 to 2.

r represents an integer of from 0 to 8.

R₁₃ represents a hydrogen atom, a halogen atom (such as a fluorine atom or an iodine atom), a hydroxy group, an alkyl group, a halogenated alkyl group, an alkoxy group, a carboxy group, an alkoxycarbonyl group, or a group including a cycloalkyl group (a cycloalkyl group itself or a group including a cycloalkyl group as a part thereof). These groups may each have a substituent.

R₁₄ represents a hydroxy group, a halogen atom (such as a fluorine atom or an iodine atom), an alkyl group, a halogenated alkyl group, an alkoxy group, an alkoxycarbonyl group, an alkylcarbonyl group, an alkylsulfonyl group, a cycloalkylsulfonyl group, or a group including a cycloalkyl group (a cycloalkyl group itself or a group including a cycloalkyl group as a part thereof). These groups may each have a substituent. When a plurality of R₁₄'s are present, they each independently represent any of the above groups such as a hydroxy group.

R₁₅'s each independently represent an alkyl group, a cycloalkyl group, or a naphthyl group. The two R₁₅'s may be bonded together to form a ring. When the two R₁₅'s are bonded together to form a ring, the skeleton of the ring may include a heteroatom such as an oxygen atom or a nitrogen atom. In one preferred mode, the two R₁₅'s are each an alkylene group and are bonded together to form a ring structure. The above alkyl, cycloalkyl, and naphthyl groups and the ring formed by bonding the two R₁₅'s may each have a substituent.

In formula (ZaI-4b), the alkyl group represented by each of R₁₃, R₁₄, and R₁₅s may be a linear or branched alkyl group. Preferably, the number of carbon atoms in the alkyl group is 1 to 10. Each alkyl group is more preferably a methyl group, an ethyl group, a n-butyl group, a t-butyl group, etc.

It is also preferable that the substituents in R₁₃ to R₁₅'s are each independently combined with another substituent to form an acid-decomposable group.

Next, formula (ZaII) will be described.

In formula (ZaII), R²⁰⁴ and R²⁰⁵ each independently represent an organic group and represent preferably an aryl group, an alkyl group, or a cycloalkyl group.

The aryl group represented by each of R²⁰⁴ and R²⁰⁵ is preferably a phenyl group or a naphthyl group and more preferably a phenyl group. The aryl group represented by each of R²⁰⁴ and R²⁰⁵ may be an aryl group having a heterocycle having an oxygen atom, a nitrogen atom, or a sulfur atom. Examples of the skeleton of the aryl group having a heterocycle include pyrrole, furan, thiophene, indole, benzofuran, and benzothiophene.

The alkyl or cycloalkyl group represented by each of R²⁰⁴ and R²⁰⁵ is preferably a linear alkyl group having 1 to 10 carbon atoms or a branched alkyl group having 3 to 10 carbon atoms (such as a methyl group, an ethyl group, a propyl group, a butyl group, or a pentyl group) or is preferably a cycloalkyl group having 3 to 10 carbon atoms (such as a cyclopentyl group, a cyclohexyl group, or a norbornyl group).

The aryl, alkyl, and cycloalkyl groups represented by R²⁰4 and R²⁰⁵ may each independently have a substituent. Examples of the optional substituents in the aryl, alkyl, and cycloalkyl groups represented by R²⁰⁴ and R²⁰⁵ include alkyl groups (having, for example, 1 to 15 carbon atoms), cycloalkyl groups (having, for example, 3 to 15 carbon atoms), aryl groups (having, for example, 6 to 15 carbon atoms), alkoxy groups (having, for example, 1 to 15 carbon atoms), halogen atoms, a hydroxy group, and a phenylthio group. It is also preferable that the substituents in R²⁰⁴ and R²⁰⁵ are each independently combined with another substituent to form an acid-decomposable group.

Specific examples of the organic cation represented by M⁺ are shown below. However, the invention is not limited thereto.

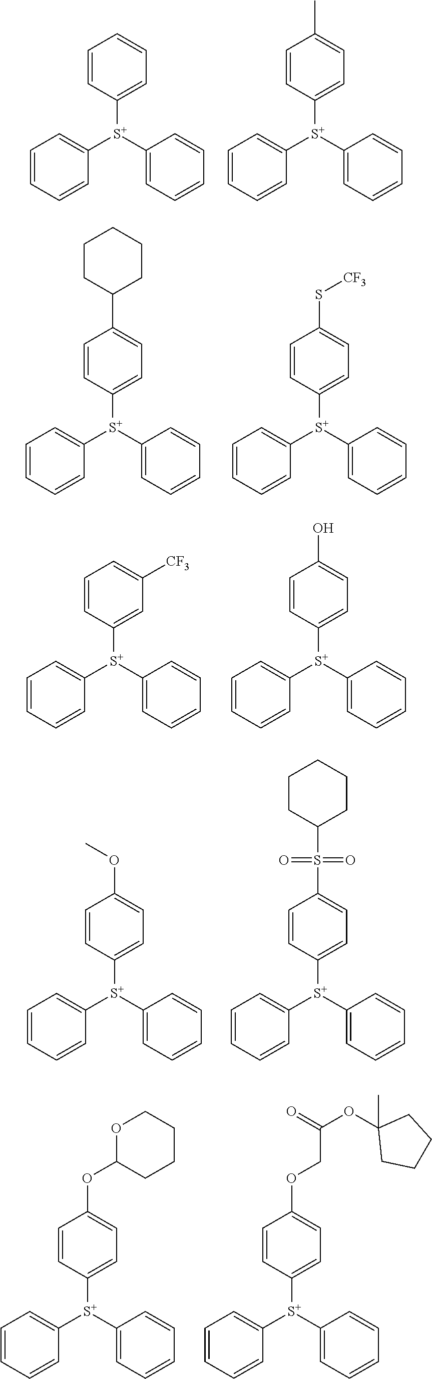

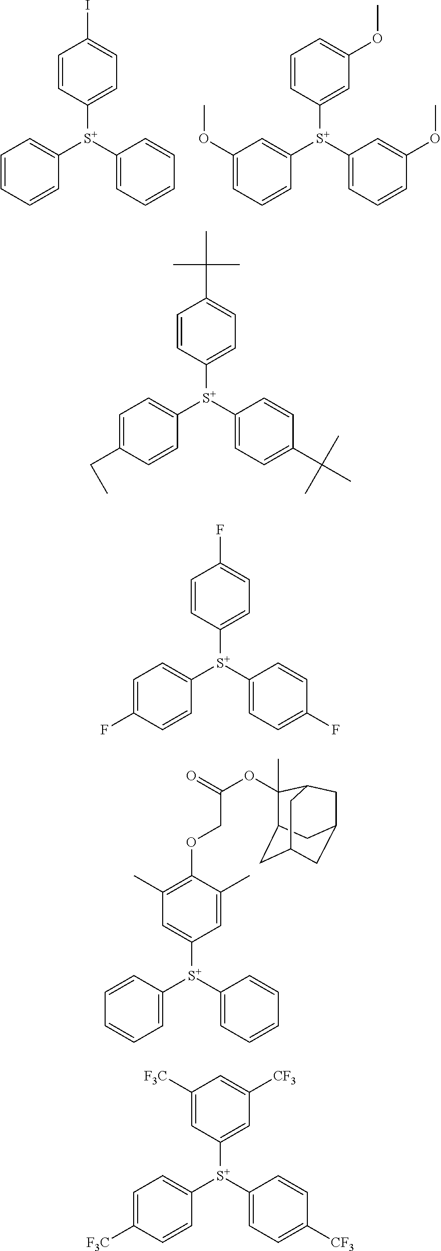 [SMILES: Ic1ccc([S+](c2ccccc2)c2ccccc2)cc1 ; COc1cccc([S+](c2cccc(OC)c2)c2cccc(OC)c2)c1 ; CCc1ccc([S+](c2ccc(C(C)(C)C)cc2)c2ccc(C(C)(C)C)cc2)cc1 ; Fc1ccc([S+](c2ccc(F)cc2)c2ccc(F)cc2)cc1 ; Cc1cc([S+](c2ccccc2)c2ccccc2)cc(C)c1OCC(=O)OC1(C)C2CC3CC(C2)CC1C3 ; FC(F)(F)c1ccc([S+](c2ccc(C(F)(F)F)cc2)c2cc(C(F)(F)F)cc(C(F)(F)F)c2)cc1]

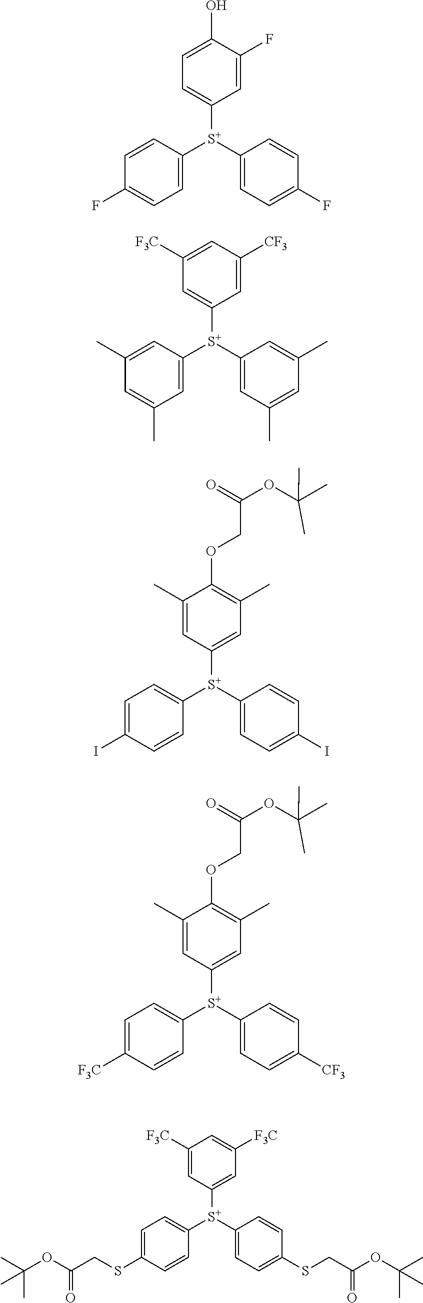

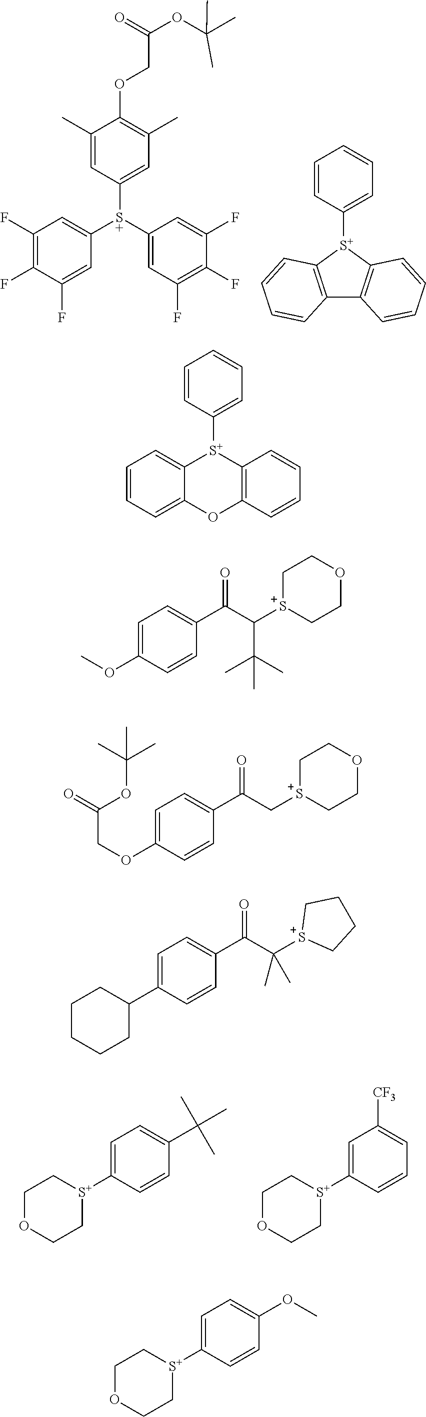

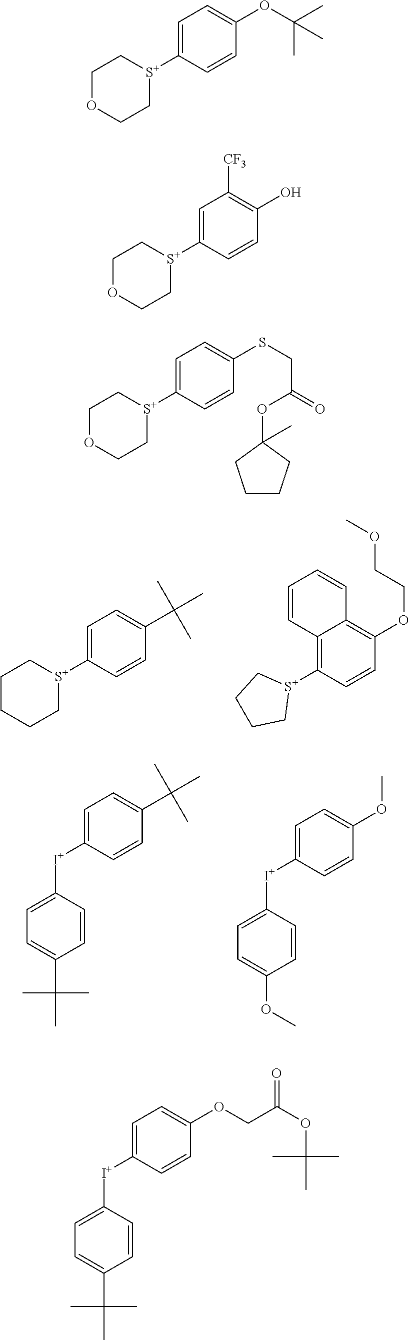

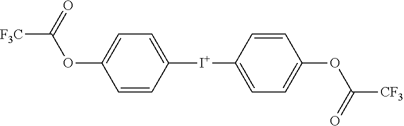 [SMILES: O=C(Oc1ccc([I+]c2ccc(OC(=O)C(F)(F)F)cc2)cc1)C(F)(F)F]

### <Organic Anion>

In the compound represented by “M⁺X⁻,” X⁻ represents an organic anion.

No particular limitation is imposed on the organic anion, and examples thereof include monovalent organic anions and divalent and higher valent organic anions.

The organic anion is preferably an anion whose ability to cause a nucleophilic reaction is very low and is more preferably a non-nucleophilic anion.

Examples of the non-nucleophilic anion include sulfonate anions (such as aliphatic sulfonate anions, aromatic sulfonate anions, and a camphorsulfonate anion), carboxylate anions (such as aliphatic carboxylate anions, aromatic carboxylate anions, and aralkyl carboxylate anions), sulfonylimide anions, bis(alkylsulfonyl)imide anions, and tris(alkylsulfonyl)methide anions.

In the aliphatic sulfonate anions and the aliphatic carboxylate anions, the aliphatic moiety may be a linear or branched alkyl group or a cycloalkyl group and is preferably a linear or branched alkyl group having 1 to 30 carbon atoms or a cycloalkyl group having 3 to 30 carbon atoms.

The alkyl group may be, for example, a fluoroalkyl group (which may have a substituent other than a fluorine atom or may be a perfluoroalkyl group).

In the aromatic sulfonate anions and the aromatic carboxylate anions, the aryl group is preferably an aryl group having 6 to 14 carbon atoms such as a phenyl group, a tolyl group, or a naphthyl group.

The above-described alkyl, cycloalkyl, and aryl groups may each have a substituent. No particular limitation is imposed on the substituent, and specific examples thereof include a nitro group, halogen atoms such as a fluorine atom and a chlorine atom, a carboxy group, a hydroxy group, an amino group, a cyano group, alkoxy groups (having preferably 1 to 15 carbon atoms), alkyl groups (having preferably 1 to 10 carbon atoms), cycloalkyl groups (having preferably 3 to 15 carbon atoms), aryl groups (having preferably 6 to 14 carbon atoms), alkoxycarbonyl groups (having preferably 2 to 7 carbon atoms), acyl groups (having preferably 2 to 12 carbon atoms), alkoxycarbonyloxy groups (having preferably 2 to 7 carbon atoms), alkylthio groups (having preferably 1 to 15 carbon atoms), alkylsulfonyl groups (having preferably 1 to 15 carbon atoms), alkyliminosulfonyl groups (having preferably 1 to 15 carbon atoms), and aryloxysulfonyl groups (having preferably 6 to 20 carbon atoms).

In the aralkyl carboxylate anions, the aralkyl group is preferably an aralkyl group having 7 to 14 carbon atoms. Examples of the aralkyl group having 7 to 14 carbon atoms include a benzyl group, a phenethyl group, a naphthylmethyl group, a naphthylethyl group, and a naphthylbutyl group.

Examples of the sulfonylimide anion include a saccharin anion.

In the bis(alkylsulfonyl)imide anions and the tris(alkylsulfonyl)methide anions, the alkyl group is preferably an alkyl group having 1 to 5 carbon atoms. These alkyl groups may have a substituent, and examples of the substituent include halogen atoms, alkyl groups substituted with halogen atoms, alkoxy groups, alkylthio groups, alkyloxysulfonyl groups, aryloxysulfonyl groups, and cycloalkylaryloxysulfonyl groups. The substituent is preferably a fluorine atom or an alkyl group substituted with a fluorine atom.

In the bis(alkylsulfonyl)imide anions, the alkyl groups may be bonded together to form a ring structure. In this case, the strength of the acid increases.

Other examples of the non-nucleophilic anion include phosphorus fluoride (such as PF₆), boron fluoride (such as BF₄⁻), and antimony fluoride (such as SbF₆⁻).

The non-nucleophilic anion is preferably an aliphatic sulfonate anion substituted with a fluorine atom at least at the α-position of the sulfonic acid, an aromatic sulfonate anion substituted with a fluorine atom or a fluorine atom-containing group, a bis(alkylsulfonyl)imide anion in which an alkyl group is substituted with a fluorine atom, or a tris(alkylsulfonyl)methide anion in which an alkyl group is substituted with a fluorine atom. In particular, the non-nucleophilic anion is more preferably a perfluoroaliphatic sulfonate anion (having preferably 4 to 8 carbon atoms) or a benzenesulfonate anion having a fluorine atom and still more preferably a nonafluorobutanesulfonate anion, a perfluorooctanesulfonate anion, a pentafluorobenzenesulfonate anion, or a 3,5-bis(trifluoromethyl)benzenesulfonate anion.

Preferred examples of the non-nucleophilic anion include an anion represented by the following formula (AN4).

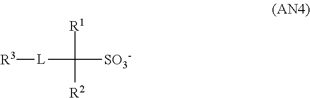

In formula (AN4), R¹ to R³ each independently represent an organic group or a hydrogen atom. L represents a divalent linking group.

In formula (AN4), L represents a divalent linking group.

When a plurality of L's are present, L's may be the same or different.

Examples of the divalent linking group include —O—CO—O—, —COO—, —CONH—, —CO—, —O—, —S—, —SO—, —SO₂—, alkylene groups (having preferably 1 to 6 carbon atoms), cycloalkylene groups (having preferably 3 to 15 carbon atoms), alkenylene groups (having preferably 2 to 6 carbon atoms), and divalent linking groups formed by combining any of the above groups. In particular, the divalent linking group is preferably —O—CO—O—, —COO—, —CONH—, —CO—, —O—, —SO₂—, —O—CO—O-alkylene group-, —COO-alkylene group-, or —CONH-alkylene group- and more preferably —O—CO—O—, —O—CO—O-alkylene group-, —COO—, —CONH—, —SO₂—, or —COO-alkylene group-.

For example, L is preferably a group represented by the following formula (AN4-2).

*^{a}—(CR^{2a}₂)_{X}-Q-(CR^{2b}₂)_{Y}—*^{b} (AN4-2)

In formula (AN4-2), *^{a} represents a bonding position to R³ in formula (AN4).

*^{b} represents a bonding position to —C(R¹)(R²)— in formula (AN4).

X and Y each independently represent an integer of from 0 to 10 and preferably an integer of 0 to 3.

R^{2a} and R^{2b} each independently represent a hydrogen atom or a substituent.

When a plurality of R^{2a}'s are present, they may be the same or different. When a plurality of R^{2b}'s are present, they may be the same or different.

When Y is 1 or more, R^{2b} in CR^{2b}₂ that is bonded directly to —C(R¹)(R²)— in formula (AN4) differs from a fluorine atom.

Q represents *^{A}—O—CO—O—*^{B}, *^{A}—CO—*^{B}, *^{A}—CO—O—*^{B}, *^{A}—O—CO—*^{B}, *^{A}—O—*^{B}—*^{A}—S—*^{B}, or *^{A}—SO₂—*^{B},

When X+Y in formula (AN4-2) is 1 or more and R^{2a}'s and R^{2b}'s in formula (AN4-2) are each a hydrogen atom, Q represents *^{A}—O—CO—O—*^{B}, *^{A}—CO—*^{B}, *^{A}—O—CO—*^{B}, *^{A}—O—*^{B}, *^{A}—S—*^{B}, or *A-SO₂—*^{B}.

*^{A} represents a bonding position on the R³ side in formula (AN4), and *^{B} represents a bonding position on the —SO₃— side in formula (AN4).

In formula (AN4), R¹ to R³ each independently represent an organic group.

No particular limitation is imposed on the organic group, so long as it has at least one carbon atom. The organic group may be a linear group (e.g., a linear alkyl group), a branched group (e.g., a branched alkyl group such as a t-butyl group), or a cyclic group. The organic group may or may not have a substituent. The organic group may or may not have a heteroatom (such as an oxygen atom, a sulfur atom, and/or a nitrogen atom).

Other examples of the organic group include substituent groups other than electron-withdrawing groups.

Examples of the substituent group other than electron-withdrawing groups include hydrocarbon groups, a hydroxy group, oxyhydrocarbon groups, oxycarbonyl hydrocarbon groups, an amino group, hydrocarbon-substituted amino groups, and hydrocarbon-substituted amido groups.

Preferably, these substituent groups other than electron-withdrawing groups are each independently —R′, —OH, —OR′, —OCOR′, —NH₂, —NR′₂, —NHR′, or —NHCOR′, R′ is a monovalent hydrocarbon group.

Examples of the monovalent hydrocarbon group represented by R′ include: linear or branched monovalent hydrocarbon groups including alkyl groups such as a methyl group, an ethyl group, a propyl group, and a butyl group, alkenyl groups such as an ethenyl group, a propenyl group, and a butenyl group, and alkynyl groups such as an ethynyl group, a propynyl group, and a butynyl group; monovalent alicyclic hydrocarbon groups including cycloalkyl groups such as a cyclopropyl group, a cyclobutyl group, a cyclopentyl group, a cyclohexyl group, a norbornyl group, and a adamantyl group and cycloalkenyl groups such as a cyclopropenyl group, a cyclobutenyl group, a cyclopentenyl group, and a norbornenyl group; and monovalent aromatic hydrocarbon groups including aryl groups such as a phenyl group, a tolyl group, a xylyl group, a mesityl group, a naphthyl group, a methylnaphthyl group, an anthryl group, and a methylanthryl group and aralkyl groups such as a benzyl group, a phenethyl group, a phenylpropyl group, a naphthylmethyl group, and an anthrylmethyl group.

In particular, it is preferable that R¹ and R² each independently represent a hydrocarbon group (preferably a cycloalkyl group) or a hydrogen atom.

In particular, R³ is preferably an organic group having a ring structure. The ring structure may be a monocyclic structure or a polycyclic structure and may have a substituent. Preferably, the ring in the organic group including the ring structure is bonded directly to L in formula (AN4).

The organic group having the ring structure may or may not have, for example, a heteroatom (for example, an oxygen atom, a sulfur atom, and/or, a nitrogen atom). At least one carbon atom included in the ring structure may be replaced with a heteroatom.

The organic group having the ring structure is preferably a hydrocarbon group having a ring structure, a lactone ring group, or a sultone ring group. In particular, the organic group having the ring structure is preferably a hydrocarbon group having a ring structure.

The hydrocarbon group having a ring structure is preferably a monocyclic or polycyclic cycloalkyl group. These groups may have a substituent.

The cycloalkyl group may be a monocyclic group (such as a cyclohexyl group) or a polycyclic group (such as an adamantyl group), and the number of carbon atoms is preferably 5 to 12.

Preferably, the lactone group and the sultone group are each, for example, a group formed by removing one hydrogen atom from a ring member atom included in the lactone or sultone structure in any of the structures represented by formulas (LC1-1) to (LC1-21) and formulas (SL1-1) to (SL1-3) described above.

The non-nucleophilic anion is also preferably an anion represented by the following formula (AN1).

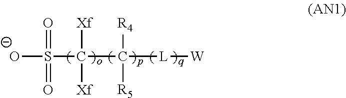

In formula (AN1), o represents an integer of from 1 to 3. p represents an integer of from 0 to 10. q represents an integer of from 0 to 10.

Xf represents a fluorine atom or an organic group. The organic group may be an organic group substituted with at least one fluorine atom or an organic group having no fluorine atom. The number of carbon atoms in the organic group (preferably an alkyl group) is preferably 1 to 10 and more preferably 1 to 4. The organic group (preferably an alkyl group) substituted with at least one fluorine atom is preferably a perfluoroalkyl group.

At least one Xf is preferably a fluorine atom or a perfluoroalkyl group having 1 to 4 carbon atoms and more preferably a fluorine atom or CF₃. It is still more preferable that each of Xf's is a fluorine atom.

R₄ and R₅ each independently represent a hydrogen atom, a fluorine atom, an alkyl group, or an alkyl group substituted with at least one fluorine atom. When a plurality of R₄'s are present, they may be the same or different. When a plurality of R₅'s are present, they may be the same or different.

The number of carbon atoms in each of the alkyl groups represented by R₄ and R₅ is preferably 1 to 4. Each alkyl group may have a substituent. R₄ and R₅ are each preferably a hydrogen atom.

Specific examples and preferred modes of the alkyl group substituted with at least one fluorine atom are the same as the specific examples and preferred modes of Xf in formula (AN1).

L represents a divalent linking group.

When a plurality of L's are present, they may be the same or different.

Examples of the divalent linking group include —O—CO—O—, —COO—, —CONH—, —CO—, —O—, —S—, —SO—, —SO₂—, alkylene groups (having preferably 1 to 6 carbon atoms), cycloalkylene groups (having preferably 3 to 15 carbon atoms), alkenylene groups (having preferably 2 to 6 carbon atoms), and divalent linking groups formed by combining any of these groups. In particular, the divalent linking group is preferably —O—CO—O—, —COO—, —CONH—, —CO—, —O—, —SO₂—, —O—CO—O-alkylene group-, —COO-alkylene group-, or —CONH-alkylene group- and more preferably —O—CO—O—, —O—CO—O-alkylene group-, —COO—, —CONH—, —SO₂—, or —COO-alkylene group-.

W represents an organic group including a ring structure. In particular, W is preferably a cyclic organic group.

Examples of the cyclic organic group include alicyclic groups, aryl groups, and heterocyclic groups.

The alicyclic group may be a monocyclic alicyclic group or a polycyclic alicyclic group. Examples of the monocyclic alicyclic group include monocyclic cycloalkyl groups such as a cyclopentyl group, a cyclohexyl group, and a cyclooctyl group. Examples of the polycyclic alicyclic group include polycyclic cycloalkyl groups such as a norbornyl group, a tricyclodecanyl group, a tetracyclodecanyl group, a tetracyclododecanyl group, and an adamantyl group. Of these, alicyclic groups having 7 or more carbon atoms and a bulky structure such as a norbornyl group, a tricyclodecanyl group, a tetracyclodecanyl group, a tetracyclododecanyl group, and an adamantyl group are preferred.

The aryl group may be a monocyclic or polycyclic aryl group. Examples of such an aryl group include a phenyl group, a naphthyl group, a phenanthryl group, and an anthryl group.

The heterocyclic group may be a monocyclic or polycyclic heterocyclic group. In particular, when the heterocyclic group is a polycyclic heterocyclic group, diffusion of acid can be further reduced. The heterocyclic group may or may not have aromaticity. Examples of the heterocycle having aromaticity include a furan ring, a thiophene ring, a benzofuran ring, a benzothiophene ring, a dibenzofuran ring, a dibenzothiophene ring, and a pyridine ring. Examples of the heterocycle having no aromaticity include a tetrahydropyran ring, lactone rings, sultone rings, and a decahydroisoquinoline ring. The heterocycle in the heterocyclic group is preferably a furan ring, a thiophene ring, a pyridine ring, or a decahydroisoquinoline ring.

The above cyclic organic group may have a substituent. Examples of the substituent include alkyl groups (which may be linear or branched and have preferably 1 to 12 carbon atoms), cycloalkyl groups (which may be monocyclic, polycyclic, or spirocyclic and have preferably 3 to 20 carbon atoms), aryl groups (having preferably 6 to 14 carbon atoms), a hydroxy group, alkoxy groups, ester groups, amido groups, urethane groups, ureide groups, thioether groups, sulfonamido groups, and sulfonate groups. Carbon included in the cyclic organic group (carbon contributing to the formation of the ring) may be carbonyl carbon. Two or more substituents may be bonded together to form a ring. For example, two alkoxy groups or a hydroxy group and an alkoxy group may be bonded together to form a ring having a cyclic acetal structure. This ring may have a substituent. Examples of the substituent include alkyl groups (having 1 to 4 carbon atoms), halogen atoms, a hydroxy group, alkoxy groups (having 1 to 4 carbon atoms), a carboxyl group, and alkoxycarbonyl groups (having 2 to 6 carbon atoms).

The anion represented by formula (AN1) is preferably SO₃⁻—CF₂—CH₂—OCO-(L)_{q′}-W, SO₃⁻—CF₂—CHF—CH₂—OCO-(L)_{q}-W, SO₃⁻—CF₂—COO-(L)_{q′}-W, SO₃⁻—CF₂—CF₂—CH₂—CH₂-(L)_{q}-W, or SO₃⁻—CF₂—CH(CF₃)—OCO-(L)_{q′}-W. L, q, and W are the same as those in formula (AN1). Q′ represents an integer of from 0 to 10.

The anion represented by formula (AN1) is preferably any of the following modes (AN2) and (AN3).

Mode (AN2): In formula (AN1), o represents 2, and p represents 0. Two Xf's bonded to a carbon atom bonded directly to —SO₃⁻ (this carbon atom is hereinafter referred to also as a “carbon atom Z1”) each independently represent a hydrogen atom or an organic group having no fluorine atom. Two Xf's bonded to a carbon atom adjacent to the above carbon atom (this carbon atom is hereinafter referred to also as a “carbon atom Z2”) each independently represent a hydrogen atom or an organic group. Preferred modes of q, L, and W are the same as those described above.

The two Xf's bonded to the carbon atom Z1 are each preferably a hydrogen atom.

It is preferable that at least one of the two Xf's bonded to the carbon atom Z2 is a fluorine atom or an organic group having a fluorine atom. It is more preferable that both of the two Xf's bonded to the carbon atom Z2 are each a fluorine atom or an organic group having a fluorine atom. It is still more preferable that both are each an alkyl group substituted with fluorine.

Mode (AN3): In formula (AN1), one of the two Xf's represents a fluorine atom or an alkyl group substituted with at least one fluorine atom, and the other one represents a hydrogen atom or an organic group having no fluorine atom. Preferred modes of o, p, q, R₄, R₅, L, and W are the same as those described above.

The non-nucleophilic anion may be a benzenesulfonate anion and is preferably a benzenesulfonate anion substituted with a branched alkyl group or a cycloalkyl group.

The non-nucleophilic anion is also preferably an aromatic sulfonate anion represented by the following formula (AN5).

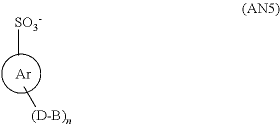

In formula (AN5), Ar represents an aryl group (such as a phenyl group) and may further have a substituent other than a sulfonate anion and the -(D-B) group. Examples of the substituent include a fluorine atom and a hydroxy group.

n represents an integer of 0 or more. N is preferably 1 to 4, more preferably 2 to 3, and still more preferably 3.

D represents a single bond or a divalent linking group. Examples of the divalent linking group include ether groups, thioether groups, a carbonyl group, sulfoxide groups, a sulfone group, sulfonate groups, ester groups, and groups formed by combining two or more of these groups.

B represents a hydrocarbon group.

Preferably, B has an aliphatic hydrocarbon structure. B is more preferably an isopropyl group, a cyclohexyl group, or an aryl group (such as a tricyclohexylphenyl group) optionally having a substituent.

B may further have a substituent represented by “-(L)_{q}-W.” L, q, and W have the same meanings as L, q, and W in formula (AN1) above, and their specific examples and preferred ranges are also the same as those of L, q, and W in formula (AN1).

The non-nucleophilic anion is also preferably a disulfonamide anion.

The disulfonamide anion is, for example, an anion represented by N—(SO₂—R^{q})₂.

Here, R^{q} represents an alkyl group optionally having a substituent and is preferably a fluoroalkyl group and more preferably a perfluoroalkyl group. Two R^{q}'s may be bonded together to form a ring. The group formed by bonding two R^{q}'s is preferably an alkylene group optionally having a substituent, more preferably a fluoroalkylene group, and still more preferably a perfluoroalkylene group. The number of carbon atoms in the alkylene group is preferably 2 to 4.

Other examples of the non-nucleophilic anion include anions represented by the following formulas (d1-1) to (d1-4).

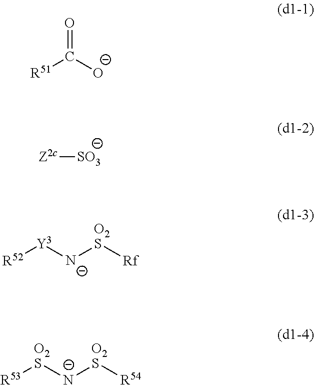

In formula (d1-1), R⁵¹ represents a hydrocarbon group (e.g., an aryl group such as a phenyl group) optionally having a substituent (e.g., a hydroxy group).

In formula (d1-2), Z^{2c} represents a hydrocarbon group having 1 to 30 carbon atoms and optionally having a substituent (provided that the carbon atom adjacent to S is not substituted with a fluorine atom).

The hydrocarbon group represented by Z^{2c} may be linear or branched and may have a ring structure. A carbon atom in the hydrocarbon group (preferably, a carbon atom serving as a ring member atom when the hydrocarbon group has a ring structure) may be carbonyl carbon (—CO—). Examples of the hydrocarbon group include a group having a norbornyl group optionally having a substituent. A carbon atom included in the norbornyl group may be carbonyl carbon.

It is preferable that “Z^{2c}—SO₃—” in formula (d1-2) differs from the anions represented by formulas (AN4), (AN1), and (AN5) above. For example, Z^{2c} differs from an aryl group. For example, in Z^{2c}, the atoms at the α- and β-positions with respect to —SO₃— are each preferably an atom different from a carbon atom having a fluorine atom as a substituent. For example, in Z^{2c}, the atom at the α-position and/or the atom at the β-position with respect to —SO₃⁻ is preferably a ring member atom of the cyclic group.

In formula (d1-3), R⁵² represents an organic group (preferably a hydrocarbon group having a fluorine atom), and Y³ represents a linear, branched, or cyclic alkylene group, an arylene group, or a carbonyl group. Rf represents a hydrocarbon group.

In formula (d1-4), R⁵³ to R¹⁴ each represent an organic group (preferably a hydrocarbon group having a fluorine atom). R⁵³ to R¹⁴ may be bonded together to form a ring.

One type of organic anion may be used alone, or two or more types of organic anions may be used.

Preferably, the resist composition includes two or more compounds (B), or the compound (B) is at least one selected from the group consisting of compounds (I) and (II) below.

### <Compound (I) and Compound (II)>

It is also preferable that the compound (B) is at least one selected from the group consisting of the compound (I) below and the compound (II) below.

### (Compound (I))

The compound (I) is a compound having at least one structural moiety X described below and at least one structural moiety Y described below and is a compound that generates an acid including a first acidic moiety described below and derived from the structural moiety X and a second acidic moiety described below and derived from the structural moiety Y when irradiated with actinic rays or radiation.

Structural moiety X: A structural moiety including an anionic moiety A₁⁻ and a cationic moiety M₁⁺ and forms the first acidic moiety represented by HA₁ when irradiated with actinic rays or radiation.

Structural moiety Y: A structural moiety including an anionic moiety A₂⁻ and a cationic moiety M₂⁺ and forms the second acidic moiety represented by HA₂ when irradiated with actinic rays or radiation.

Preferably, the cationic moiety M₁⁺ and the cationic moiety M₂⁺ each independently represent an organic cation, and their specific examples and preferred modes are the same as those of the organic cation represented by M⁺ described above.

The compound (I) satisfies the following condition I.

Condition I: A compound PI formed by replacing each of the cationic moiety M₁⁺ in the structural moiety X in the compound (I) and the cationic moiety M₂⁺ in the structural moiety Y with H⁺ has an acid dissociation constant a1 derived from the acidic moiety represented by HA₁ formed by replacing the cationic moiety M₁⁺ in the structural moiety X with H⁺ and an acid dissociation constant a2 derived from the acidic moiety represented by HA₂ formed by replacing the cationic moiety M₂⁺ in the structural moiety Y with H⁺, and the acid dissociation constant a2 is larger than the acid dissociation constant a1.

The condition I will be specifically described.

When the compound (I) is a compound that generates an acid having one first acidic moiety derived from the structural moiety X and one second acidic moiety derived from the structural moiety Y, the compound PI corresponds to a “compound having HA₁ and HA₂.”

The acid dissociation constant a1 and the acid dissociation constant a2 of this compound PI will be specifically described. When the acid dissociation constants of the compound PI are determined, the pKa when the compound PT becomes a “compound having A₁⁻ and HA₂” is the acid dissociation constant a1, and the pKa when the “compound having A₁⁻ and HA₂” becomes a “compound having A₁⁻ and A₂⁻” is the acid dissociation constant a2.

When the compound (I) is, for example, a compound that generates an acid having two first acidic moieties derived from the structural moieties X and one second acidic moiety derived from the structural moiety Y, the compound PI corresponds to a “compound having two HA₁'s and one HA₂.”

When the acid dissociation constants of the compound PI are determined, the acid dissociation constant when the compound PI becomes a “compound having one A₁⁻, one HA₁, and one HA₂,” and the acid dissociation constant when the “compound having one A₁⁻, one HA₁, and one HA₂” becomes a “compound having two A₁⁻'s and one HA₂” each correspond to the acid dissociation constant a1. Moreover, the acid dissociation constant when the “compound having two A₁⁻'s and one HA₂” becomes a “compound having two A₁⁻'s and A₂⁻” corresponds to the acid dissociation constant a2. Specifically, such a compound PI has a plurality of acid dissociation constants derived from acidic moieties represented by HA₁ formed by replacing cationic moieties M₁⁺ in the structural moieties X with H⁺. In this case, the acid dissociation constant a2 is larger than the largest one of the plurality of acid dissociation constants a1. Let aa be the acid dissociation constant when the compound PI becomes the “compound having one A₁⁻, one HA₁, and one HA₂,” and ab be the acid dissociation constant when the “compound having one A₁⁻, one HA₁, and one HA₂” becomes the “compound having two A₁⁻'s and one HA₂.” Then aa and ab satisfy the relation aa<ab.

The acid dissociation constants a1 and a2 are determined by the acid dissociation constant measurement method described above.

The compound PI corresponds to the acid generated when the compound (I) is irradiated with actinic rays or radiation.

When the compound (I) has two or more structural moieties X, these structural moieties X may be the same or different. Moreover, two or more A₁⁻'s may be the same or different, and two or more M₁⁺'s may be the same or different.

In compound (I), A₁⁻ and A₂⁻ may be the same or different, and M₁⁺ and M₂⁺ may be the same or different. However, it is preferable that A₁⁻ and A₂⁻ are different from each other.

In the compound PI, the difference (absolute difference) between the acid dissociation constant a1 (when a plurality of acid dissociation constants a1 are present, the maximum value of the acid dissociation constants) and the acid dissociation constant a2 is preferably 0.1 or more, more preferably 0.5 or more, and still more preferably 1.0 or more. No particular limitation is imposed on the upper limit of the difference (absolute difference) between the acid dissociation constant a1 (when a plurality of acid dissociation constants a1 are present, the maximum value of the acid dissociation constants) and the acid dissociation constant a2, but the upper limit is, for example, 16 or less.

In the compound PI, the acid dissociation constant a2 is, for example, 20 or less and preferably 15 or less. The lower limit of the acid dissociation constant a2 is preferably −4.0 or more.

In the compound PI, the acid dissociation constant a1 is preferably 2.0 or less and more preferably 0 or less. The lower limit of the acid dissociation constant a1 is preferably −20.0 or more.

The anionic moiety A₁⁻ and the anionic moiety A₂ are each a structural moiety including a negatively charged atom or atomic group and are each, for example, a structural moiety selected from the group consisting of formulas (AA-1) to (AA-3) and formulas (BB-1) to (BB-6) shown below.

The anionic moiety A₁⁻ is preferably a moiety capable of forming an acidic moiety having a small acid dissociation constant, more preferably a moiety represented by any one of (AA-1) to (AA-3), and still more preferably a moiety represented by any one of formulas (AA-1) and (AA-3).

The anionic moiety A₂ is preferably a moiety capable of forming an acidic moiety having a larger acid dissociation constant than the anionic moiety A₁-, more preferably a moiety represented by any one of formulas (BB-1) to (BB-6), and still more preferably a moiety represented by any one of formulas (BB-1) and (BB-4).

In formulas (AA-1) to (AA-3) and formulas (BB-1) to (BB-6) below, * represents a bonding position.

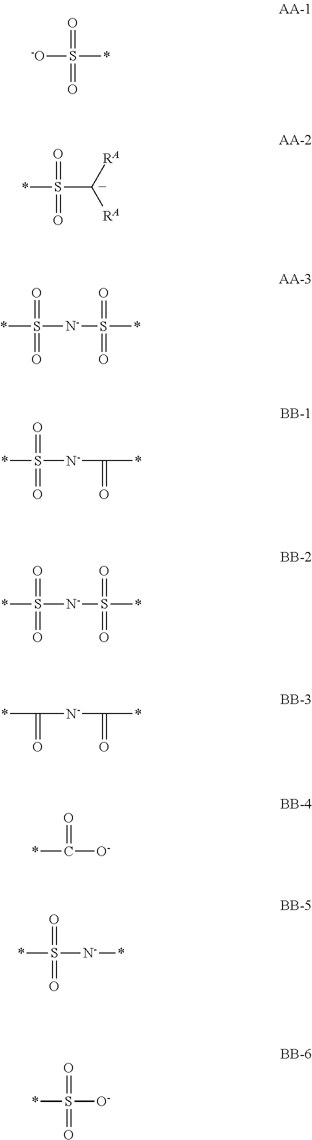

No particular limitation is imposed on the specific structure of the compound (I), and examples thereof include compounds represented by formulas (Ia-1) to (Ia-5) described below.

—Compound represented by formula (Ia-1)—

First, the compound represented by formula (Ia-1) will be described.

M₁₁⁺A₁₁⁻-L₁-A₁₂⁻M₁₂⁺ (Ia-1)

The compound represented by formula (Ia-1) generates an acid represented by HA₁₁-L₁-A₁₂H when irradiated with actinic rays or radiation.

In formula (Ia-1), M₁₁⁺ and M₁₂⁺ each independently represent an organic cation.

A₁₁⁻ and A₁₂⁻ each independently represent a monovalent anionic functional group.

L₁ represents a divalent linking group.

M₁₁⁺ and M₁₂⁺ may be the same or different.

A₁₁⁻ and A₁₂⁻ may be the same or different, but it is preferable that they differ from each other.

In a compound PIa (HA₁₁-L₁-A₁₂H) formed by replacing each of the cations represented by M₁₁⁺ and M₁₂⁺ in formula (Ia-1) above with H⁺, the acid dissociation constant a2 derived from the acidic moiety represented by A₁₂H is larger than the acid dissociation constant a1 derived from the acidic moiety represented by HA₁₁. Preferred values of the acid dissociation constants a1 and a2 are as described above. The compound PIa is the same as the acid generated from the compound represented by formula (Ia-1) upon irradiation with actinic rays or radiation.

At least one of M₁₁⁺, M₁₂⁺, A₁₁⁻, A₁₂⁻, or L₁ may have an acid-decomposable group as a substituent.

In formula (Ia-1), the organic cations represented by M₁₁⁺ and M₁₂⁺ are as described above.

The monovalent anionic functional group represented by A₁₁⁻ means a monovalent group including the anionic moiety A₁⁻ described above. The monovalent anionic functional group represented by A₁₂⁻ means a monovalent group including the anionic moiety A₂⁻ described above.

The monovalent anionic functional groups represented by A₁₁⁻ and A₁₂⁻ are each preferably a monovalent anionic functional group including the anionic moiety represented by any of formulas (AA-1) to (AA-3) and formulas (BB-1) to (BB-6) described above and more preferably a monovalent anionic functional group selected from the group consisting of formulas (AX-1) to (AX-3) and formulas (BX-1) to (BX-7). In particular, the monovalent anionic functional group represented by A₁₁⁻ is preferably a monovalent anionic functional group represented by any of formulas (AX-1) to (AX-3). In particular, the monovalent anionic functional group represented by A₁₂⁻ is preferably a monovalent anionic functional group represented by any of formulas (BX-1) to (BX-7) and more preferably a monovalent anionic functional group represented by any of formulas (BX-1) to (BX-6).

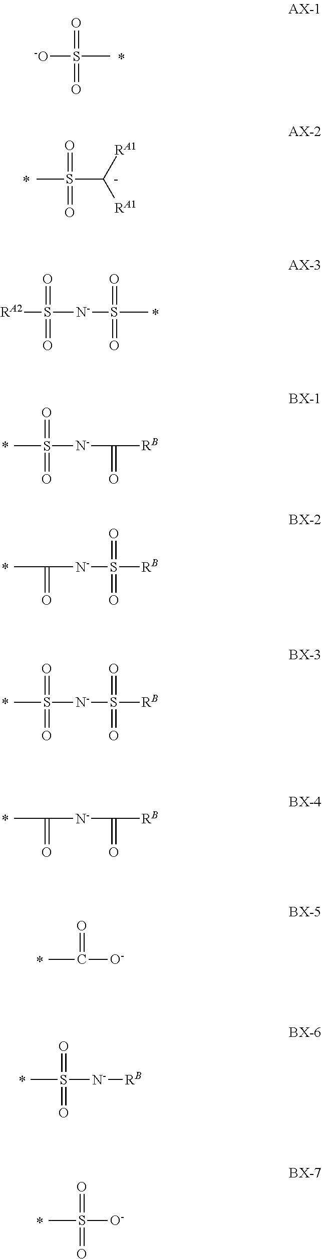

In formulas (AX-1) to (AX-3), R^{A1} and R^{A2} each independently represent a monovalent organic group. * represents a bonding position.

Examples of the monovalent organic group represented by R^{A1} include a cyano group, a trifluoromethyl group, and a methanesulfonyl group.

The monovalent organic group represented by R^{A2} is preferably a linear, branched, or cyclic alkyl group or an aryl group.

The number of carbon atoms in the alkyl group is preferably 1 to 15, more preferably 1 to 10, and still more preferably 1 to 6.

The alkyl group may have a substituent. The substituent is preferably a fluorine atom or a cyano group and more preferably a fluorine atom. When the alkyl group has a fluorine atom as a substituent, the alkyl group may be a perfluoroalkyl group.

The aryl group is preferably a phenyl group or a naphthyl group and more preferably a phenyl group.

The aryl group may have a substituent. The substituent is preferably a fluorine atom, an iodine atom, a perfluoroalkyl group (for example, having preferably 1 to 10 carbon atoms and more preferably 1 to 6 carbon atoms), or a cyano group and is more preferably a fluorine atom, an iodine atom, or a perfluoroalkyl group.

In formulas (BX-1) to (BX-4) and (BX-6), R^{B} represents a monovalent organic group. * represents a bonding position.

The monovalent organic group represented by R^{B} is preferably a linear, branched, or cyclic alkyl group or an aryl group.

The number of carbon atoms in the alkyl group is preferably 1 to 15, more preferably 1 to 10, and still more preferably 1 to 6.

The alkyl group may have a substituent. No particular limitation is imposed on the substituent, but the substituent is preferably a fluorine atom or a cyano group and more preferably a fluorine atom. When the alkyl group has a fluorine atom as a substituent, the alkyl group may be a perfluoroalkyl group.

When a carbon atom in the alkyl group at a bonding position (for example, the carbon atom in the alkyl group that is bonded directly to —CO— in any of formulas (BX-1) and (BX-4), the carbon atom shown in the alkyl group that is bonded directly to —SO₂— in any of formulas (BX-2) and (BX-3), or the carbon atom shown in the alkyl group that is bonded directly to N⁻ in formula (BX-6)) has a substituent, it is also preferable that the substituent differs from a fluorine atom and a cyano group.

In the alkyl group, a carbon atom may be replaced with carbonyl carbon.

The aryl group is preferably a phenyl group or a naphthyl group and more preferably a phenyl group.

The aryl group may have a substituent. The substituent is preferably a fluorine atom, an iodine atom, a perfluoroalkyl group (for example, having preferably 1 to 10 carbon atoms and more preferably 1 to 6 carbon atoms), a cyano group, an alkyl group (for example, having preferably 1 to 10 carbon atoms and more preferably 1 to 6 carbon atoms), an alkoxy group (for example, having preferably 1 to 10 carbon atoms and more preferably 1 to 6 carbon atoms), or an alkoxycarbonyl group (for example, having preferably 2 to 10 carbon atoms and more preferably 2 to 6 carbon atoms) and more preferably a fluorine atom, an iodine atom, a perfluoroalkyl group, an alkyl group, an alkoxy group, or an alkoxycarbonyl group.

In formula (Ia-1), no particular limitation is imposed on the divalent linking group represented by L₁, and examples of L₁ include —CO—, —NR—, —O—, —S—, —SO—, —SO₂—, alkylene groups (which may be linear or branched and have preferably 1 to 6 carbon atoms), cycloalkylene groups (having preferably 3 to 15 carbon atoms), alkenylene groups (having preferably 2 to 6 carbon atoms), divalent aliphatic heterocyclic groups (preferably 5- to 10-membered rings, more preferably 5- to 7-membered rings, and still more preferably 5- to 6-membered rings, each of which has at least one N atom, O atom, S atom, or Se atom in the ring structure), divalent aromatic heterocyclic groups (preferably 5- to 10-membered rings, more preferably 5- to 7-membered rings, and still more preferably 5- to 6-membered rings, each of which has at least one N atom, O atom, S atom, or Se atom in the ring structure), divalent aromatic hydrocarbon ring groups (preferably 6- to 10-membered rings and more preferably 6-membered rings), and divalent linking groups formed by combining a plurality of groups selected from the above groups. R is a hydrogen atom or a monovalent organic group. No particular limitation is imposed on the monovalent organic group, but the monovalent organic group is preferably, for example, an alkyl group (having preferably 1 to 6 carbon atoms).

The alkylene, cycloalkylene, alkenylene, divalent aliphatic heterocyclic, divalent aromatic heterocyclic, and divalent aromatic hydrocarbon ring groups described above may each have a substituent. Examples of the substituent include halogen atoms (preferably a fluorine atom).

In particular, the divalent linking group represented by L₁ is preferably a divalent linking group represented by formula (L1).

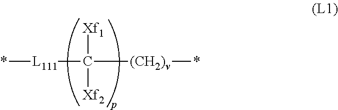

In formula (L1), L₁₁₁ represents a single bond or a divalent linking group.

No particular limitation is imposed on the divalent linking group represented by L₁₁₁, and examples of the divalent linking group include —CO—, —NH—, —O—, —SO—, —SO₂—, alkylene groups (which may be linear or branched and have preferably 1 to 6 carbon atoms) optionally having a substituent, cycloalkylene groups (having preferably 3 to 15 carbon atoms) optionally having a substituent, aryl groups (having preferably 6 to 10 carbon atoms) optionally having a substituent, and divalent linking groups formed by combining a plurality of groups selected from the above groups. No particular limitation is imposed on the substituent, and examples thereof include halogen atoms.

p represents an integer of from 0 to 3 and preferably represents an integer of from 1 to 3.

v represents an integer of 0 or 1.

Xf₁'s each independently represent a fluorine atom or an alkyl group substituted with at least one fluorine atom. The number of carbon atoms in the alkyl group is preferably 1 to 10 and more preferably 1 to 4. The alkyl group substituted with at least one fluorine atom is preferably a perfluoroalkyl group.

Xf₂'s each independently represent a hydrogen atom, an alkyl group optionally having a fluorine atom as a substituent, or a fluorine atom. The number of carbon atoms in the alkyl group is preferably 1 to 10 and more preferably 1 to 4. In particular, each Xf₂ represents preferably a fluorine atom or an alkyl group substituted with at least one fluorine atom and more preferably a fluorine atom or a perfluoroalkyl group.

In particular, Xf₁'s and Xf₂'s each independently represent preferably a fluorine atom or a perfluoroalkyl group having 1 to 4 carbon atoms and more preferably a fluorine atom or CF₃.

In particular, it is still more preferable that Xf₁'s and Xf₂'s are each a fluorine atom.

* represents a bonding position.

When L₁ in formula (Ia-1) represents a divalent linking group represented by formula (L1), it is preferable that the direct bond (*) on the L₁₁₁ side in formula (L1) is bonded to A₁₂⁻in formula (Ia-1).

### —Compounds Represented be Formulas (Ia-2) to (Ia-4)—

Next, compounds represented by formulas (Ia-2) to (Ia-4) will be described.

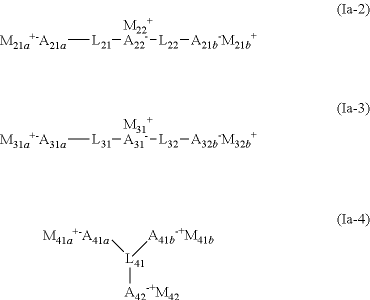

In formula (Ia-2), A₂₁ₐ⁻ and A_{21b}⁻ each independently represent a monovalent anionic functional group. Each of the monovalent anionic functional groups represented by A₂₁ₐ⁻ and A₂₁ₐ⁻ means a monovalent group including the above-described anionic moiety A₁⁻. No particular limitation is imposed on the monovalent anionic functional groups represented by A₂₁ₐ⁻ and A_{21b}⁻, but A₂₁ₐ⁻ and A_{21b}⁻ are each, for example, a monovalent anionic functional group selected from the group consisting of formulas (AX-1) to (AX-3) described above.

A₂₂⁻ represents a divalent anionic functional group. The divalent anionic functional group represented by A₂₂⁻ means a divalent groups including the anionic moiety A₂⁻ described above. Examples of the divalent anionic functional group represented by A₂₂⁻ include divalent anionic functional groups represented by the following formulas (BX-8) to (BX-11).

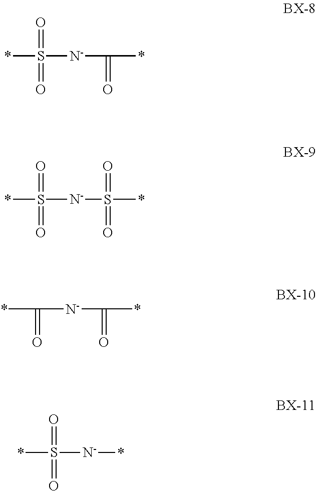 [SMILES: *C(=O)[N-]S(*)(=O)=O ; *S(=O)(=O)[N-]S(*)(=O)=O ; *C(=O)[N-]C(*)=O ; *[N-]S(*)(=O)=O]

M₂₁ₐ⁺, M_{21b}⁺, and M₂₂⁺ each independently represent an organic cation. The definitions of the organic cations represented by M₂₁ₐ⁺, M_{21b}⁺, and M₂₂⁺ are the same as that of M₁₁⁺ described above, and their preferred modes are also the same as those of M₁₁⁺.

L₂₁ and L₂₂ each independently represent a divalent organic group.

In a compound PIa-2 formed by replacing each of the organic cations represented by M₂₁ₐ⁺, M_{21b}⁺, and M₂₂⁺ in formula (Ia-2) with H⁺, the acid dissociation constant a2 derived from an acidic moiety represented by A₂₂H is larger than the acid dissociation constant a1-1 derived from an acidic moiety represented by A₂₁ₐH and the acid dissociation constant a1-2 derived from an acidic moiety represented by A_{21b}H. The acid dissociation constant a1-1 and the acid dissociation constant a1-2 each correspond to the acid dissociation constant a1 described above.

A₂₁ₐ⁻ and A_{21b}⁻ may be the same or different. M₂₁ₐ⁺, M_{21b}⁺, and M₂₂⁺ may be the same or different.

At least one of M₂₁ₐ⁺, M_{21b}⁺, M₂₂⁺, A₂₁ₐ⁻, A_{21b}⁻, L₂₁, or L₂₂ may have an acid-decomposable group as a substituent.

In formula (Ia-3), A₃₁ₐ⁻ and A₃₂⁻ each independently represent a monovalent anionic functional group. The definition of the monovalent anionic functional group represented by A₃₁ₐ⁻ is the same as those of A₂₁ₐ⁻ and A_{21b}⁻ in formula (Ia-2), and its preferred mode is also the same as those of A₂₁ₐ⁻ and A_{21b}⁻.

The monovalent anionic functional group represented by A₃₂⁻ means a monovalent group including the anionic moiety A₂⁻ described above. No particular limitation is imposed on the monovalent anionic functional group represented by A₃₂⁻, and A₃₂⁻ is, for example, a monovalent anionic functional group selected from the group consisting of formulas (BX-1) to (BX-7) described above.

A_{31b}⁻ represents a divalent anionic functional group. The divalent anionic functional group represented by A_{31b}⁻ means a divalent group including the anionic moiety A₁⁻ described above. Examples of the divalent anionic functional group represented by A_{31b}⁻ include a divalent anionic functional group represented by the following formula (AX-4).

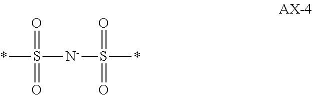 [SMILES: *S(=O)(=O)[N-]S(*)(=O)=O]

M₃₁ₐ⁺, M_{31b}⁺, and M₃₂⁺ each independently represent a monovalent organic cation. The definitions of the organic cations represented by M₃₁ₐ⁺, M_{31b}⁺, and M₃₂⁺ are the same are that of M₁₁⁺ described above, and their preferred modes are also the same as that of M₁₁⁺.

L₃₁ and L₃₂ each independently represent a divalent organic group.

In a compound PIa-3 formed by replacing each of the organic cations represented by M₃₁ₐ⁺, M_{31b}⁺, and M₃₂⁺ in formula (Ia-3) with H⁺, the acid dissociation constant a2 derived from an acidic moiety represented by A₃₂H is larger than the acid dissociation constant a1-3 derived from an acidic moiety represented by A₃₁ₐH and the acid dissociation constant a1-4 derived from an acidic moiety represented by A_{31b}H. The acid dissociation constant a1-3 and the acid dissociation constant a1-4 each correspond to the acid dissociation constant a1 described above.

A₃₁ₐ⁻ and A₃₂⁻ may be the same or different. M₃₁ₐ⁺, M_{31b}⁺, and M₃₂⁺ may be the same or different.

At least one of M₃₁ₐ⁺, M_{31b}⁺, M₃₂⁺, A₃₁ₐ⁻, A₃₂⁻, L₃₁, or L₃₂ may have an acid-decomposable group as a substituent.

In formula (Ia-4), A₄₁ₐ⁻, A_{41b}⁻, and A₄₂⁻ each independently represent a monovalent anionic functional group. The definitions of the monovalent anionic functional groups represented by A₄₁ₐ⁻ and A_{41b}⁻ are the same as the definitions of A₂₁ₐ⁻ and A_{21b}⁻ in formula (Ia-2) described above. The definition of the monovalent anionic functional group represented by A₄₂⁻ is the same as that of A₃₂⁻ in formula (Ia-3) described above, and its preferred mode is also the same as that of A₃₂⁻.

M₄₁ₐ⁺, M_{41b}⁺, and M₄₂⁺ each independently represent an organic cation.

L₄₁ represents a trivalent organic group.

In a compound PIa-4 formed by replacing each of the organic cations represented by M₄₁ₐ⁺, M_{41b}⁺, and M₄₂⁺ in formula (Ia-4) with H⁺, the acid dissociation constant a2 derived from an acidic moiety represented by A₄₂H is larger than the acid dissociation constant a1-5 derived from an acidic moiety represented by A₄₁ₐH and the acid dissociation constant a1-6 derived from an acidic moiety represented by A_{41b}H. The acid dissociation constant a1-5 and the acid dissociation constant a1-6 each correspond to the acid dissociation constant a1 described above.

A₄₁ₐ⁻, A_{41b}⁻, and A₄₂⁻ may be the same or different. M₄₁ₐ⁺, M_{41b}⁺, and M₄₂⁺ may be the same or different.

At least one of M₄₁ₐ⁺, M_{41b}⁺, M₄₂⁺, A₄₁ₐ⁻, A_{41b}⁻, A₄₂⁻, or L₄₁ may have an acid-decomposable group as a substituent.

No particular limitation is imposed on the divalent organic groups represented by L₂₁ and L₂₂ in In formula (Ia-2) and L₃₁ and L₃₂ in formula (Ia-3), and examples thereof include —CO—, —NR—, —O—, —S—, —SO—, —SO₂—, alkylene groups (which may be linear or branched and have preferably 1 to 6 carbon atoms), cycloalkylene groups (having preferably 3 to 15 carbon atoms), alkenylene groups (having preferably 2 to 6 carbon atoms), divalent aliphatic heterocyclic groups (preferably 5- to 10-membered rings, more preferably 5- to 7-membered rings, and still more preferably 5- to 6-membered rings, each of which has at least one N atom, O atom, S atom, or Se atom in the ring structure), divalent aromatic heterocyclic groups (preferably 5- to 10-membered rings, more preferably 5- to 7-membered rings, and still more preferably 5- to 6-membered rings, each of which has at least one N atom, O atom, S atom, or Se atom in the ring structure), divalent aromatic hydrocarbon ring groups (preferably 6- to 10-membered rings and more preferably 6-membered rings), and divalent organic groups formed by combining a plurality of groups selected from the above groups. Examples of R include a hydrogen atom and monovalent organic groups. No particular limitation is imposed on the monovalent organic group, but the monovalent organic group is, for example, preferably an alkyl group (having preferably 1 to 6 carbon atoms).

The alkylene, cycloalkylene, alkenylene, divalent aliphatic heterocyclic, divalent aromatic heterocyclic, and divalent aromatic hydrocarbon ring groups described above may each have a substituent. Examples of the substituent include halogen atoms (preferably a fluorine atom).

Preferably, the divalent organic groups represented by L₂₁ and L₂₂ in formula (Ia-2) and L₃₁ and L₃₂ in formula (Ia-3) are each, for example, a divalent organic group represented by formula (L2) below.

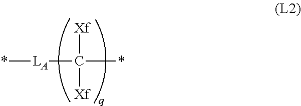

In formula (L2), q represents an integer of from 1 to 3. * represents a bonding position.

Xf's each independently represent a fluorine atom or an alkyl group substituted with at least one fluorine atom. The number of carbon atoms in the alkyl group is preferably 1 to 10 and more preferably 1 to 4. The alkyl group substituted with at least one fluorine atom is preferably a perfluoroalkyl group.

Xf's are each preferably a fluorine atom or a perfluoroalkyl group having 1 to 4 carbon atoms and more preferably a fluorine atom or CF₃. In particular, it is more preferable that both Xf's are fluorine atoms.

L_{A} represents a single bond or a divalent linking group.

No particular limitation is imposed on the divalent linking group represented by L_{A}, and examples thereof include —CO—, —O—, —SO—, —SO₂—, alkylene groups (which may be linear or branched and have preferably 1 to 6 carbon atoms), cycloalkylene groups (having preferably 3 to 15 carbon atoms), divalent aromatic hydrocarbon ring groups (preferably 6- to 10-membered rings and more preferably 6-membered rings), and divalent linking groups formed by combining a plurality of groups selected from the above groups.

The alkylene, cycloalkylene, and divalent aromatic hydrocarbon ring groups described above may each have a substituent. Examples of the substituent include halogen atoms (preferably a fluorine atom).

Examples of the divalent organic group represented by formula (L2) include *—CF₂—*, *—CF₂—CF₂—*, *—CF₂—CF₂—CF₂—*, *-Ph-O—SO₂—CF₂—*, *-Ph-O—SO₂—CF₂—CF₂—*, *-Ph-O—SO₂—CF₂—CF₂—CF₂—*, and *-Ph-OCO—CF₂—*. Ph is a phenylene group optionally having a substituent and is preferably a 1,4-phenylene group. No particular limitation is imposed on the substituent, but the substituent is preferably an alkyl group (for example, having preferably 1 to 10 carbon atoms and more preferably 1 to 6 carbon atoms), an alkoxy group (for example, having preferably 1 to 10 carbon atoms and more preferably 1 to 6 carbon atoms), or an alkoxycarbonyl group (for example, having preferably 2 to 10 carbon atoms and more preferably 2 to 6 carbon atoms).

When L₂₁ and L₂₂ in formula (Ia-2) each represent the divalent organic group represented by formula (L2), it is preferable that the direct bond (*) on the L_{A} side in formula (L2) is bonded to A₂₁ₐ⁻ or A₂₁⁻ in formula (Ia-2).

When L₃₁ and L₃₂ in formula (Ia-3) each represent the divalent organic group represented by formula (L2), it is preferable that the direct bond (*) on the L_{A} side in formula (L2) is bonded to A₃₁ₐ⁻ or A₃₂⁻ in formula (Ia-3).

### —Compound Represented by Formula (Ia-5)—

Next, formula (Ia-5) will be described.

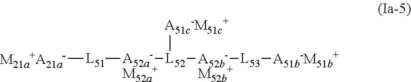

In formula (Ia-5), A₅₁ₐ⁻, A_{51b}⁻, and A_{51c}⁻ each independently represent a monovalent anionic functional group. Each of the monovalent anionic functional groups represented by A₅₁ₐ⁻, A_{51b}⁻, and A_{51c}⁻ means a monovalent group including the anionic moiety A₁⁻ described above. No particular limitation is imposed on the monovalent anionic functional groups represented by A₅₁ₐ⁻, A_{51b}⁻, and A_{51c}⁻, but each of these monovalent anionic functional groups is, for example, a monovalent anionic functional group selected from the group consisting of formulas (AX-1) to (AX-3) described above.

A₅₂ₐ⁻ and A_{52b}⁻ each represent a divalent anionic functional group. Each of the divalent anionic functional groups represented by A₅₂ₐ⁻ and A_{52b}⁻ means a divalent group including the anionic moiety A₂⁻ described above. The divalent anionic functional groups represented by A₅₂ₐ⁻ and A_{52b}⁻ are each, for example, a divalent anionic functional groups selected from the group consisting of formulas (BX-8) to (BX-11) described above.

M₅₁ₐ⁺, M_{51b}⁺, M_{51c}⁺, M₅₂ₐ⁺, and M_{52b}⁺ each independently represent an organic cation. The definitions of the organic cations represented by M₅₁ₐ⁺, M_{51b}⁺, M_{51c}⁺, M₅₂ₐ⁺, and M_{52b}⁺ are the same as the definition of M₁₁⁺ described above, and their preferred modes are also the same as that of M₁₁⁺.

L₅₁ and L₅₃ each independently represent a divalent organic group. The definitions of the divalent organic groups represented by L₅₁ and L₅₃ are the same as those of L₂₁ and L₂₂ in formula (Ia-2) described above, and their preferred modes are also the same as those of L₂₁ and L₂₂.

L₅₂ represents a trivalent organic group. The definition of the trivalent organic group represented by L₅₂ is the same as that of L₄₁ in formula (Ia-4) described above, and its preferred mode is also the same as that of L₄₁.

In a compound PIa-5 formed by replacing each of the organic cations represented by M₅₁ₐ⁺, M_{51b}⁺, M_{51c}⁺, M₅₂ₐ⁺, and M_{52b}⁺ in formula (Ia-5) with H⁺, the acid dissociation constant a2-1 derived from an acidic moiety represented by A₅₂ₐH and the acid dissociation constant a2-2 derived from an acidic moiety represented by A_{52b}H are larger than the acid dissociation constant a1-1 derived from an acidic moiety represented by A₅₁ₐH, the acid dissociation constant a1-2 derived from an acidic moiety represented by A_{51b}H, and the acid dissociation constant a1-3 derived from an acidic moiety represented by A_{51c}H. The acid dissociation constants a1-1 to a1-3 each correspond to the acid dissociation constant a1 described above, and the acid dissociation constants a2-1 and a2-2 each correspond to the acid dissociation constant a2 described above.

A₅₁ₐ⁻, A_{51b}⁻, and A_{51c}⁻ may be the same or different. A₅₂ₐ⁻ and A_{52b}⁻ may be the same or different. M₅₁ₐ⁺, M_{51b}⁺, M_{51c}⁺, M₅₂ₐ⁺, and M_{52b}⁺ may be the same or different.

At least one of M_{51b}⁺, M_{51c}⁺, M₅₂ₐ⁺, M_{52b}⁺, A₅₁ₐ⁻, A_{51b}⁻, A_{51c}⁻, L₅₁, L₅₂, or L₅₃ may have an acid-decomposable group as a substituent.

### (Compound (II))

The compound (II) is a compound that has two or more structural moieties X described above and at least one structural moiety Z described below and is a compound that generates an acid including two or more first acidic moieties derived from the structural moieties X and the structural moiety Z when irradiated with actinic rays or radiation.

### Structural Moiety Z: Non-Ionic Moiety Capable of Neutralizing Acid

The definition of the structural moiety X in the compound (II) and the definitions of A₁⁻ and M₁⁺ are the same as that of the structural moiety X in the compound (I) described above and those of A₁⁻ and M₁⁺ in the structural moiety X in the compound (I) described above, and their preferred modes are also the same as those of the compound (I).

In a compound PII formed by replacing each of the cationic moieties M₁⁺ in the structural moieties X in the compound (II) with H⁺, a preferred range of the acid dissociation constant a1 derived from an acidic moiety represented by HA₁ formed by replacing the cationic moiety M₁⁺ in one of the structural moieties X with H⁺ is the same as that of the acid dissociation constant a1 in the compound PI.

When the compound (II) is, for example, a compound that generates an acid having two first acidic moieties derived from the structural moieties X and the structural moiety Z, the compound PII corresponds to a “compound having two HA₁s.” When the acid dissociation constants of the compound PII are determined, the acid dissociation constant when the compound PII becomes a “compound having one A₁⁻ and one HA₁” and the dissociation constant when the “compound having one A₁⁻ and one HA₁” becomes a “compound having two A₁⁻'s” each correspond to the acid dissociation constant a1.

The acid dissociation constant a1 is determined by the acid dissociation constant measurement method described above.

The compound PII corresponds to an acid generated when the compound (II) is irradiated with actinic rays or radiation.

The two or more structural moieties X may be the same or different. The two or more A₁⁻'s may be the same or different, and the two or more M₁⁺'s may be the same or different.

No particular limitation is imposed on the non-ionic moiety that is in the structural moiety Z and capable of neutralizing an acid, and the non-ionic moiety is, for example, preferably a moiety including a functional group having an electron or a group capable of electrostatically interacting with a proton.

Examples of the functional group having an electron or a group capable of electrostatically interacting with a proton include a functional group having a macrocyclic structure such as a cyclic polyether and a functional group having a nitrogen atom having an unshared electron pair not contributing to π-conjugation. The nitrogen atom having an unshared electron pair not contributing to π-conjugation is, for example, a nitrogen atom having a partial structure represented by any of the following formulas.

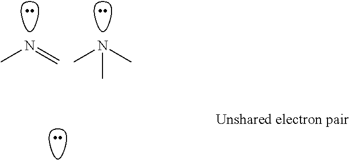

Examples of the partial structure of the functional group having an electron or a group capable of electrostatically interacting with a proton include crown ether structures, azacrown ether structures, primary to tertiary amine structures, a pyridine structure, an imidazole structure, and a pyrazine structure. Of these, primary to tertiary amine structures are preferred.

No particular limitation is imposed on the compound (II), and examples thereof include compounds represented by the following formulas (IIa-1) and (IIa-2).

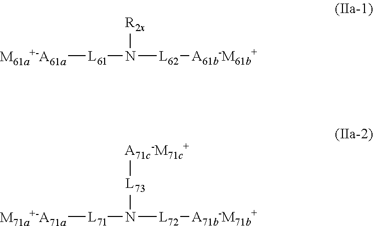

In formula (IIa-1), the definitions of A₆₁ₐ⁻ and A_{61b}⁻ are each the same as that of A₁₁⁻ in formula (Ia-1) above, and their preferred modes are also the same as that of A₁₁⁻. The definitions of M₆₁ₐ⁺ and M_{61b}⁺ are each the same as that of M₁₁⁺ in formula (Ia-1), and their preferred modes are also the same as that of M₁₁⁺.

In formula (IIa-1), the definitions of L₆₁ and L₆₂ are each the same as that of L₁ in formula (Ia-1) above, and their preferred modes are also the same as that of L₁.

In formula (IIa-1), R_{2X} represents a monovalent organic group. No particular limitation is imposed on the monovalent organic group represented by R_{2X}, and examples thereof include alkyl groups (which have preferably 1 to 10 carbon atoms and may be linear or branched), cycloalkyl groups (having preferably 3 to 15 carbon atoms), and alkenyl groups (having preferably 2 to 6 carbon atoms). In these groups, —CH₂— may be replaced with one or a combination of two or more selected from the group consisting of —CO—, —NH—, —O—, —S—, —SO—, and —SO₂—.

The above alkylene, cycloalkylene, and alkenylene groups may each have a substituent. No particular limitation is imposed on the substituent, and examples thereof include halogen atoms (preferably a fluorine atom).

In a compound PIIa-1 formed by replacing each of the organic cations represented by M₆₁ₐ⁺ and M₆₁⁺ in formula (IIa-1) with H⁺, the acid dissociation constant a1-7 derived from an acidic moiety represented by A₆₁ₐH and the acid dissociation constant a1-8 derived from an acidic moiety represented by A_{61b}H each correspond to the acid dissociation constant a1 described above.

The compound PIIa-1 formed by replacing each of the cationic moieties M₆₁ₐ⁺ and M_{61b}⁺ in the structural moieties X in the formula (IIa-1) corresponds to HA₆₁ₐ-L₆₁-N(R_{2X})-L₆₂-A_{61b}H. The compound PIIa-1 is the same as the acid generated from the compound represented by formula (IIa-1) upon irradiation with actinic rays or radiation.

At least one of M₆₁ₐ⁺, M_{61b}⁺, A₆₁ₐ⁻, A_{61b}⁻, L₆₁, L₆₂, or R_{2X} may have an acid-decomposable group as a substituent.

The definitions of A₇₁ₐ⁻, A_{71b}⁻, and A_{71c}⁻ in formula (IIa-2) are each the same as that of A₁₁⁻ in formula (Ia-1) above, and their preferred modes are also the same as that of A₁₁⁻. The definitions of M₇₁ₐ⁺, M_{71b}⁺, and M_{71c}⁺ are each the same as that of M₁₁⁺ in formula (Ia-1), and their preferred modes are also the same as that of M₁₁⁺.

The definitions of L₇₁, L₇₂, and L₇₃ in formula (IIa-2) are each the same as that of L₁ in formula (Ia-1) above, and their preferred modes are also the same as that of L₁.

In a compound PIIa-2 formed by replacing each of the organic cations represented by M₇₁ₐ⁺, M_{71b}⁺, and M_{71c}⁺ in formula (IIa-2) with H⁺, the acid dissociation constant a1-9 derived from an acidic moiety represented by A₇₁ₐH, the acid dissociation constant a1-10 derived from an acidic moiety represented by A_{71b}H, and the acid dissociation constant a1-11 derived from an acidic moiety represented by A_{71c}H each correspond to the acid dissociation constant a1 described above.

The compound PIIa-2 formed by replacing each of the cationic moieties M₇₁ₐ⁺, M_{71b}⁺, and M_{71c}⁺ in the structural moieties X in the formula (IIa-1) with H⁺ corresponds to HA₇₁ₐ-L₇₁-N(L₇₃-A_{71c}H)-L₇₂-A₇₁H. The compound PIIa-2 is the same as the acid generated from the compound represented by formula (IIa-2) upon irradiation with actinic rays or radiation.

At least one of M₇₁ₐ⁺, M_{71b}⁺, M_{71c}⁺, A₇₁ₐ⁻, A_{71b}⁻, A_{71c}⁻, L₇₁, L₇₂, or L₇₃ may have an acid-decomposable group as a substituent.

Examples of the anionic moiety that the compound (I) and the compound (II) can have are shown below, but the invention is not limited thereto.

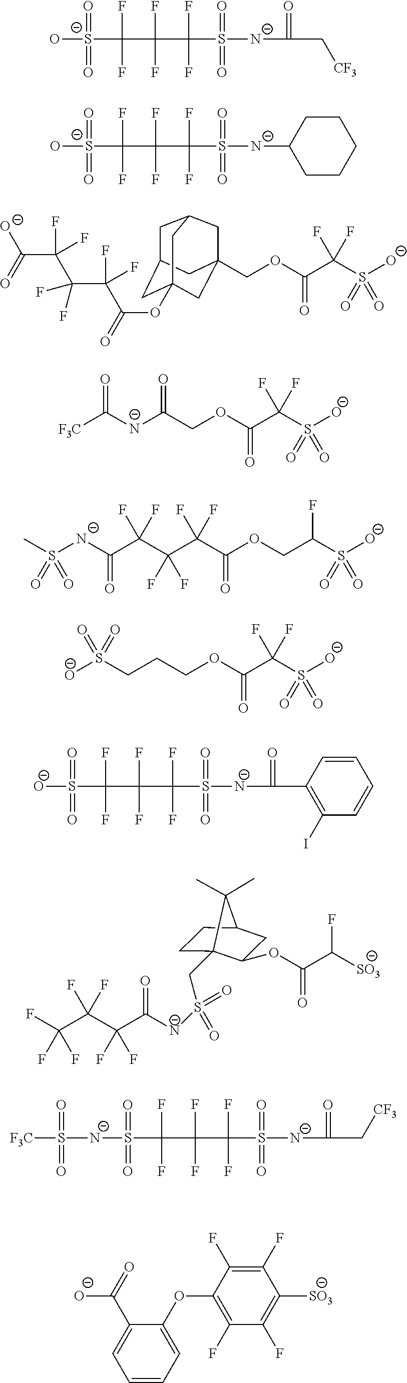

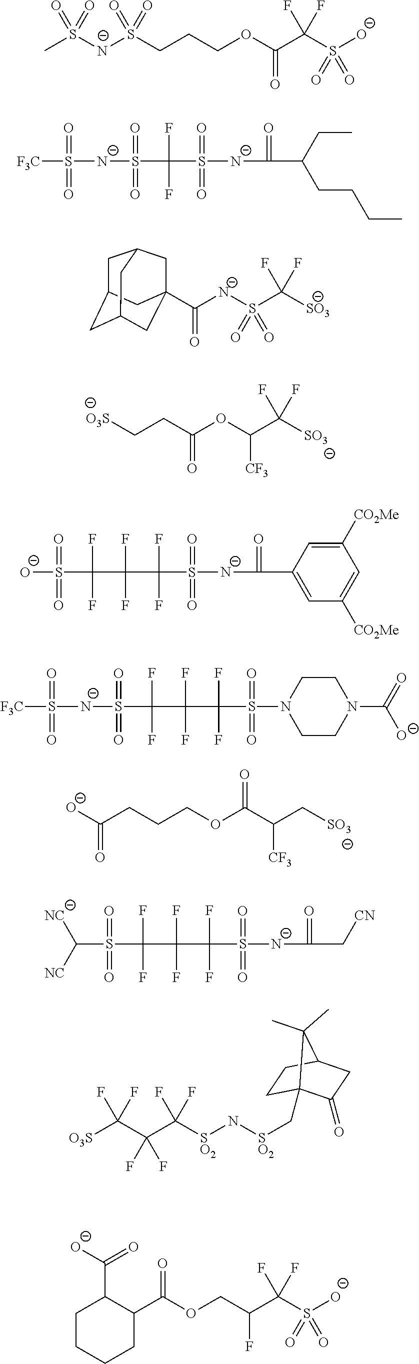

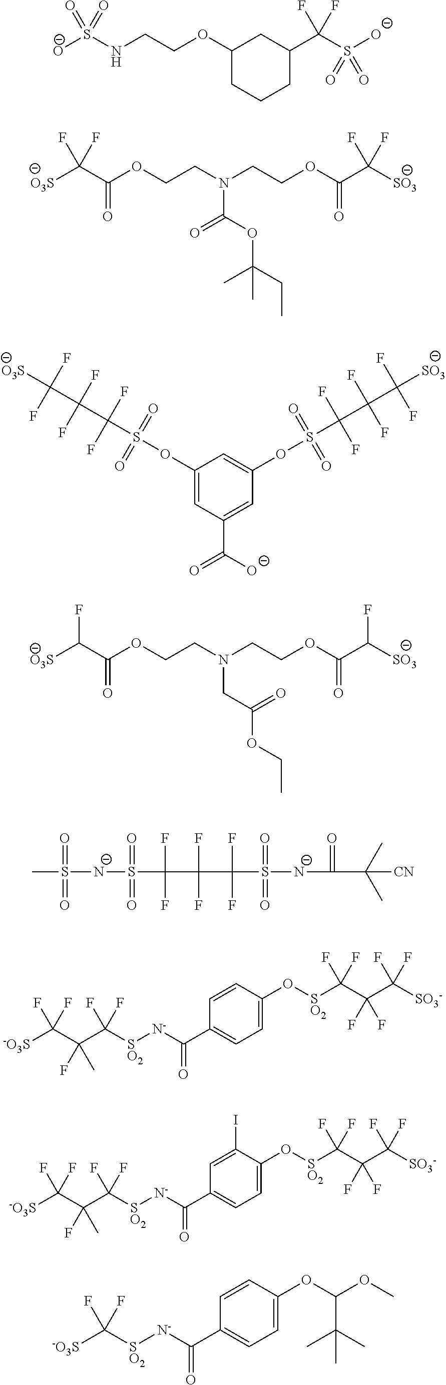

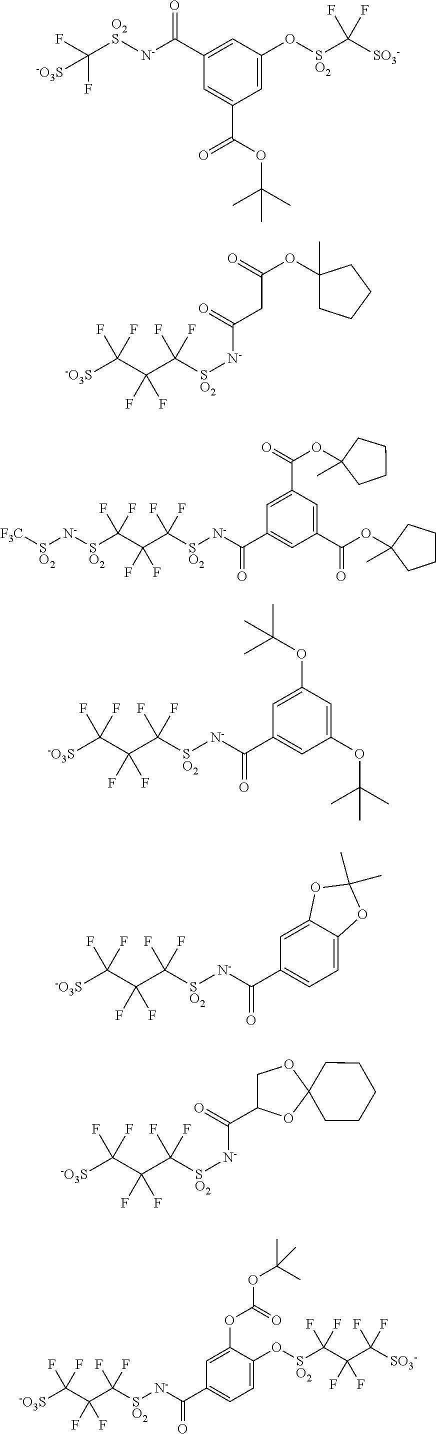

The compound (B) used is, for example, preferably any of photoacid generators disclosed in paragraphs [0135] to [0171] of WO2018/193954A, paragraphs [0077] to [0116] of WO2020/066824A, and paragraphs [0018] to [0075] and [0334] to [0335] of WO2017/154345A.

No particular limitation is imposed on the content of the compound (B) in the composition of the invention. The content of the compound (B) with respect to the total amount of the solids in the composition of the invention is preferably 0.1% by mass or more, more preferably 1% by mass or more, and still more preferably 5% by mass or more. The content of the compound (B) with respect to the total amount of the solids in the composition of the invention is preferably 60% by mass or less, more preferably 50% by mass or less, and still more preferably 40% by mass or less.

One compound (B) may be used alone, or two or more compounds (B) may be used.

### [Acid Diffusion Control Agent]

Preferably, the composition of the invention includes an acid diffusion control agent. The acid diffusion control agent functions as a quencher that traps the acid generated from the photoacid generator etc. during exposure to light to thereby suppress the reaction of the resin (A) with an excess portion of the generated acid in unexposed portions.

Examples of the acid diffusion control agent that can be used include a basic compound (DA), a basic compound (DB) whose basicity decreases or disappears upon irradiation with actinic rays or radiation, an onium salt (DC) that generates an acid weaker than the acid generated from the photoacid generator (B), a low-molecular weight compound (DD) having a nitrogen atom and having a group that leaves by the action of an acid, and an onium salt compound (CD) having a nitrogen atom in its cationic moiety. In the composition of the invention, any well-known acid diffusion control agent can be appropriately used. For example, any of well-known compounds disclosed in paragraphs [0627] to [0664] of US2016/0070167A, paragraphs [0095] to [0187] of US2015/0004544A, paragraphs [0403] to [0423] of US2016/0237190A, and paragraphs [0259] to [0328] of US2016/0274458A can be preferably used as the acid diffusion control agent.

The basic compound (DA) is preferably a compound having a structure represented by any of the following general formulas (A) to (E).

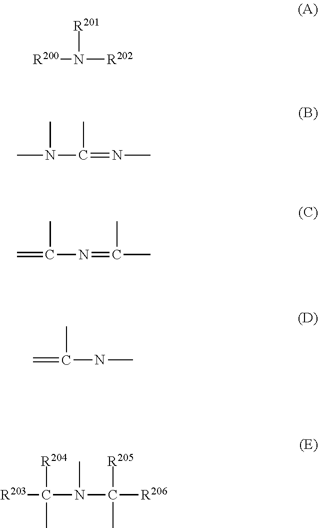

In general formulas (A) and (E),

R²⁰⁰, R²⁰¹, and R²⁰² may be the same or different and each independently represent a hydrogen atom, an alkyl group (having preferably 1 to 20 carbon atoms), a cycloalkyl group (having preferably 3 to 20 carbon atoms), or an aryl group (having 6 to 20 carbon atoms). R²⁰¹ and R²⁰² may be bonded together to form a ring.

R²⁰³, R²⁰4, R²⁰⁵, and R²⁰⁶ may be the same or different and each independently represent an alkyl group having 1 to 20 carbon atoms.

In general formulas (A) and (E), the alkyl group may have a substituent or may be unsubstituted.

As for the alkyl group, the alkyl group having a substituent is preferably an aminoalkyl group having 1 to 20 carbon atoms, a hydroxyalkyl group having 1 to 20 carbon atoms, or a cyanoalkyl group having 1 to 20 carbon atoms.

In general formulas (A) and (E), the alkyl group is preferably unsubstituted.

The basic compound (DA) is preferably thiazole, benzothiazole, oxazole, benzoxazole, guanidine, aminopyrrolidine, pyrazole, pyrazoline, piperazine, aminomorpholine, aminoalkylmorpholine, piperidine, or a compound having any of the above structures and more preferably a compound having a thiazole structure, a benzothiazole structure, an oxazole structure, a benzoxazole structure, an imidazole structure, a diazabicyclo structure, an onium hydroxide structure, an onium carboxylate structure, a trialkylamine structure, an aniline structure, or a pyridine structure, an alkylamine derivative having a hydroxy group and/or an ether bond, or an aniline derivative having a hydroxy group and/or an ether bond.

The basic compound (DB) whose basicity decreases or disappears upon irradiation with actinic rays or radiation (this compound is referred to also as the “compound (DB)”) is a compound that has a proton-accepting functional group and is decomposed upon irradiation with actinic rays or radiation to cause its proton acceptability to decrease or disappear or to undergo a change from the proton-accepting compound to an acidic compound.

The proton-accepting functional group is a functional group having a group or electron capable of electrostatically interacting with a proton and means, for example, a functional group having a macrocyclic structure such as cyclic polyether or a functional group having a nitrogen atom having an unshared electron pair not contributing to π conjugation. The nitrogen atom having an unshared electron pair not contributing to π conjugation is, for example, a nitrogen atom having a partial structure represented by any of the following formulas.

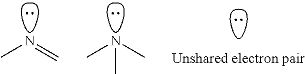

Preferred examples of the partial structure of the proton-accepting functional group include crown ether structures, azacrown ether structures, primary to tertiary amine structures, pyridine structures, imidazole structures, and pyrazine structures.

The compound (DB) is decomposed upon irradiation with actinic rays or radiation to cause its proton acceptability to decrease or disappear or to undergo a change from the proton-accepting compound to an acidic compound. The reduction or disappearance of the proton acceptability or the change from the proton-accepting compound to the acidic compound means the change in the proton acceptability caused by addition of a proton to the proton-accepting functional group and specifically means a reduction in the equilibrium constant of the chemical equilibrium when a proton adduct is formed from the compound (DB) having the proton-accepting functional group and a proton.

The proton acceptability can be checked by performing pH measurement.

The acid dissociation constant pKa of the compound generated when the compound (DB) is decomposed upon irradiation with actinic rays or radiation satisfies preferably pKa<−1, satisfies more preferably −13<pKa<−1, and satisfies still more preferably −13<pKa<−3.

A mixture of the photoacid generator (B) and the onium salt (DC) that generates an acid weaker than the acid generated from the photoacid generator (B) may be used. In this case, when the acid generated from the photoacid generator(B) upon irradiation with actinic rays or radiation collides with the onium salt (DC) having an unreacted weak acid anion, a weak acid is released by salt exchange, and an onium salt having a strong acid anion is thereby generated. In this process, the strong acid is converted to the weak acid having a lower catalytic ability, and the acid is apparently inactivated, so that the diffusion of the acid can be controlled.

The onium salt (DC) is preferably a compound represented by any of the following general formulas (d1-1) to (d1-3).

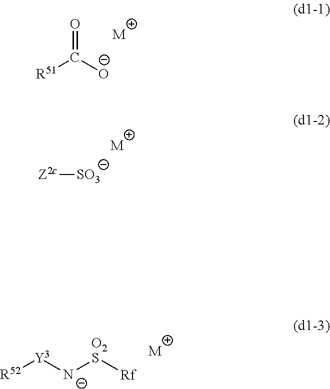

In these formulas, R⁵¹ is a hydrocarbon group optionally having a substituent, and Z^{2c} is a hydrocarbon group having 1 to 30 carbon atoms and optionally having a substituent (provided that carbon adjacent to S is not substituted with a fluorine atom). R⁵² is an organic group, and Y³ is a linear, branched, or cyclic alkylene group or an arylene group. Rf is a hydrocarbon group including a fluorine atom, and M⁺'s each independently represent an ammonium cation, a sulfonium cation, or an iodonium cation.

Preferred examples of the sulfonium and iodonium cations represented by M⁺ include those for the sulfonium cation represented by general formula (ZI) and those for the iodonium cation represented by general formula (ZII).

The onium salt (DC) that generates an acid weaker than the photoacid generator may be a compound having a cationic moiety and an anionic moiety in one molecule with the cationic moiety and the anionic moiety linked via covalent bonding (this compound is hereinafter referred to as a “compound (DCA)”).

The compound (DCA) is preferably a compound represented by any of the following general formulas (C-1) to (C-3).

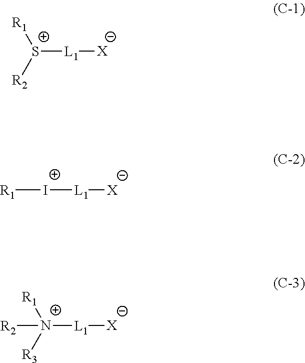

In general formulas (C-1) to (C-3),

R₁, R₂, and R₃ each independently represent a substituent having at least one carbon atom.

L₁ represents a divalent linking group or a single bond linking the cationic moiety and the anionic moiety.

—X⁻ represents an anionic moiety selected from —COO—, —SO₃⁻, —SO₂⁻, and —N—R₄. R₄ represents a monovalent substituent having at least one selected from a carbonyl group (—C(═O)—), a sulfonyl group (—S(═O)₂—), and a sulfinyl group (—S(═O)—) at a site linked to the adjacent N atom.

R₁, R₂, R₃, R₄, and L₁ may be bonded together to form a ring structure. In general formula (C-3), two selected from the group consisting of R₁ to R₃ may together represent one divalent substituent and may be bonded to the N atom via a double bond.

Examples of the substituent having at least one carbon atom and represented by any of R₁ to R₃ include alkyl groups, cycloalkyl groups, aryl groups, alkyloxycarbonyl groups, cycloalkyloxycarbonyl groups, aryloxycarbonyl groups, alkylaminocarbonyl groups, cycloalkylaminocarbonyl groups, and arylaminocarbonyl groups. The substituent is preferably an alkyl group, a cycloalkyl group, or an aryl group.

Examples of L₁ serving as a divalent linking group include linear and branched alkylene groups, cycloalkylene groups, arylene groups, a carbonyl group, an ether bond, an ester bond, an amide bond, a urethane bond, a urea bond, and groups formed by combining any two or more of them. L₁ is preferably an alkylene group, an arylene group, an ether bond, an ester bond, or a group formed by combining any two or more of them.

The low-molecular weight compound (DD) having a nitrogen atom and having a group that leaves by the action of an acid (this compound is hereinafter referred to also as the “compound (DD)” is preferably an amine derivative having, on the nitrogen atom, the group that leaves by the action of an acid.

The group that leaves by the action of an acid is preferably an acetal group, a carbonate group, a carbamate group, a tertiary ester group, a tertiary hydroxy group, or a hemiaminal ether group and more preferably a carbamate group or a hemiaminal ether group.

The molecular weight of the compound (DD) is preferably 100 to 1000, more preferably 100 to 700, and still more preferably 100 to 500.

The compound (DD) may have a carbamate group having a protecting group on the nitrogen atom. The protecting group included in the carbamate group is represented by the following general formula (d-1).

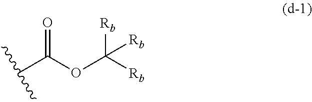

In general formula (d-1),

R_{b}'s each independently represent a hydrogen atom, an alkyl group (having preferably 1 to 10 carbon atoms), a cycloalkyl group (having preferably 3 to 30 carbon atoms), an aryl group (having preferably 3 to 30 carbon atoms), an aralkyl group (having preferably 1 to 10 carbon atoms), or an alkoxyalkyl group (having preferably 1 to 10 carbon atoms). R_{b}'s may be bonded together to form a ring.

The alkyl, cycloalkyl, aryl, and aralkyl groups represented by R_{b}'s may be each independently substituted with a functional group such as a hydroxyl group, a cyano group, an amino group, a pyrrolidino group, a piperidino group, a morpholino group, or an oxo group, an alkoxy group, or a halogen atom.

The same applies to the alkoxyalkyl group represented by each R_{b}.

Each R_{b} is preferably a linear or branched alkyl group, a cycloalkyl group, or an aryl group and more preferably a linear or branched alkyl group or a cycloalkyl group.

Examples of the ring formed by linking two R_{b}'s together include aliphatic hydrocarbons, aromatic hydrocarbons, heterocyclic hydrocarbons, and derivatives thereof.

Specific examples of structure of the group represented by general formula (d-1) include structures disclosed in paragraph [0466] of US2012/0135348A, but the group represented by general formula (d-1) is not limited thereto.

Preferably, the compound (DD) has a structure represented by the following general formula (6).

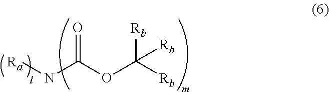

In general formula (6),

l represents an integer of 0 to 2, and m represents an integer of 1 to 3. l+m=3 is satisfied.

Rₐ represents a hydrogen atom, an alkyl group, a cycloalkyl group, an aryl group, or an aralkyl group. When 1 is 2, two Rₐ's may be the same different. The two Rₐ's may be linked together to form a heterocycle together with the nitrogen atom in the formula. The heterocycle may include a heteroatom other than the nitrogen atom in the formula.

R_{b}'s have the same meaning as R_{b}'s in general formula (d-1), and their preferred examples are also the same as those of R_{b}'s in general formula (d-1).

In general formula (6), the alkyl, cycloalkyl, aryl, and aralkyl groups represented by Rₐ may be each independently substituted with any of the groups described above as the groups with which the alkyl, cycloalkyl, aryl, and aralkyl groups represented by R_{b} can be substituted.

Specific examples of the alkyl, cycloalkyl, aryl, and aralkyl groups represented by Rₐ(these groups may be substituted with any of the groups described above) include those described as the specific examples of R_{b}.

Particularly preferable specific examples of the compound (DD) in the invention include compounds disclosed in paragraph [0475] of US2012/0135348A, but the compound (DD) is not limited thereto.

The onium salt compound (DE) having a nitrogen atom in its cationic moiety (this compound is hereinafter referred to also as the “compound (DE)”) is preferably a compound having a basic moiety including a nitrogen atom in the cationic moiety. The basic moiety is preferably an amino group and more preferably an aliphatic amino group. Still more preferably, all the atoms adjacent to the nitrogen atom in the basic moiety are each a hydrogen atom or a carbon atom. From the viewpoint of improving the basicity, it is preferable that an electron-withdrawing functional group (such as a carbonyl group, a sulfonyl group, a cyano group, or a halogen atom) is not bonded directly to the nitrogen atom.

Specific examples of the compound (DE) include compounds disclosed in paragraph [203] of US2015/0309408A, but the compound (DE) is not limited thereto.

As for specific examples of the acid diffusion control agent, reference can be made to the description in paragraphs [0204] to [0206] of WO2018/193954A, the contents of which are incorporated herein. However, the acid diffusion control agent that can be used in the invention is not limited thereto.

One acid diffusion control agent may be used alone, or two or more acid diffusion control agents may be used in combination.

When the composition of the invention includes the acid diffusion control agent, the content of the acid diffusion control agent (the total content when a plurality of acid diffusion control agents are present) in the composition of the invention with respect to the total amount of the solids in the composition of the invention is preferably 0.001 to 20% by mass and more preferably 0.01 to 15% by mass.

### [Solvent]

Preferably, the composition of the invention includes a solvent.

Any known resist solvent may be appropriately used as the solvent for the composition of the invention.

Examples of the solvent include organic solvents such as alkylene glycol monoalkyl ether carboxylates, alkylene glycol monoalkyl ethers, lactic acid alkyl esters, alkyl alkoxypropionates, cyclic lactones (having preferably 4 to 10 carbon atoms), monoketone compounds (having preferably 4 to 10 carbon atoms) optionally having a ring, alkylene carbonates, alkyl alkoxyacetates, and alkyl pyruvates.

As for the solvent, reference can be made to the description in paragraphs [0187] to [0197] of WO2019/058890A, the contents of which are incorporated herein.

The concentration of the solids in the actinic ray-sensitive or radiation-sensitive resin composition of the invention is generally 1.0 to 30% by mass and preferably 1.5 to 10% by mass. When the concentration of the solids is within the above range, the resist solution can be applied uniformly to a substrate.

The concentration of the solids is the mass percentage of components other than the solvent with respect to the total mass of the actinic ray-sensitive or radiation-sensitive resin composition.

### [Surfactant]

The composition of the invention may further include a surfactant. The surfactant included allows a pattern with less adhesiveness and fewer development defects to be formed with high sensitivity and resolution when an exposure light source with a wavelength of 250 nm or less, in particular 220 nm or less, is used.

Particularly preferably, the surfactant used is a fluorine-based and/or silicon-based surfactant.

As for the surfactant, reference can be made to the description in paragraphs [0183] to [0184] of WO2019/058890A, the contents of which are incorporated herein.

When the composition of the invention includes the surfactant, the content of the surfactant with respect to the total mass of the solids in the composition is preferably 0 to 2% by mass, more preferably 0.0001 to 2% by mass, and still more preferably 0.0005 to 1% by mass.

### [Additional Additives]

The composition of the invention may further appropriately include, in addition to the components described above, a carboxylic acid, a carboxylic acid onium salt, a dissolution inhibiting compound having a molecular weight of 3000 or less and described in, for example, Proceeding of SPIE, 2724, 355 (1996), a dye, a plasticizer, a photosensitizer, a light absorber, an antioxidant, etc.

In particular, the carboxylic acid is preferably used to improve performance. The carboxylic acid is preferably an aromatic carboxylic acid such as benzoic acid or naphthoic acid.

When the composition of the invention includes the carboxylic acid, the content of the carboxylic acid with respect to the total mass of the solids in the composition is preferably 0.01 to 10% by mass, more preferably 0.01 to 5% by mass, and still more preferably 0.01 to 3% by mass.

### [Applications]

The composition of the invention relates to an actinic ray-sensitive or radiation-sensitive resin composition that undergoes a reaction upon irradiation with actinic rays or radiation and changes its properties. More specifically, the composition of the invention relates to an actinic ray-sensitive or radiation-sensitive resin composition that is used for processes for manufacturing semiconductors such as ICs (Integrated Circuits), manufacturing of circuit boards for liquid crystals, thermal heads, etc., production of imprint mold structures, other photofabrication processes, lithographic printing plates, and manufacturing of acid-curable compositions. The pattern formed in the invention can be used for etching processes, ion implantation processes, bump electrode forming processes, rewiring forming processes, MEMS (Micro Electro Mechanical Systems), etc.

### [Actinic Ray-Sensitive or Radiation-Sensitive Film]

The present invention also relates to an actinic ray-sensitive or radiation-sensitive film (preferably a resist film) formed using the above-described actinic ray-sensitive or radiation-sensitive resin composition of the invention. This film is formed, for example, by applying the composition of the invention to a support such as a substrate. No particular limitation is imposed on the thickness of the actinic ray-sensitive or radiation-sensitive film, but the thickness is preferably 0.02 to 0.1 m. The composition is applied to the substrate using an appropriate coating method such as spin coating, roll coating, flow coating, dip coating, spray coating, or doctor coating, and spin coating is preferred. The rotation speed is preferably 1000 to 3000 rpm (rotations per minute). The applied film is pre-baked at 60 to 150° C. for 1 to 20 minutes, preferably at 80 to 120° C. for 1 to 10 minutes, to form a thin film.

As for the substrate and a topcoat that may be formed on the actinic ray-sensitive or radiation-sensitive film, reference can be made to paragraphs [0342] to [0358] of WO2017/056832A, the contents of which are incorporated herein.

### [Pattern Forming Method]

The present invention also relates to a pattern forming method including:
a resist film forming step of forming a resist film using the actinic ray-sensitive or radiation-sensitive resin composition of the invention;
an exposure step of exposing the resist film to light; and
a development step of developing the exposed resist film using a developer.

In the present invention, the exposure to light is performed using preferably an electron beam (EB), an ArF excimer laser, or extreme ultraviolet rays (EUV) and using more preferably an electron beam or extreme ultraviolet rays.

In the manufacturing of precision integrated circuit elements etc., it is preferable that the resist film of the invention is exposed to light by irradiating the resist film with an ArF excimer laser beam, an electron beam, or extreme ultraviolet rays (EUV) through a pattern (a pattern forming step). The light exposure is performed such that the exposure amount is about 1 to about 100 mJ/cm² and preferably about 20 to about 60 mJ/cm² for the ArF excimer laser beam, about 0.1 to about 20 μC/cm² and preferably about 3 to about 10 μC/cm 2 for the electron beam, and about 0.1 to about 20 mJ/cm² and preferably about 3 to about 15 mJ/cm² for the extreme ultraviolet rays.

Next, the resulting resist film is post-exposure baked on a hot plate preferably at 60 to 150° C. for 5 seconds to 20 minutes, more preferably at 80 to 120° C. for 15 seconds to 10 minutes, and still more preferably at 80 to 120° C. for 1 to 10 minutes, then developed, rinsed, and dried to form a pattern. The post-exposure baking is appropriately controlled according to the acid decomposability of the repeating unit having the acid-decomposable group in the resin (A). When the acid decomposability is low, the temperature in the post-exposure baking is also preferably 110° C. or higher, and the heating time is preferably 45 seconds or longer.

An appropriate developer is selected, and it is preferable to use an alkali developer (typically an aqueous alkali solution) or a developer including an organic solvent (which may be referred to also as an organic-based developer). When the developer is an aqueous alkali solution, the resist film is developed using a 0.1 to 5% by mass aqueous alkali solution, preferably a 2 to 3% by mass aqueous alkali solution, of tetramethylammonium hydroxide (TMAH), tetrabutylammonium hydroxide (TBAH), etc. for 0.1 to 3 minutes, preferably 0.5 to 2 minutes, by a routine method such as a dipping method, a puddle method, or a spray method.

An appropriate amount of alcohol and/or an appropriate amount of a surfactant may be added to the alkali developer. In the case of the formation of a negative-type pattern, the film in the unexposed portions is dissolved in the developer, and the film in the exposed portions is not easily dissolved, so that an intended pattern is formed on the substrate. In the case of the formation of a positive-type pattern, the film in the exposed portions is dissolved in the developer, and the film in the unexposed portions is not easily dissolved, so that an intended pattern is formed on the substrate.

The alkali concentration of the alkali developer is generally 0.1 to 20% by mass.

The pH of the alkali developer is generally 10.0 to 15.0.

In particular, the alkali developer is preferably a 2.38% by mass aqueous tetramethylammonium hydroxide solution.

A rinsing solution used in the rinsing treatment performed after the alkali development may be pure water, and an appropriate amount of a surfactant may be added to the rinsing solution used.

After the development treatment or the rinsing treatment, treatment for removing the developer or the rinsing solution adhering to the pattern using a supercritical fluid may be performed.

When the pattern forming method of the invention includes the step of developing using a developer including an organic solvent, the developer used in this step (hereinafter referred to also as an organic-based developer) may be a polar solvent such as a ketone-based solvent, an ester-based solvent, an alcohol-based solvent, an amide-based solvent, or an ether-based solvent or a hydrocarbon-based solvent.

The concentration of the organic solvent in the organic-based developer (the total concentration when a mixture of a plurality of organic solvents is used) is preferably 50% by mass or more, more preferably 50 to 100% by mass, still more preferably 85 to 100% by mass, yet more preferably 90 to 100% by mass, and particularly preferably 95 to 100% by mass.

It is most preferable that the organic-based developer consists essentially only of the organic solvent. The phrase “consists essentially only of the organic solvent” is intended to encompass the case in which the organic-based developer includes trace amounts of a surfactant, an antioxidant, a stabilizer, an anti-foaming agent, etc.

In particular, the organic-based developer is preferably a developer including at least one organic solvent selected from the group consisting of a ketone-based solvent, an ester-based solvent, an alcohol-based solvent, an amide-based solvent, and an ether-based solvent.

As for the pattern forming method, reference can be made to the description in paragraphs [0359] to [0383] of WO2017/056832A, the contents of which are incorporated herein.

Preferably, the actinic ray-sensitive or radiation-sensitive resin composition and various materials used for the pattern forming method of the invention (such as the resist solvent, the developer, the rinsing solution, a composition for forming an antireflection film, and a composition for forming the topcoat) include no impurities such as metals, halogen-containing metal salts, acids, alkalis, and components including sulfur atoms or phosphorus atoms. Examples of the impurities including metal atoms include Na, K, Ca, Fe, Cu, Mn, Mg, Al, Cr, Ni, Zn, Ag, Sn, Pb, Li, and salts of these metals.

The content of the impurities included in each of these materials is preferably 1 ppm (parts per million) or less, more preferably 1 ppb (parts per billion) or less, still more preferably 100 ppt (parts per trillion) or less, and particularly preferably 10 ppt or less, and it is most preferable that these materials include substantially no impurities (equal to or less than the detection limit of the measurement device).

As for the method for removing impurities such as metals from these materials, reference can be made to the description in paragraphs [0384] to [0402] of WO2017/056832A, the contents of which are incorporated herein.

### [Method for Manufacturing Electronic Device]

The invention also relates to an electronic device manufacturing method including the pattern forming method described above. The electronic device manufactured by the electronic device manufacturing method of the invention is preferably installed in electric and electronic devices (such as household electrical appliances, OA (Office Automation) related devices, media related devices, optical devices, and communication devices).

### EXAMPLES

The present invention will be further described in detail by way of Examples. Materials, amounts used, ratios, treatment details, treatment procedures, etc. shown in the following Examples can be appropriately changed so long as they do not depart from the gist of the invention. Therefore, the scope of the present invention should not be construed as limited to the following Examples.

### <Resins (A)>

The structures of repeating units of resins (A) used, their contents (molar ratios), their weight average molecular weights (Mw), and their dispersities (Pd=Mw/Mn) are shown below. Although a resin (AX-1) shown below does not have any of the repeating unit represented by general formula (3), the repeating unit represented by general formula (6), and the repeating unit represented by general formula (7) and is not the resin (A), the resin (AX-1) is placed in the resin (A) column for the sake of convenience.

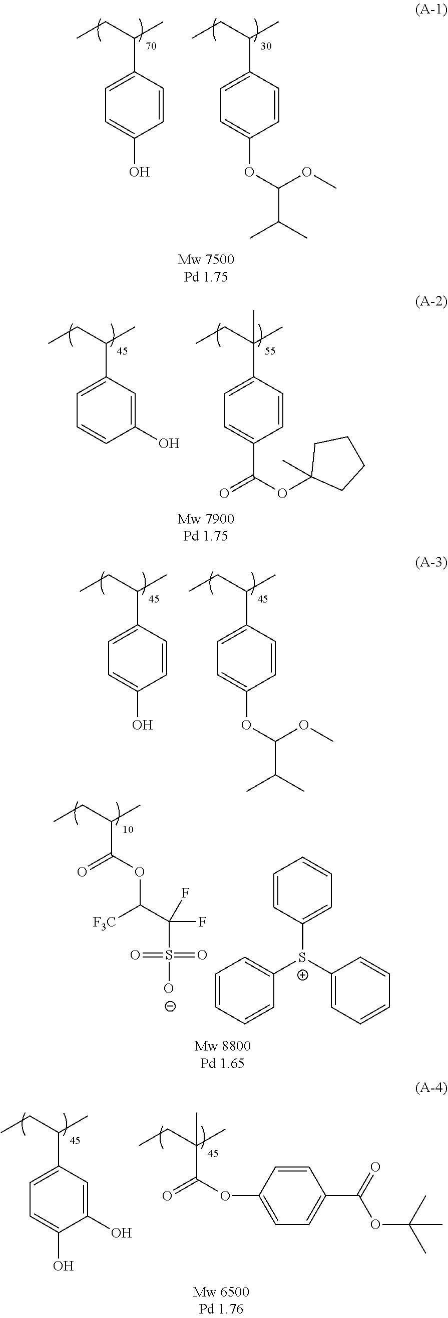

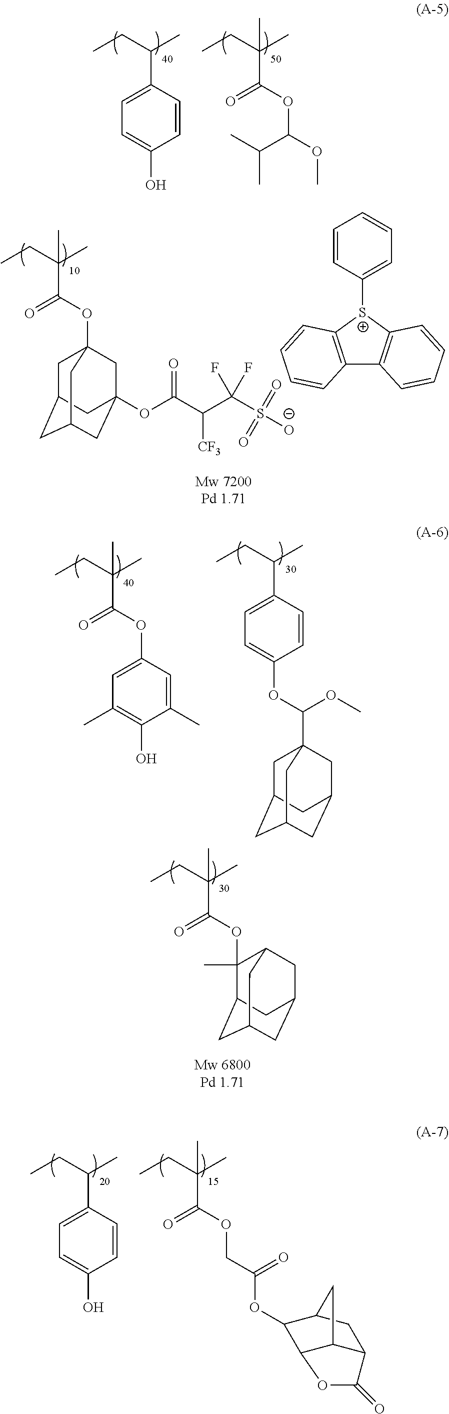

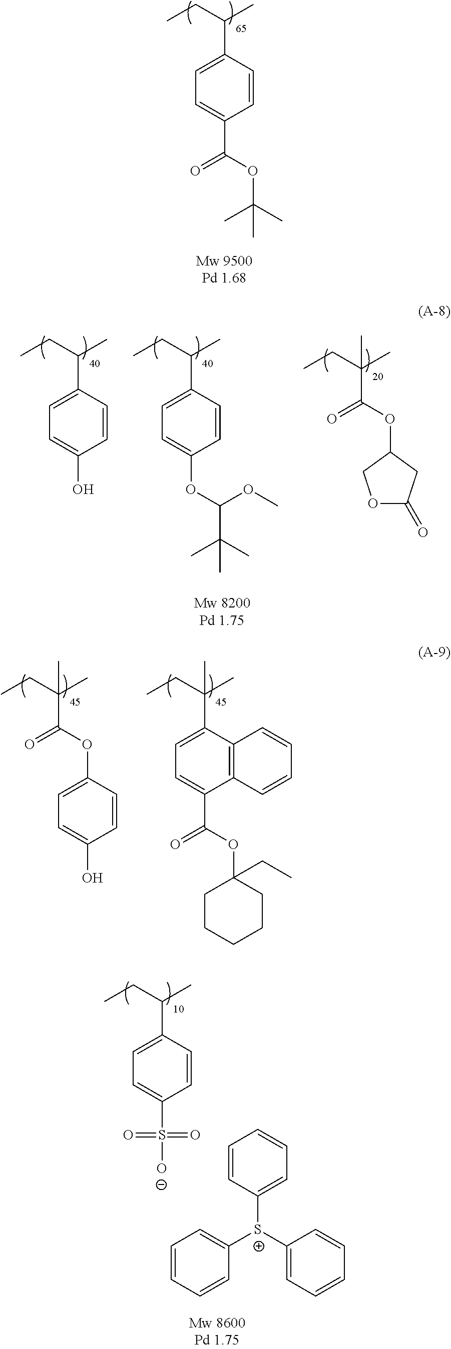

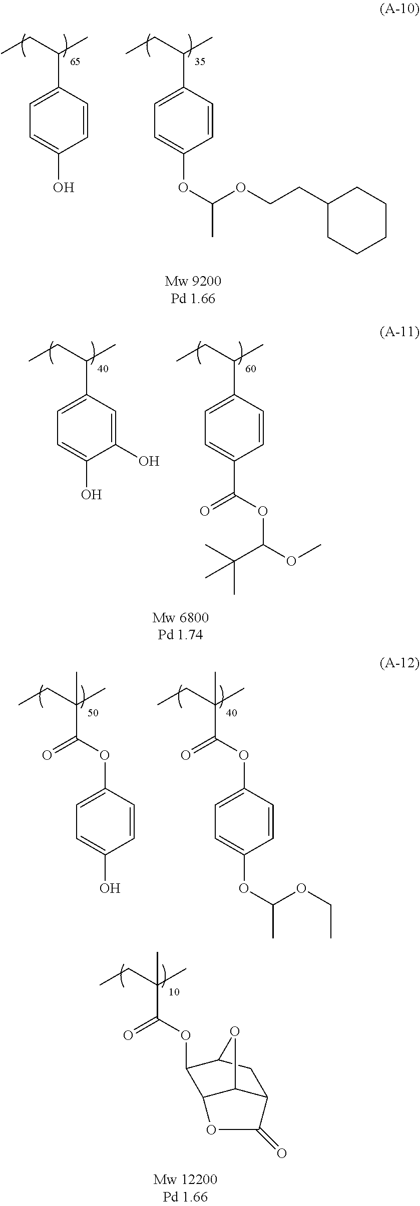

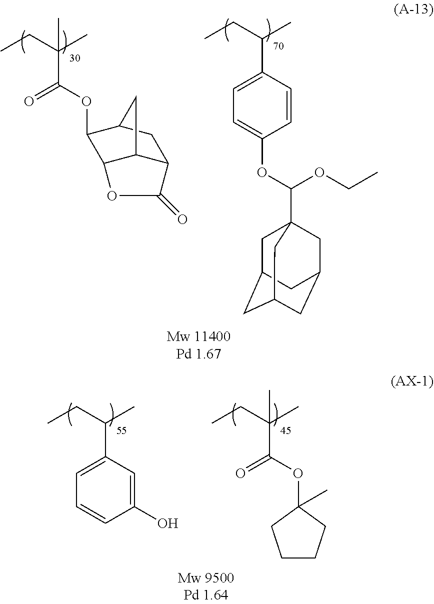

### Synthesis Example 1: Synthesis of Resin (A-1)

Cyclohexanone (57 g) was heated to 85° C. in a nitrogen flow. A solution mixture of a monomer (50.5 g) represented by formula (M-1) below, a monomer (37.1 g) represented by formula (M-2) below, cyclohexanone (106 g), and 2,2′-azobisisobutyric acid dimethyl ester [V-601 manufactured by FUJIFILM Wako Pure Chemical Corporation] (8.6 g) was added dropwise to the solution over 3 hours under stirring to thereby obtain a reaction solution. After completion of the dropwise addition, the reaction solution was further stirred at 85° C. for 3 hours. The resulting reaction solution was allowed to cool, then subjected to reprecipitation using 4100 g of ethyl acetate/heptane (mass ratio 1:9), and filtrated, and the solid obtained was vacuum-dried to thereby obtain a resin (A-1) (86 g).

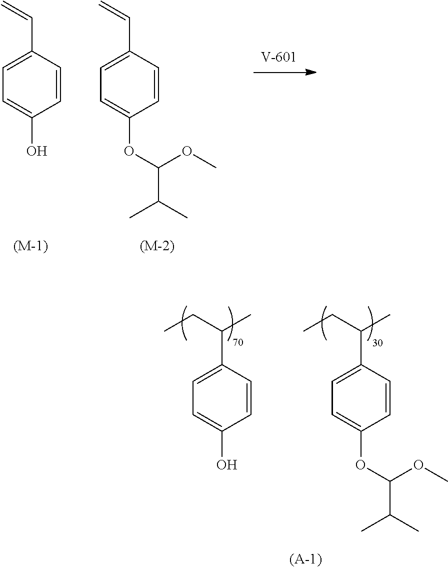

The other resins were synthesized in the same manner as above.

### <Photoacid Generators (B)>

The structures of photoacid generators (B) used are shown below.

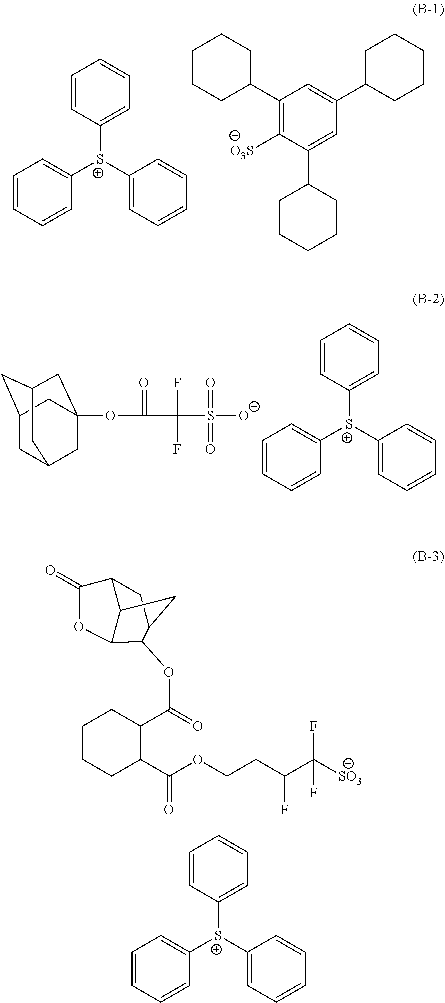

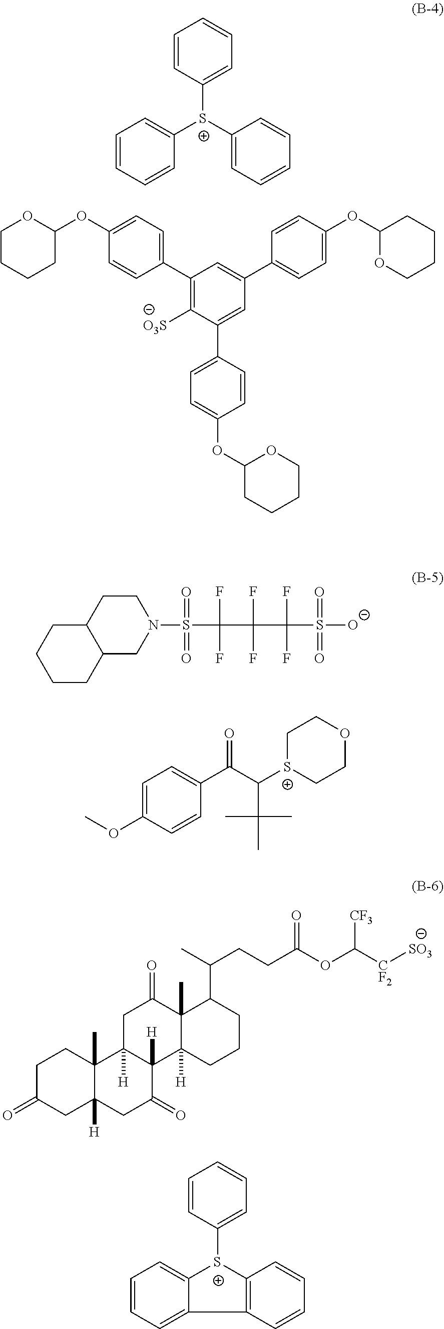

### <Acid Diffusion Control Agents>

The structures of acid diffusion control agents used are shown below.

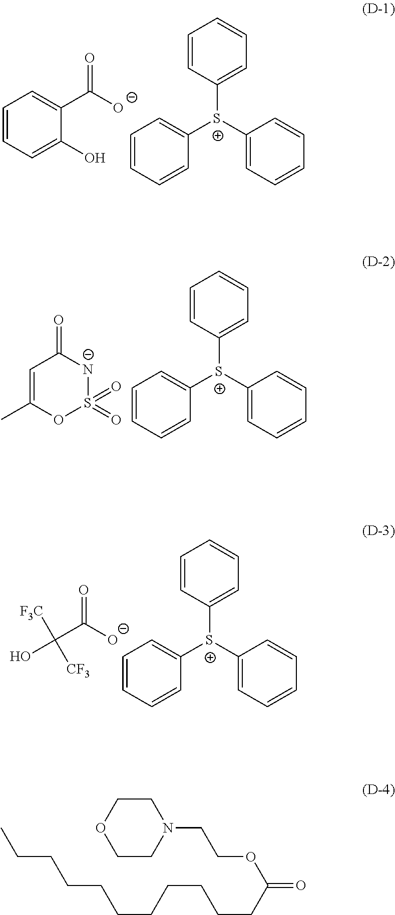

### <Acidic Compounds (F)>

The structures of acidic compounds (F) used are shown below. Although a compound (FX-1) below has no iodine atom and is not the acidic compound (F), the compound (FX-1) is placed in the acidic compound (F) column for the sake of convenience.

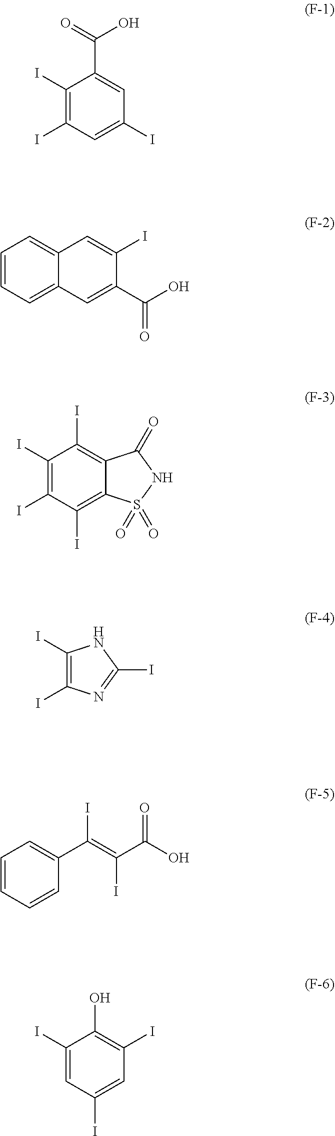 [SMILES: O=C(O)c1cc(I)cc(I)c1I ; O=C(O)c1cc2ccccc2cc1I ; O=C1NS(=O)(=O)c2c(I)c(I)c(I)c(I)c21 ; Ic1nc(I)c(I)[nH]1 ; O=C(O)/C(I)=C(\I)c1ccccc1 ; Oc1c(I)cc(I)cc1I]

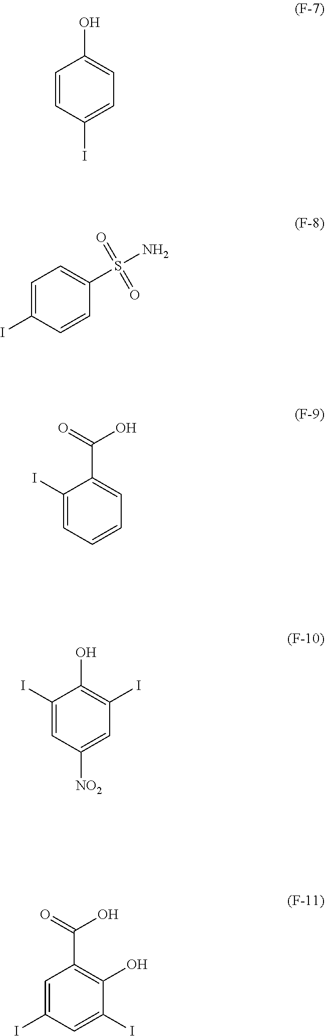 [SMILES: Oc1ccc(I)cc1 ; NS(=O)(=O)c1ccc(I)cc1 ; O=C(O)c1ccccc1I ; O=[N+]([O-])c1cc(I)c(O)c(I)c1 ; O=C(O)c1cc(I)cc(I)c1O]

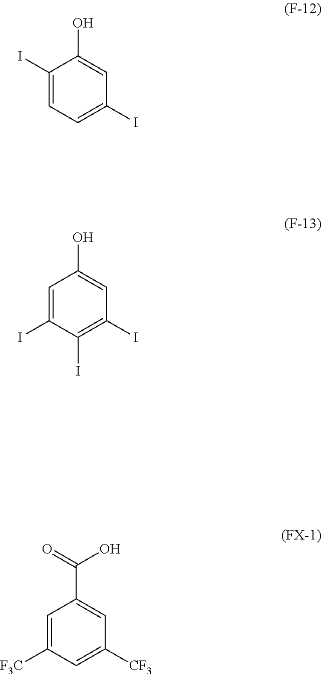 [SMILES: Oc1cc(I)ccc1I ; Oc1cc(I)c(I)c(I)c1 ; O=C(O)c1cc(C(F)(F)F)cc(C(F)(F)F)c1]

The pKa of each acidic compound (F) is shown in Table 1 below.

TABLE 1

| Acidic compound (F) | pKa |
|---|---|
| (F-1) | 2.16 |
| (F-2) | 2.86 |
| (F-3) | 0.41 |
| (F-4) | 7.47 |
| (F-5) | 1.51 |
| (F-6) | 6.47 |
| (F-7) | 9.30 |
| (F-8) | 9.88 |
| (F-9) | 2.86 |
| (F-10) | 3.85 |
| (F-11) | 2.07 |
| (F-12) | 7.66 |
| (F-13) | 7.67 |
| (FX-1) | 3.34 |

### <Surfactant>

The following surfactants were used.

W-1: MEGAFACE F176 (manufactured by DIC Corporation; fluorine-based surfactant)

W-2: MEGAFACE R08 (manufactured by DIC Corporation; fluorine and silicon-based surfactant)

W-3: Polysiloxane polymer KP-341 (manufactured by Shin-Etsu Chemical Co., Ltd.; silicon based surfactant)

W-4: Troysol S-366 (manufactured by Troy Chemical)

W-5: KH-20 (manufactured by Asahi Glass Co., Ltd.)

W-6: PolyFox PF-6320 (manufactured by OMNOVA Solutions Inc.; fluorine-based surfactant)

### <Solvents>

The following solvents were used.

SL-1: Propylene glycol monomethyl ether acetate (PGMEA)

SL-2: Propylene glycol monomethyl ether propionate

SL-3: 2-Heptanone

SL-4: Ethyl lactate

SL-5: Propylene glycol monomethyl ether (PGME)

SL-6: Cyclohexanone

SL-7: 7-Butyrolactone

SL-8: Propylene carbonate

### [Preparation and Application of Resist Composition Coating Solutions]

Components shown in Table 2 below in amounts shown in Table 2 were dissolved in solvents shown in Table 2 to prepare solutions with a solid concentration of 2.7% by mass, and the solutions were filtered using polyethylene filters having a pore size of 0.02 m to obtain resist compositions R-1 to R-14, RX-1, and RX-2.

In Table 2 below, when two or more types of components were used, their types and amounts used were shown with “/” between them. For example, in Example 11, “(A-11)/(A-1)” means that two resins (A), i.e., (A-11) and (A-1), were used, and “5/5” means that 5 g of (A-11) and 5 g of (A-1) were used.

One of the resist compositions obtained was applied to a 6 inch Si (silicon) wafer pre-treated with hexamethyldisilazane (HMDS) using a spin coater Mark 8 manufactured by Tokyo Electron Ltd. and dried on a hot plate at 130° C. for 300 seconds to thereby obtain a resist film having a thickness of 100 nm.

Even when the Si wafer was changed to a chromium substrate, the same results were obtained.

TABLE 2

| | | | | Photoacid | | Acid diffusion | | Acidic compound | | | | | |
|---|---|---|---|---|---|---|---|---|---|---|---|---|---|
| | | Resin (A) | | generator (B) | | control agent | | (F) | | Surfactant | | | |
| | | | Amount | | Amount | | Amount | | Amount | | Amount | Solvent | |
| | Resist | | used | | used | | used | | used | | used | | Mass |
| | composition | Type | (g) | Type | (g) | Type | (g) | Type | (g) | Type | (g) | Type | ratio |
| Example 1 | R-1 | (A-1) | 10 | (B-1) | 1.00 | (D-1) | 0.2 | (F-1) | 0.1 | W-1 | 0.003 | SL-1/SL-5 | 60/40 |
| Example 2 | R-2 | (A-2) | 10 | (B-2) | 1.00 | (D-2) | 0.2 | (F-2) | 0.1 | W-3 | 0.003 | SL-1/SL-2 | 60/40 |
| Example 3 | R-3 | (A-3) | 11 | — | — | (D-3) | 0.2 | (F-3) | 0.1 | W-6 | 0.003 | SL-1/SL-8 | 70/30 |
| Example 4 | R-4 | (A-4) | 10 | (B-3) | 1.00 | (D-4) | 0.2 | (F-4) | 0.1 | W-6 | 0.003 | SL-1/SL-5 | 70/30 |
| Example 5 | R-5 | (A-5) | 11 | — | — | (D-3) | 0.2 | (F-5) | 0.1 | W-1 | 0.003 | SL-1/SL-7 | 70/30 |
| Example 6 | R-6 | (A-6) | 10 | (B-1) | 1.00 | (D-4) | 0.2 | (F-6) | 0.1 | W-5 | 0.003 | SL-1/SL-5 | 90/10 |
| Example 7 | R-7 | (A-7) | 10 | (B-4) | 1.00 | (D-1) | 0.2 | (F-7) | 0.1 | W-4 | 0.003 | SL-1/SL-5 | 60/40 |
| Example 8 | R-8 | (A-8) | 11 | (B-3)/ | 0.5/0.5 | (D-3) | 0.2 | (F-8) | 0.1 | W-3 | 0.003 | SL-1/SL-5 | 80/20 |
| | | | | (B-1) | | | | | | | | | |
| Example 9 | R-9 | (A-9) | 10 | (B-4) | 1.00 | (D-4) | 0.2 | (F-9) | 0.1 | W-4 | 0.003 | SL-1/SL-7 | 60/40 |
| Example 10 | R-10 | (A-10) | 10 | (B-6) | 1.00 | (D-1) | 0.2 | (F-10) | 0.1 | W-4 | 0.003 | SL-1/SL-5 | 60/40 |
| Example 11 | R-11 | (A-11)/ | 5/5 | (B-2) | 1.00 | (D-2) | 0.2 | (F-11) | 0.1 | W-5 | 0.003 | SL-1/SL-4 | 80/20 |
| | | (A-1) | | | | | | | | | | | |
| Example 12 | R-12 | (A-12) | 10 | (B-3) | 1.00 | (D-2) | 0.2 | (F-12) | 0.1 | W-2 | 0.003 | SL-1/SL-7 | 60/40 |
| Example 13 | R-13 | (A-13) | 10 | (B-5) | 1.00 | (D-1) | 0.2 | (F-13) | 0.1 | W-3 | 0.003 | SL-1/SL-3 | 60/40 |
| Example 14 | R-14 | (A-1) | 10 | (B-1) | 1.00 | (D-2) | 0.2 | (F-11)/ | 0.05/0.05 | W-1 | 0.003 | SL-1/SL-6 | 50/50 |
| | | | | | | | | (F-13) | | | | | |
| Comparative | RX-1 | (AX-1) | 10 | (B-1) | 1.00 | (D-1) | 0.2 | (F-1) | 0.1 | W-1 | 0.003 | SL-1/SL-5 | 60/40 |
| Example 1 | | | | | | | | | | | | | |
| Comparative | RX-2 | (A-1) | 10 | (B-1) | 1.00 | (D-1) | 0.2 | (FX-1) | 0.1 | W-1 | 0.003 | SL-1/SL-5 | 60/40 |
| Example 2 | | | | | | | | | | | | | |

### [EB Exposure]

Each of the wafers with the resist films applied thereto was subjected to pattern irradiation using an electron beam drawing device (F7000S manufactured by Advantest Corporation, acceleration voltage: 50 keV). In this case, the pattern was drawn such that a line-and-space of 1:1 was formed. After the electron beam drawing, the wafer was heated on a hot plate at 100° C. for 60 seconds, immersed in a 2.38% by mass aqueous tetramethylammonium hydroxide (TMAH) solution for 60 seconds, rinsed with water, and dried. Then the wafer was rotated at a rotation speed of 4000 rpm for 30 seconds and then baked at 95° C. for 60 seconds to dry the wafer.

### [Sensitivity]

A cross-sectional shape of each of the patterns obtained was observed under a scanning electron microscope (S-938011 manufactured by Hitachi, Ltd.). The exposure amount when the 1:1 line-and-space resist pattern with a line width of 50 nm was resolved was used as sensitivity (E₀). The smaller this value, the higher the sensitivity.

### [Resolution]

A cross-sectional shape of each of the patterns obtained was observed under a scanning electron microscope (S-938011 manufactured by Hitachi, Ltd.). The limiting resolving power (the minimum line width at which lines and spaces (line:space 1:1) can be separated and resolved) at the exposure amount when the 1:1 line-and-space resist pattern with a line width of 50 nm was resolved was used as resolution (nm). The smaller this value, the higher the resolution.

### [Bridge Margin]

A line pattern having a line width of 25 nm was exposed to light while the irradiation dose was reduced from E₀ described above. The space width (nm) at which the occurrence of a bridge was found in a space portion between lines of the pattern was used as the indicator of a “bridge margin.” The smaller the above value, the better the performance.

The evaluation results are shown in the “EB evaluation” column in Table 3 below. [Exposure to extreme ultraviolet rays (EUV)]

### [Sensitivity]

Each of the resist films obtained was irradiated with EUV (wavelength: 13 nm) using an EUV exposure device (Micro Exposure Tool manufactured by Exitech, NA (numerical aperture): 0.3, Quadrupole, outer sigma: 0.68, inner sigma: 0.36) to form a pattern. Specifically, while the exposure amount was changed in the range of 0 to 20.0 mJ/cm² in steps of 0.1 mJ/cm², the resist film was exposed to light through a reflective-type mask having a 1:1 line-and-space pattern with a line width of 100 nm and then baked at 110° C. for 90 seconds. Then the resist film was developed using a 2.38% by mass aqueous tetramethylammonium hydroxide (TMAH) solution.

The exposure amount at which the mask pattern having a 1:1 line-and-space pattern (line/space=1/1) with a line width of 100 nm was reproduced was used as sensitivity. The smaller this value, the higher the sensitivity.

### [Resolution]

The limiting resolving power (the minimum line width at which lines and spaces (line:space 1:1) can be separated and resolved) at the exposure amount when the above sensitivity was obtained was used as resolution (nm). The smaller this value, the higher the resolution.

### [Bridge Margin]

A line pattern having a line width of 25 nm was exposed to light while the irradiation dose was reduced from the above-determined sensitively. The space width (nm) at which the occurrence of a bridge was found in a space portion between lines of the pattern was used as the indicator of a “bridge margin.” The smaller the above value, the better the performance.

The evaluation results are shown in the “EUV evaluation” column in Table 3 below.

TABLE 3

| | | EB evaluation | | | EUV evaluation | | |
|---|---|---|---|---|---|---|---|
| | | | | Bridge | | | Bridge |
| | Resist | Sensitivity | Resolution | margin | Sensitivity | Resolution | margin |
| | composition | (μC/cm²) | (nm) | (nm) | (mJ/cm²) | (nm) | (nm) |
| Example 1 | R-1 | 28 | 18 | 17 | 14.5 | 18 | 17 |
| Example 2 | R-2 | 35 | 19 | 18 | 16.1 | 19 | 18 |
| Example 3 | R-3 | 27 | 22 | 20 | 14.1 | 22 | 20 |
| Example 4 | R-4 | 29 | 22 | 21 | 14.8 | 22 | 22 |
| Example 5 | R-5 | 31 | 21 | 20 | 15.3 | 21 | 20 |
| Example 6 | R-6 | 28 | 20 | 20 | 14.6 | 20 | 20 |
| Example 7 | R-7 | 35 | 23 | 23 | 16.1 | 22 | 22 |
| Example 8 | R-8 | 36 | 23 | 23 | 16.1 | 23 | 23 |
| Example 9 | R-9 | 35 | 18 | 19 | 16.2 | 18 | 19 |
| Example 10 | R-10 | 31 | 19 | 20 | 15.5 | 18 | 20 |
| Example 11 | R-11 | 31 | 19 | 20 | 15.6 | 19 | 19 |
| Example 12 | R-12 | 30 | 23 | 22 | 15.4 | 23 | 22 |
| Example 13 | R-13 | 28 | 22 | 23 | 14.7 | 23 | 23 |
| Example 14 | R-14 | 29 | 21 | 20 | 15.1 | 20 | 21 |
| Comparative | RX-1 | 33 | 25 | 24 | 14.9 | 26 | 25 |
| Example 1 | | | | | | | |
| Comparative | RX-2 | 38 | 28 | 24 | 18.9 | 30 | 24 |
| Example 2 | | | | | | | |

As can be seen from the results shown in Table 3, the resist compositions in Examples 1 to 14 can provide good resolution and good bridge margin.

In the resist composition in Comparative Example 1, since the resin (AX-1) having none of the repeating unit represented by general formula (3), the repeating unit represented by general formula (6), and the repeating unit represented by general formula (7) was used, the resolution and bridge margin were worse than those in the resist compositions in the Examples.

In the resist composition in Comparative Example 2, since the compound (FX-1) having no iodine atom was used, the resolution and bridge margin were worse than those in the resist compositions in the Examples.

What is claimed is:

1. An actinic ray-sensitive or radiation-sensitive resin composition comprising: a resin (A) whose polarity increases by the action of an acid; and an acidic compound (F) having an iodine atom,
wherein the resin (A) is a different compound from the acidic compound (F),
wherein the acidic compound (F) is a nonionic compound, and
wherein the resin (A) has at least one selected from the group consisting of a repeating unit represented by general formula (3) below, a repeating unit represented by general formula (6) below, and a repeating unit represented by general formula (7) below: 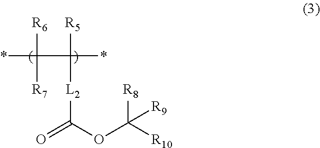
wherein, in the general formula (3),
R₅ to R₇ each independently represent a hydrogen atom, an alkyl group, a cycloalkyl group, a halogen atom, a cyano group, or an alkoxycarbonyl group;
L₂ represents a divalent linking group;
R₈ to R₁₀ each independently represent an alkyl group, a cycloalkyl group, an aryl group, an aralkyl group, or an alkenyl group; and two selected from the group consisting of R₈ to R₁₀ may be bonded together to form a ring; 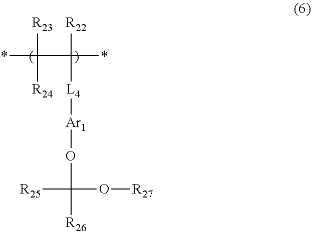
wherein, in the general formula (6),
R₂₂ to R₂₄ each independently represent a hydrogen atom, an alkyl group, a cycloalkyl group, a halogen atom, a cyano group, or an alkoxycarbonyl group;
L₄ represents a single bond or a divalent linking group;
Ar₁ represents an aromatic group;
R₂₅ to R₂₇ each independently represent a hydrogen atom, an alkyl group, a cycloalkyl group, an aryl group, an aralkyl group, or an alkenyl group;
R₂₆ and R₂₇ may be bonded together to form a ring; and
R₂₄ or R₂₅ may be bonded to Ar₁; and 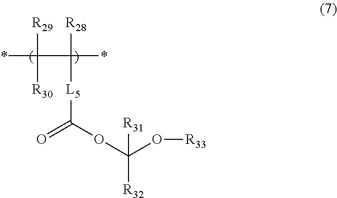
wherein, in the general formula (7),
R₂₈ to R₃₀ each independently represent a hydrogen atom, an alkyl group, a cycloalkyl group, a halogen atom, a cyano group, or an alkoxycarbonyl group;
L₅ represents a single bond or a divalent linking group;
R₃₁ and R₃₂ each independently represent a hydrogen atom, an alkyl group, a cycloalkyl group, an aryl group, an aralkyl group, or an alkenyl group;
R₃₃ represents an alkyl group, a cycloalkyl group, an aryl group, an aralkyl group, or an alkenyl group; and
R₃₂ and R₃₃ may be bonded together to form a ring.

2. The actinic ray-sensitive or radiation-sensitive resin composition according to claim 1, wherein the acidic compound (F) is a compound having an aromatic group substituted with an iodine atom.

3. The actinic ray-sensitive or radiation-sensitive resin composition according to claim 1, wherein the acidic compound (F) is a compound represented by the following general formula (FA1): 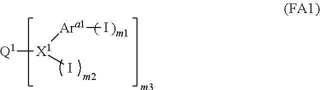
wherein, in the general formula (FA1),
Ar^{a1} represents an aromatic group;
X¹ represents a single bond or a linking group;
Q¹ represents an acidic group;
Q¹ and Ar^{a1} may be bonded together to form a ring;
m1 and m2 each independently represent an integer of 0 to 5, provided that m1+m2 is 1 to 6;
when X¹ represents a single bond, m2 represents 0;
m3 represents 1 or 2; and
when m3 represents 2, two Ar^{a1}'s may be the same or different, and two X¹'s may be the same or different.

4. The actinic ray-sensitive or radiation-sensitive resin composition according to claim 1, wherein the acidic compound (F) is a compound represented by the following general formula (FA2): 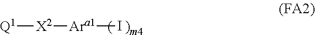
wherein, in the general formula (FA2),
Ar^{a1} represents an aromatic group;
X² represents a single bond or a divalent linking group;
Q¹ represents an acidic group; and
m4 represents an integer of 1 to 5.

5. The actinic ray-sensitive or radiation-sensitive resin composition according to claim 1, wherein the acidic compound (F) is a compound represented by the following general formula (FA3): 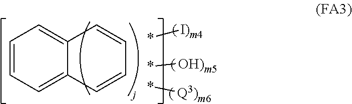
wherein, in the general formula (FA3),
j represents 0 or 1;
Q³ represents a substituent;
m4 represents an integer of 1 to 5;
m5 represents an integer of 1 or more and (6+2j−m4) or less;
m6 represents an integer of 0 or more and (6+2j−m4−m5) or less; and
each * represents a direct bond bonded to an aromatic hydrocarbon shown in the general formula (FA3).

6. The actinic ray-sensitive or radiation-sensitive resin composition according to claim 1, wherein the acidic compound (F) is a compound represented by the following general formula (FA4): 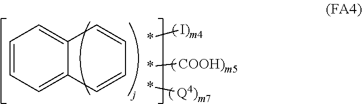
wherein, in the general formula (FA4),
j represents 0 or 1;
Q⁴ represents a substituent;
m4 represents an integer of 1 to 5;
m5 represents an integer of 1 or more and (6+2j−m4) or less;
m7 represents an integer of 0 or more and (6+2j−m4−m5) or less; and
each * represents a direct bond bonded to an aromatic hydrocarbon shown in general formula (FA4).

7. The actinic ray-sensitive or radiation-sensitive resin composition according to claim 1, wherein the acidic compound (F) is a compound represented by the following general formula (FA5): 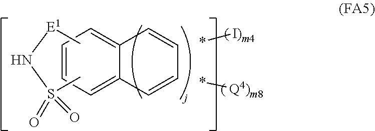
wherein, in the general formula (FA5),
j represents 0 or 1;
Q⁴ represents a substituent;
E¹ represents a single bond or a divalent linking group;
m4 represents an integer of 1 to 5;
m8 represents an integer of 0 or more and (4+2j−m4) or less; and
each * represents a direct bond bonded to an aromatic hydrocarbon shown in the general formula (FA5).

8. The actinic ray-sensitive or radiation-sensitive resin composition according to claim 1, wherein the acidic compound (F) has a pKa of 6 or less.

9. The actinic ray-sensitive or radiation-sensitive resin composition according to claim 1, wherein the acidic compound (F) has a molecular weight of 1000 or less.

10. The actinic ray-sensitive or radiation-sensitive resin composition according to claim 1, wherein the resin (A) has a repeating unit represented by the following general formula (A2): 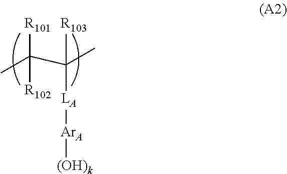
wherein, in the general formula (A2),
R₁₀₁, R₁₀₂, and R₁₀₃ each independently represent a hydrogen atom, an alkyl group, a cycloalkyl group, a halogen atom, a cyano group, or an alkoxycarbonyl group;
L_{A} represents a single bond or a divalent linking group;
Ar_{A} represents an aromatic group;
k represents an integer of 1 to 5;
R₁₀₂ and Ar_{A} may be bonded together; and
when R₁₀₂ and Ar_{A} are bonded together, R₁₀₂ represents a single bond or an alkylene group.

11. The actinic ray-sensitive or radiation-sensitive resin composition according to claim 1, further comprising a compound that generates an acid upon irradiation with actinic rays or radiation.

12. An actinic ray-sensitive or radiation-sensitive film formed from the actinic ray-sensitive or radiation-sensitive resin composition according to claim 1.

13. A pattern forming method comprising:
forming a resist film from the actinic ray-sensitive or radiation-sensitive resin composition according to claim 1;
exposing the resist film to light; and
developing the exposed resist film by a developer.

14. A method for manufacturing an electronic device, the method comprising the pattern forming method according to claim 13.
